(19) United States
(12) Patent Application Publication
(10) Pub. No.: US 2023/0225434 A1
(43) Pub. Date: Jul. 20, 2023

(54) Compression Vest Systems Having Bladders

(71) Applicant: Anthony Thanh Ngo, Cypress, TX (US)

(72) Inventor: Anthony Thanh Ngo, Cypress, TX (US)

(73) Assignee: ACE PT Products and Equipment,INC, Cypress, TX (US)

(21) Appl. No.: 17/575,634

(22) Filed: Jan. 14, 2022

(51) Int. Cl. A41D 1/04; A41D 13/00; A41D 13/12; A61G 7/10

(52) U.S. Cl. CPC A41D 1/04; A41D 13/0007; A41D 13/1245; A61G 7/1038

(57) ABSTRACT

A vest includes a first panel including a first flap, a second panel including a second flap, a back panel extending from the first panel to the second panel, and a plurality of grips. The back panel defines a first and second arm accesses with first and second panels respectively, and portions of the first, second, and back panels disposed below first and second arm accesses define a body of the vest. The first flap is configured to overlap the second flap. The first flap includes a first front attachment segment configured to attach to a second front attachment segment of the second flap in a closed state of the vest. At least one embodiment or aspect may comprise at least one of the following features: at least one zipper expansion and retraction mechanism; and a leg attachment device for detachably securing a compression vest system to a user's leg.

### CROSS-REFERENCE TO RELATED APPLICATIONS

The present application claims the priority benefit of both U.S. Provisional Patent Application Ser. No. 63/136,767, filed on Jan. 13, 2021, and also U.S. Provisional Patent Application Ser. No. 63/136,774, filed on Jan. 13, 2021, which are incorporated herein by reference in their entireties.

### TECHNICAL FIELD

The present disclosure relates to the field of medical and non-medical devices and/or systems that provide chest protection, especially for sports system. More specifically, the present disclosure relates to a compression chest protection system including a chest protection device and a belt. The compression chest protection system is related to a compression vest system disclosed herein by including some or several of the features of the compression vest system disclosed herein. According to certain aspects of the present disclosure, the compression vest system includes a vest and a belt that may be worn by a patient and exert forces on the patient that stabilize the vest in a position relative to the patient as grips attached to the vest and belt are used by a caregiver to aid or ensure the safety of a movement of the patient. Further, the belt may include stays that support and stabilize the patient's spine as the belt compresses a torso of the patient. In addition, at least one embodiment or aspect of a compression vest system and/or a compression chest protection system may comprise at least one of the following four additional features, along with some, most or all of the features of the embodiment(s) or aspect(s) of the present invention that do not include these four additional features: (1) at least one zipper expansion and retraction mechanism, such as, but not limited to, on the right side and/or on and left side of the vest and/or on the right side and/or on and left side of the vest, for allowing users of different body shapes and/or sizes to better fit inside the compression vest system during use; (2) Velcro, hook and loop system, and/or any known securing device, mechanism, or system known to one of ordinary skill in the art on the front and rear of the belt and on the belly button area of the vest to increase stabilization, decrease slippage of the belt from the vest, and increase security of the belt to the vest (especially during transfer or use); (3) at least one swivel hook, ring, attachment ring, and/or any known securing device, mechanism, or system known to one of ordinary skill in the art that are dependent on the type(s) of an individual's needs, sports, therapy, rehabilitation, and settings (as a non-limiting example, this third additional feature is positioned on the vest and belt (five: two on hip of each side of the belt and one on the back side of the belt) to the attachment point); and (4) a leg attachment device for detachably securing a compression vest system to a leg of a user for better stabilization of the compression vest system to the user during transfer or use. Preferably, an embodiment or aspect of a compression vest system comprises most or all of these four additional features.

### BACKGROUND

Situations often arise that require a caregiver to aid a movement of a patient by lifting, guiding, catching, and/or supporting a weight of the patient. The caregiver may be a physical therapist, medical professional, first responder, family member, etc., and the patient may be a person that is injured, is rehabilitating, is elderly, or has limited or no mobility due to a previous injury or terminal illness such as ALS or cerebral palsy. The act of aiding the patient's movement, also know as transferring the patient, involves inherent risks to both the caregiver and the patient.

The caregiver may attempt to handle the patient via a body part of, or clothing worn by, the patient. However, either of these methods can readily lead to the caregiver losing grasp of the patient. In attempting to maintain an original grasp, the caregiver may strain his or her muscles to the point of injury. Additionally, with respect to grasping clothing worn by the patient, it is not uncommon, and in fact should be expected, for the clothing, specifically a location where the clothing is being grasped, to shift positions relative to the patient. The new location of grasping relative to the patient could place either of the caregiver and the patient in an awkward position, or pressure being applied to a sensitive area of a respective person. Generally, this could cause either or both of the caregiver and the patient to experience discomfort, and/or shift a supported load in such a way that muscles or bones of the caregiver or the patient that support the load are loaded or strained to the point of failure and cause injury.

Often times patients are obese or generally of greater size than caregivers such as nurses or physical therapists. Further, some patients may have limited mobility in their arms, hips, and/or legs. Either of these conditions (weight/size disparity or patient mobility) alone, but especially in combination, may cause the patient and/or the caregiver attempting to handle the patient to be in an awkward position and/or support a load that the patient or caregiver is not strong enough to support. Further, trying to re-grasp or catch a patient that is falling is difficult where only a part of the patient's body or a loose piece of clothing is available. These and the other risks discussed herein generally place the caregiver at risk for losing a grasp of and dropping the patient. Just as if the patient fell while doing an activity independent of any aid, dropping the patient may cause the patient to sustain significant injuries. Likewise, in trying to avoid dropping the patient, the caregiver may strain his or her muscles or load his or her bones to the point of injury.

Further, it is often the case that the patient has difficulty maintaining an upright position due to their condition which includes a spine that is functionally inhibited, has a structural irregularity, or is poorly or otherwise insufficiently supported by weak muscles. The patient's spine may persistently exhibit an unnatural curvature, which may only be exacerbated during independent or guided movement by the patient. This can result in further injury to the patient during transfers, as it is difficult for the caregiver to maintain the patient in a safe position. It is not uncommon for caregivers recognizing these conditions to attempt to correct the patient's posture before a transfer, or make impromptu attempts to support and correct the patient's posture during transfers. Such attempts during transfers may involve quickly grasping other parts of the patient's body or other pieces of clothing than originally planned. This can lead to the patient and/or caregiver moving into awkward positions and undue stress being place on the body of the caregiver and/or patient. Accordingly, a lack of spinal stability in the patient can result in further injuries to the caregiver and the patient when moving or guiding the movement of the patient.

These and other issues are addressed by a vest compression system and method of transferring a patient using the vest compression system of the present disclosure.

### SUMMARY

According to certain aspects of the present disclosure, a vest includes a first panel including a first flap, a second panel including a second flap, a back panel extending perpendicular to a longitudinal axis of the vest from the first panel to the second panel, and a plurality of grips attached to the first panel, the second panel, and the back panel. According to other aspects of the present disclosure the back panel defines a first arm access with the first panel and a second arm access with the second panel, and portions of the first panel, second panel, and back panel disposed along a longitudinal axis of the vest below the first arm access and the second arm access define a body of the vest. According to other aspects of the present disclosure, the first flap is configured to overlap the second flap such that the first flap and the second flap define a closure of the vest and the first panel and the second panel define a collar of the vest, the first flap includes a first front attachment segment and the second flap includes a second front attachment segment configured to attach to the first front attachment segment in a closed state of the vest, and an area of an attachment configuration between the first front attachment segment and the second front attachment segment in the closed state of the vest is equal to at least ⅔ of an area having a length equal to a length of the body and a width equal to ¼ of a maximum width of the body

According to other certain aspects of the present disclosure, a compression vest system includes a vest having a back panel including a back attachment segment provided on an exterior surface of the back panel, a first panel extending from the back panel, a second panel extending from the back panel, and a plurality of grips attached to at least the first panel and the second panel. According to other aspects of the present disclosure, the compression vest system further includes a belt having a pair of flanks, a first belt attachment segment positioned on an interior surface of an end of one of the pair of flanks, a second belt attachment segment positioned on interior surface of the belt between the pair of flanks and configured to attach to the back attachment segment, and a pair of elastic tension bands attached to an exterior surface of the belt between the pair of flanks. According to other aspects of the present disclosure, the first panel and the second panel define a closure of the vest that extends along a longitudinal axis of the vest and has a width equal to at least ¼ of a maximum width of the vest, and the closure is configured to exert first compressive forces on an individual wearing the vest. According to other aspects of the present disclosure, the pair of flanks are configured to wrap around the vest such that one flank overlaps an other flank and the pair of flanks exert second compressive forces on the individual, and each of the pair of elastic tension bands is configured to be attached to a third belt attachment segment positioned on an exterior surface of a respective one of the pair of flank in a state of tension and increase the second compressive forces.

According to other certain aspects of the present disclosure, a method of transferring a patient includes providing a vest including a first panel, a second panel, and a back panel extending from the first panel and the second panel, positioning the vest on the patient such that the vest exerts first compressive forces on the patient, wrapping a pair of flanks of the belt around the vest such that the belt exerts second compressive forces on the patient, attaching an end of each of a pair of elastic tension bands to an attachment segments positioned on respective one of the pair of the flanks such that each elastic tension band is attached in a state of tension and increases a magnitude of the second compressive forces, and grasping one or more of a plurality of grips attached to the vest and performing at least one of guiding and supporting a movement of the patient.

According to other certain aspects of the present disclosure, at least one embodiment or aspect of a compression vest system and/or a compression chest protection system may comprise at least one of the following four additional features, along with some, most or all of the features of the embodiment(s) or aspect(s) of the present invention that do not include these four additional features: (1) at least one zipper expansion and retraction mechanism, such as, but not limited to, on the right side and/or on and left side of the vest and/or on the right side and/or on and left side of the vest, for allowing users of different body shapes and/or sizes to better fit inside the compression vest system during use; (2) Velcro, hook and loop system, and/or any known securing device, mechanism, or system known to one of ordinary skill in the art on the front and rear of the belt and on the belly button area of the vest to increase stabilization, decrease slippage of the belt from the vest, and increase security of the belt to the vest (especially during transfer or use); (3) at least one swivel hook, ring, attachment ring, and/or any known securing device, mechanism, or system known to one of ordinary skill in the art that are dependent on the type(s) of an individual's needs, sports, therapy, rehabilitation, and settings (as a non-limiting example, this third additional feature is positioned on the vest and belt (five: two on hip of each side of the belt and one on the back side of the belt) to the attachment point); and (4) a leg attachment device for detachably securing a compression vest system to a leg of a user for better stabilization of the compression vest system to the user during transfer or use. Preferably, an embodiment or aspect of a compression vest system comprises most or all of these four additional features. According to other certain aspects of the present disclosure, a method of transferring a patient includes providing a compression vest system that includes at least one of the four additional features.

### BRIEF DESCRIPTION OF THE DRAWINGS

FIG. 1A illustrates a caregiver aiding a movement of a patient.

FIG. 1B illustrates an implementation of a compression vest system, according to an aspect of the present disclosure.

FIGS. 2A and 2B illustrate front and rear perspective views of a vest of a compression vest system, according to an aspect of the present disclosure.

FIG. 3 illustrates a front elevation view of a vest in a closed state, according to an aspect of the present disclosure.

FIG. 4 illustrates a front elevation view of a vest in a first open state, according to an aspect of the present disclosure.

FIG. 5 is an enlarged view of a portion of FIG. 4, identified as Detail 5.

FIG. 6 is an enlarged view of a portion of FIG. 4, identified as Detail 6.

FIG. 7 illustrates an elevation view of a front of a vest in a second open state, according to an aspect of the present disclosure.

FIG. 8 is an enlarged view of a portion of FIG. 3, identified as Detail 8.

FIG. 9 is an enlarged view of a portion of FIG. 4, identified as Detail 9.

FIG. 10 is an enlarged view of a portion of FIG. 4, identified as Detail 10.

FIG. 11 illustrates an elevation view of a back region of a vest, according to an aspect of the present disclosure.

FIG. 12A illustrates an elevation view of an interior surface of a belt of a compression vest system in a passive state, according to an aspect of the present disclosure.

FIG. 12B illustrates an elevation view of an exterior surface of a belt of a compression system in a passive state, according to an aspect of the present disclosure.

FIG. 12C illustrates an elevation view of an exterior surface of a belt of a compression system in an active state, according to an aspect of the present disclosure.

FIGS. 13A and 13B illustrate front and rear perspective views of a compression vest system, according to an aspect of the present disclosure.

FIG. 13C illustrates a front perspective view of a compression vest system and sagittal, coronal, and transverse body planes.

FIG. 14 illustrates a side elevation view of a vest, according to an aspect of the present disclosure.

FIG. 15A illustrates an elevation view of a side of a vest in a closed state, according to an aspect of the present disclosure.

FIG. 15B illustrates an elevation view of a side of a vest in an open state, according to an aspect of the present disclosure.

FIG. 16 illustrates a side elevation view of a vest in a closed state, according to an aspect of the present disclosure.

FIG. 17 illustrates a compression vest according to an aspect of the present disclosure.

FIG. 18 illustrates a compression belt according to an aspect of the present disclosure.

FIGS. 19A and 19B overhead views of a cloak, according to aspect of the present disclosure.

FIG. 20 illustrates an implementation of an integrated compression vest system, according to aspect of the present disclosure.

FIGS. 21A and 21B illustrate front and rear perspective views of a partial implementation of a compression vest system, according to an aspect of the present disclosure.

FIGS. 22A and 22B illustrate front and rear perspective views of a full implementation of a compression vest system, according to an aspect of the present disclosure.

FIGS. 23A and 23B illustrate elevation views of an exterior and an interior of a belt in a compact state.

FIGS. 23C and 23D illustrate elevation views of an exterior and an interior of a belt in an expanded state.

FIG. 24 illustrates an elevation view of a belt including removable stays, according to an aspect of the present disclosure.

FIG. 25 illustrates a front isometric view of a stay.

FIG. 26 illustrates an enlarged view of a portion of FIG. 4 including a zipper.

FIG. 27 illustrates an enlarged view of a portion of FIG. 4 including a hoop and loop access.

FIG. 28 illustrates an elevation view of a belt including narrow sized stays, according to an aspect of the present disclosure.

FIG. 29 illustrates an elevation view of a belt including intermediate sized stays, according to an aspect of the present disclosure.

FIG. 30 illustrates an elevation view of a belt including short stays, according to an aspect of the present disclosure.

FIG. 31 illustrates an elevation view of a belt including wide stays and an anterior segmented pouch, according to an aspect of the present disclosure.

FIG. 32 illustrates an elevation view of a belt, according to an aspect of the present disclosure.

FIGS. 33A and 33B illustrate a front and rear elevation views of a modular segmented pouch, according to an aspect of the present disclosure.

FIG. 34 illustrates an elevation view of a belt with a modular pouch attached, according to an aspect of the present disclosure.

FIG. 35 illustrates an elevation view of a belt with a modular pouch attached, according to an aspect of the present disclosure.

FIG. 36 illustrates an elevation view of a belt including anterior pocket, according to an aspect of the present disclosure.

FIG. 37 illustrates a modular pouch, according to an aspect of the present disclosure.

FIGS. 38A and 38B illustrate front and rear perspective views of a full implementation of a compression vest system including an extra-wide stay and a modular segmented pouch.

FIG. 39 illustrates an elevation view of a belt including an extended central attachment segment, according to an aspect of the present disclosure.

FIG. 40 illustrates a modular segmented pouch, according to an aspect of the present disclosure.

FIGS. 41A and 41B illustrate front and rear perspective views of a full implementation of a compression vest system including a belt with multiple modular segmented pouches, according to an aspect of the present disclosure.

FIG. 42 illustrates a front view of a compression vest system according to an aspect of the present disclosure, wherein the compression vest system is in a closed position.

FIG. 43 illustrates a front, left side, perspective view of the compression vest system of FIG. 42, wherein the zipper expansion and retraction mechanism of the vest is in a closed position while the zipper expansion and retraction mechanism of the belt is in an open position.

FIG. 44 illustrates a front, left side, perspective view of the compression vest system of FIG. 42, wherein the vest is in a partially open position while the zipper expansion and retraction mechanism of the vest is in a closed position.

FIG. 45 illustrates a right side view of the compression vest system of FIG. 42, wherein the belt is in an open position while the zipper expansion and retraction mechanism of the vest is in an open position.

FIG. 46 illustrates a front view of the compression vest system of FIG. 42, wherein the compression vest system is in an open position.

FIG. 47 illustrates a front view of the vest of the compression vest system of FIG. 42, wherein the vest is in the open position.

FIG. 48 illustrates a front, right side view of the vest of the compression vest system of FIG. 42, wherein the right side of the vest is in the open position.

FIG. 49 illustrates a front, left side view of the vest of the compression vest system of FIG. 42, wherein the left side of the vest is in the open position.

FIG. 50 illustrates a front view of a compression vest system according to an aspect of the present disclosure, wherein the compression vest system is in a closed position.

FIG. 51 illustrates a front, right side, perspective view of the compression vest system of FIG. 50, wherein the zipper expansion and retraction mechanism of the vest is in an open position and the zipper expansion and retraction mechanism of the belt is in an open position.

FIG. 52 illustrates a front, left side, perspective view of the compression vest system of FIG. 50, wherein the zipper expansion and retraction mechanism of the vest is in a closed position and the zipper expansion and retraction mechanism of the belt is in a closed position.

FIG. 53 illustrates a front, right side, perspective view of the belt of the compression vest system of FIG. 50, wherein the zipper expansion and retraction mechanism of the belt is in an open position.

FIG. 54 illustrates a front, right side, perspective view of the vest of the compression vest system of FIG. 50, wherein the zipper expansion and retraction mechanism of the vest is in an open position.

FIG. 55 illustrates a rear view of the compression vest system of FIG. 50.

FIG. 56 illustrates a rear, right side view of the compression vest system of FIG. 50, wherein the zipper expansion and retraction mechanism of the vest is in an open position and the zipper expansion and retraction mechanism of the belt is in an open position.

FIG. 57 illustrates a rear, left side view of the compression vest system of FIG. 50, wherein the zipper expansion and retraction mechanism of the vest is in a closed position and the zipper expansion and retraction mechanism of the belt is in a closed position.

FIG. 58 illustrates a rear, left side view of the vest of the compression vest system of FIG. 50, wherein the zipper expansion and retraction mechanism of the vest is in a closed position.

FIG. 59 illustrates a front view of a compression binding vest system according to an aspect of the present disclosure, wherein the compression binding vest system is in a closed position.

FIG. 60 illustrates a rear view of the compression binding vest system of FIG. 59, wherein the zipper expansion and retraction mechanism of the left side of the upper belt is in a closed position and the zipper expansion and retraction mechanism of the left side of the lower belt is in an open position.

FIG. 61 illustrates a rear view of the compression binding vest system of FIG. 59, wherein the zipper expansion and retraction mechanism of the left side of the upper belt is in the closed position and the zipper expansion and retraction mechanism of the left side of the lower belt is in the open position.

FIG. 62 illustrates a rear view of the compression binding vest system of FIG. 59, wherein the zipper expansion and retraction mechanism of the right side of the upper belt is in the open position and the zipper expansion and retraction mechanism of the right side of the lower belt is in the open position.

FIG. 63 illustrates a front, right side view of an upper belt of the compression binding vest system of FIG. 59.

FIG. 64 illustrates a front, left side view of an upper belt of the compression binding vest system of FIG. 59.

FIG. 65 illustrates a rear, right side view of the compression binding vest system of FIG. 59, wherein the zipper expansion and retraction mechanism of the right side of the upper belt is in the open position.

FIG. 66 illustrates a rear, left side view of the compression binding vest system of FIG. 59, wherein the zipper expansion and retraction mechanism of the left side of the upper belt is in the closed position.

FIG. 67 illustrates a front, environmental view of a compression binding vest system according to an aspect of the present disclosure, wherein the compression binding vest system is in a closed position.

FIG. 68 illustrates a rear, environmental view of the compression binding vest system of FIG. 67, wherein the compression binding vest system is in the closed position.

FIG. 69 illustrates a left side view of the compression binding vest system of FIG. 67, wherein the compression binding vest system is in the closed position.

FIG. 70 illustrates a front, environmental view of a compression binding vest system according to an aspect of the present disclosure, wherein the compression binding vest system is in a closed position.

FIG. 71 illustrates a rear, environmental view of the compression binding vest system of FIG. 70, wherein the compression binding vest system is in the closed position.

FIG. 72 illustrates a left side view of the compression binding vest system of FIG. 70, wherein the compression binding vest system is in the closed position.

FIG. 73 illustrates a right side view of the compression binding vest system of FIG. 70, wherein the compression binding vest system is in the closed position.

FIG. 74 illustrates a front view of a leg attachment device according to an aspect of the present disclosure.

FIG. 75 illustrates an environmental, rear, right side view of the leg attachment device of FIG. 74.

FIG. 76 illustrates an environmental, left side view of the leg attachment device of FIG. 74.

FIG. 77 illustrates an environmental, rear, left side view of the leg attachment device of FIG. 74.

FIG. 78 illustrates a perspective, front, right side view of a vest and bladder system according to an aspect of the present disclosure.

FIG. 79 illustrates a perspective, front, bottom, right side view of the vest and bladder system of FIG. 78.

FIG. 80 illustrates a perspective, front, right side, upright view of the vest and bladder system of FIG. 78.

FIG. 81 illustrates a perspective, front, bottom, right side view of the vest and bladder system of FIG. 78.

FIG. 82 illustrates a perspective, front, bottom view of the vest and bladder system of FIG. 78.

FIG. 83 illustrates a perspective, front, bottom, right side view of another vest and bladder system according to an aspect of the present disclosure.

FIG. 84 illustrates a perspective, front, left side, upright view of the vest and bladder system of FIG. 83.

FIG. 85 illustrates a perspective, front, right side, upright view of the vest and bladder system of FIG. 83.

FIG. 86 illustrates a perspective, front, bottom view of a further vest and bladder system according to an aspect of the present disclosure.

FIG. 87 illustrates a perspective, rear, bottom view of the vest and bladder system of FIG. 86.

FIG. 88 illustrates a left side view of the vest and bladder system of FIG. 86.

FIG. 89 illustrates a perspective, front view of a bladder system according to an aspect of the present disclosure.

FIG. 90 illustrates a rear view of a cervical and head region of a skeleton.

FIG. 91 illustrates a perspective, front view of another vest and bladder system according to an aspect of the present disclosure.

FIG. 92 illustrates a perspective, front view of the vest and bladder system of FIG. 91.

FIG. 93 illustrates a perspective, front view of a further vest and bladder system according to an aspect of the present disclosure.

FIG. 94 illustrates a perspective, front view of the vest and bladder system of FIG. 93.

FIG. 95 illustrates a front view of an upper section of the vest and bladder system of FIG. 93.

FIG. 96 illustrates a front view of a head and neck bladder device of the vest and bladder system of FIG. 93.

FIG. 97 illustrates a front view of the head and neck bladder device of FIG. 96.

FIG. 98 illustrates a front view of the head and neck bladder device of FIG. 96.

FIG. 99 illustrates a front view of the head and neck bladder device of FIG. 96.

FIG. 100 illustrates a front view of the head and neck bladder device of FIG. 96.

FIG. 101 illustrates a front view of the head and neck bladder device of FIG. 96.

FIG. 102 illustrates a front view of the head and neck bladder device of FIG. 96.

FIG. 103 illustrates a front view of the head and neck bladder device of FIG. 96.

FIG. 104 illustrates an environmental, rear view of the vest and bladder system of FIG. 93.

FIG. 105 illustrates an environmental, perspective, rear, upper right side view of the vest and bladder system of FIG. 93.

FIG. 106 illustrates an environmental, rear view of the vest and bladder system of FIG. 93.

FIG. 107 illustrates an environmental, perspective, rear, right side view of the vest and bladder system of FIG. 93.

FIG. 108 illustrates a perspective, front view of another head and neck bladder device according to an aspect of the present disclosure.

FIG. 109 illustrates a perspective, rear view of the head and neck bladder device of FIG. 108.

FIG. 110 illustrates a perspective, rear, right side view of the head and neck bladder device of FIG. 108.

FIG. 111 illustrates an environmental, perspective, front, left side view of the head and neck bladder device of FIG. 108.

### DETAILED DESCRIPTION

Aspects of the disclosure will now be described in detail with reference to the figures, wherein like reference numbers refer to like elements throughout, unless specified otherwise. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

Aspects of the present disclosure described herein are directed toward a compression vest system that includes a vest and a belt. The vest may include a back panel, a first panel that may extend from the back panel and define a first arm access, and a second panel that may extend from the back panel and define a second arm access opposite to the first arm access. A body of the vest may be defined by those portions of the back, first, and second panels that extend from a lower end of the vest to an axis passing through a lower end of each of the first and second arm access. The first panel may include a first flap that overlaps a second flap of the second panel in a closed state of the vest.

An interior surface of the first flap may include a first front attachment segment that is configured to attach to a second front attachment segment provided on an exterior surface of the second flap, such that the first and second flaps define a closure of the vest. A configuration of portions of the first front attachment segment configured to mirror and attach to portions of the second front attachment segment (or vice versa) in a closed state of the vest may define an attachment configuration of the closure. An area of a fully implemented attachment configuration may be sized relative to an area defined by a length and width of the body such that when the vest is fittingly worn by an individual, the vest: (1) may exert first compressive forces on the body of the individual; and (2) remain stationary relative to a respective position on the individual in response to an external force being applied to the vest.

The belt may include a pair of flanks, a pair of elastic tension bands, and a belt attachment segment configured to attach to a back attachment segment provided on the back panel of the vest. The belt may be attached to the vest and form-fittingly wrapped around the vest being worn by the patient and exert second compressive force on a torso of the patient. The elastic tension bands may be extended in opposite directions beyond normal state lengths and attached to belt attachment segments provided on respective flanks to increase the second compressive forces exerted on the patient.

A plurality of inelastic grips may be attached to exterior surfaces of the back, first, and second panels of the vest, as well as an exterior surface of the belt. The grips may allow a caregiver, regardless of size relative to a patient, to select a combination of grips to maximize a degree of leverage the caregiver is capable of comfortably and safely implementing relative to the patient, to aid a movement of the patient. Further, the grips may be relied on to provide a constant grip and minimize a risk of the caregiver having to re-grip, or dropping the patient. During the use of the grips as discussed, the first and second compressive forces exerted on the patient via implementations of the closure and the belt, ensures the vest remains in a position (stationary position) relative to the patient that, inter alia: (A) allows the caregiver to continue to safely move or guide the patient; and (B) minimize any discomfort to the patient that may result from the vest bunching up or moving to touch various body parts like a neck of the patient.

In addition, aspects of the present disclosure described herein are directed toward a compression vest system that includes a vest, a belt, and at least one stay. The vest may include a back panel, a first panel that may extend from the back panel and define a first arm access, and a second panel that may extend from the back panel and define a second arm access opposite to the first arm access. A body of the vest may be defined by those portions of the back, first, and second panels that extend from a lower end of the vest to an axis passing through a lower end of each of the first and second arm access. The first panel may include a first flap that overlaps a second flap of the second panel in a closed state of the vest.

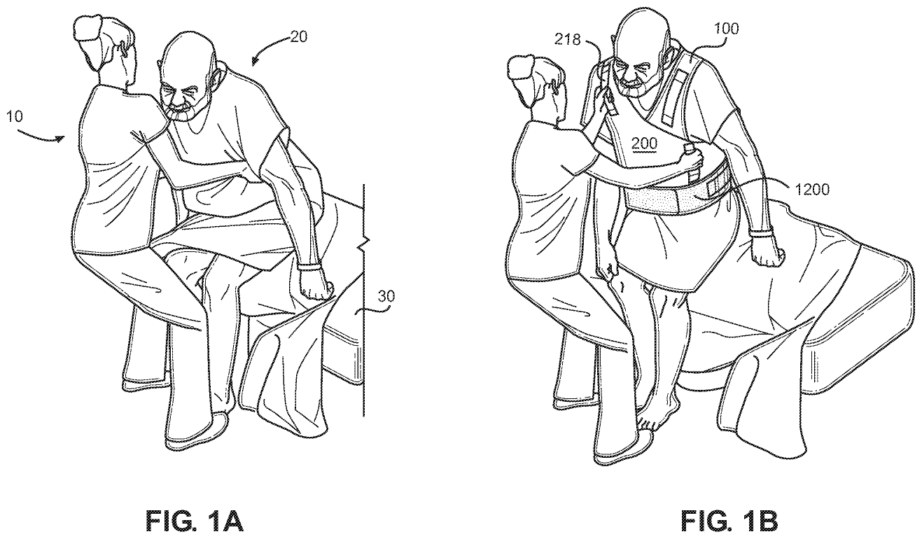

FIG. 1A illustrates a caregiver 10 aiding a movement of a patient 20. As illustrated in FIG. 1A, the caregiver 10 is smaller than the patient 20, and is required to directly support a substantial weight of the patient 20 in order to move the patient 20 from a bed 30. In addition, the caregiver 10 must directly handle (e.g. grab) the patient 20. One of ordinary skill in the art will recognize that as a consequence of the caregiver 10 supporting the load generated by the weight of the patient 20, the caregiver 10 will experience various magnitudes of stress in a variety of muscles and bones, for example in the areas of the back, knees, elbows, and/or wrists. Accordingly, the caregiver 10 may be at risk of sustaining an injury to one or more of these areas.

In addition, handling the patient 20 directly may involve holding on to a body part of the patient 20 or a piece of clothing the patient 20 is wearing. It follows that the caregiver 10 may be at risk of losing their respective grasp of the patient 20 and be forced to re-grip and/or adjust a hand placement. The attendant risk in such a maneuver, which could occur in the situation illustrated in FIG. 1A, is that the new hand placement may cause the caregiver 10 to support the patient 20 in an uncomfortable and unorthodox manner exposing both to injury, or be ineffective and lead to patient 20 being dropped.

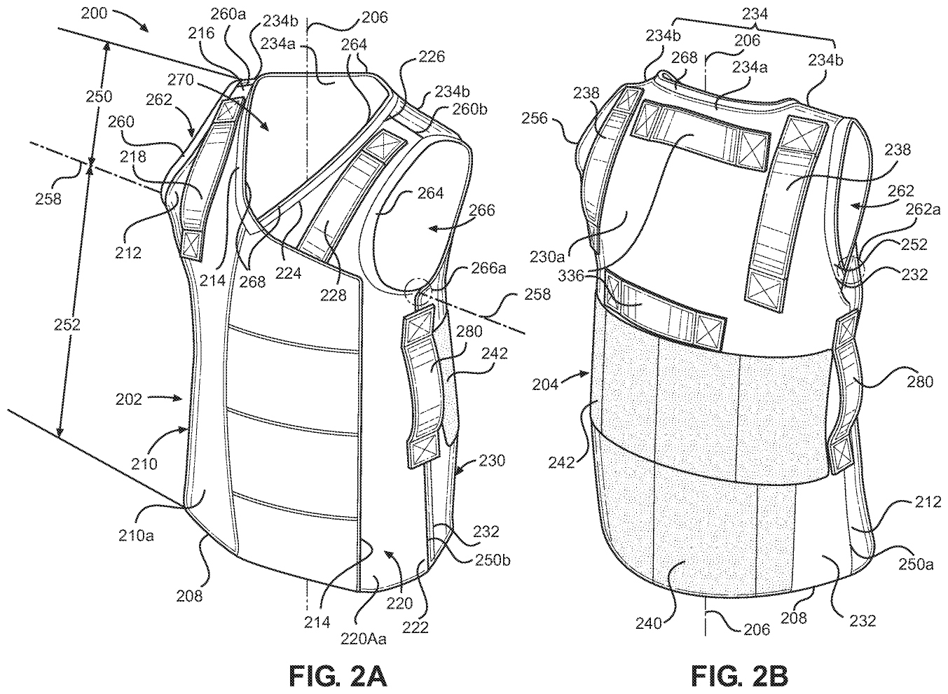

FIG. 1B illustrates the caregiver 10 aiding a movement of the patient 20 utilizing a compression vest system 100, according to an aspect of the present disclosure. The compression vest system 100 includes a vest 200, which is described in more detail with reference to FIGS. 2A-11, and a belt 1200, which is described in more detail with reference to FIGS. 12A-C. In a normal implementation of the compression vest system 100, the compression vest system 100 may be worn by the patient 20 so as to apply radially compressive forces to the body (torso) of the patient 20 as explained in further detail herein. These compressive forces prevent the vest 200 from shifting relative to the patient 20 when external forces are applied to grips such as a first grip 218, a body grip 280, and/or a belt grip 1220 illustrated in FIG. 1B, attached to the vest 200 and/or the belt 1200.

One of ordinary skill in the art will recognize that at least relative to the clothing or body of the patient 20, the compression vest system 100 provides the caregiver 10 with a more rigid and usable device for pulling, lifting, guiding, or generally moving the patient 20. In particular, the vest compression system 100 incorporates grips that are substantially inelastic and rigidly attached to a garment (vest 200) that remains stationary relative to the patient 20. As a result, the caregiver 10, irrespective of a size relative to the patient 20, can select a combination of grips to use that: (A) will maximize a degree of leverage the caregiver 10 is capable of comfortably and safely implementing relative to the patient 20 to aid a movement thereof; and (B) can be relied on to provide a constant grip and minimize a risk of having to re-grip, or dropping the patient 20.

As defined herein a patient may be any individual requiring assistance to move in any manner, and a caregiver may be any individual attempting to aid the patient and need not be a medical professional such as a doctor, nurse, or physical therapist.

FIGS. 2A and 2B illustrate front and rear perspective views of the vest 200 of the compression vest system 100, according to an aspect of the present disclosure. The vest 200 includes a front region 202 and a back region 204. The front region 202 is defined by a first panel 210 and a second panel 220; both extending from a back panel 230 that defines the back region 204. The vest 200 extends along a first longitudinal axis 206 from a lower end 208 thereof, and defines a head 250 and a body 252. Further, the back panel 230 extends perpendicular to the first longitudinal axis 206 from the first panel 210 to the second panel 220. According to an aspect of the present disclosure, the first panel 210 may be attached to the back panel via a first side seam 254a as illustrated in FIG. 2B. The first side seam 254a may be provided between an outer edge 212 of the first panel 210 (“first outer edge 212”), and a corresponding portion of a back outer edge 232 of the back panel 230. Likewise, the second panel 220 may be attached to the back panel 230 via a second side seam 254b as illustrated in FIG. 2A. The second side seam 254b may be provided between an outer edge 222 of the second panel 220 (“second outer edge 222”) and a corresponding portion of the back outer edge 232. The side seams 254a, 254b may extend parallel to the longitudinal axis 206 as illustrated, or angled relative to the longitudinal axis 206. The side seams 254a, 254b may be constructed with stitching or reinforcement stitching.

The first panel 210, the second panel 220, and the back panel 230 may be formed from the same material or different materials. According to an aspect of the present disclosure the first, second, and back panels 230 may be formed from a fabric material such as canvas. According to another aspect of the present disclosure, the first panel 210, the second panel 220, and the back panel 230 may be formed from fire retardant or water proof material. In addition, portions of interior and exterior surfaces of the vest 200 may be lined with cotton or fleece material to provide added comfort to a patient wearing or a caregiver touching the vest 200. Further, portions of the first panel 210, the second panel 220, and the back panel 230 may be formed from elastic material.

As illustrated in FIGS. 2A and 2B, the first panel 210 further includes a first upper edge 216 that is attached via a first upper seam 256a to a corresponding peripheral portion 234b of a back upper edge 234 of the back panel 230. The second panel 220 includes a second upper edge 226 that is attached via a second upper seam 256b to a corresponding peripheral portion 234b of the back upper edge 234.

One of ordinary skill in the art will recognize that the first panel 210 and/or the second panel 220 may be formed as one piece with the back panel 230 in one or more of the areas corresponding to the side seams 254a, 254b and the upper seam 256a, 256b. Thus, according to an aspect of the present disclosure, any combination or all of the seams 254a, 254b, 260a, 256b discussed above may be obviated. However, in any of the configurations discussed herein, a portion of the first outer edge 212 will extend from the first upper edge 216 (or an area corresponding to the first upper edge 216) to a location corresponding to a transition 258 between the head 250 and the body 252 of the vest 200 as identified in FIGS. 2A and 2B. This portion of the first outer edge 212 covered by a first outer lip 260, along with a corresponding portion of the back outer edge 232 also covered by the first outer lip 260, defines a first arm access 262. Further, a portion of the second outer edge 222 will extend from the second upper edge 226 (or an area corresponding to the first upper edge 216) to the transition 258. This portion of the second outer edge 222 covered by a second outer lip 264, along with a corresponding portion of the back outer edge 232 also covered by the second outer lip 264, defines a second arm access 266. It will be understood that the transition 258 coincides with an axis that passes through a lower end 262a of the first arm access 262 and a lower end 266a of the second arm access 266.

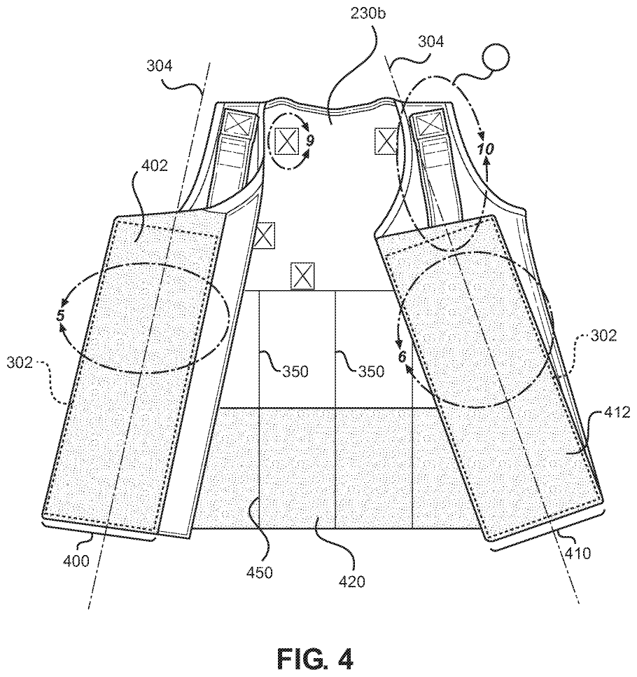

With respect to the first panel 210, an exterior surface 210a of the first panel 210 (“first exterior surface 210a”) is opposite an interior surface of the first panel 210 (“first interior surface 210b”—see FIG. 4), and extends from the first outer edge 212 to a first inner edge 214. The first inner edge 214 extends from the first upper edge 216 to the lower end 208 of the body 252. In a closed state of the vest 200 illustrated in FIG. 2A, the first panel 210 overlaps the second panel 220 and the first inner edge 214 extends across and down the second panel 220. Accordingly, the first inner edge 214 defines a free edge of the front region 202. Further with respect of the first panel 210, a first front grip 218 is attached to the first panel 210 just below the first upper edge 216 and extends over a substantial portion of the head 250 of the vest 200. One of ordinary skill in the art will recognize that the first front grip 218 may be provided in different lengths and cover different proportions of the head 250.

The first and second front grips 218, 228 may be positioned as illustrated in FIGS. 2A and 2B so that a caregiver can easily access a grip as it will be positioned near or on the shoulders of a patient. Further, by positioning the first and second front grips 218, 228 so as to be near to a head of the patient when the vest 200 is being worn, the patient's head will likely be in the caregiver's line of sight if the front grips 218, 228 are used. Accordingly, the caregiver will be able to easily see how his or her manipulation of the front grips 218, 228 to move/transfer the patient affects a movement of the patient's head. As result, the caregiver may quickly adjust the manner in which the front grips 218, 228 are being manipulated to minimize discomfort or the chance that the patient's neck will be subject to abrupt movements of the patient's head.

With respect to the second panel 220, an exterior surface 220a of the second panel 220 (“second exterior surface 220a”) is opposite an interior surface 220b of the second panel 220 (“second interior surface 220b”—see FIG. 7), and extends from the second outer edge 222 to a second inner edge 224. The second inner edge 224 extends from the second upper edge 226 to the lower end 208 of the body 252. In a closed state of the vest 200 illustrated in FIG. 2A, the first panel 210 overlaps the second panel 220, and the first panel 210 covers a substantial portion of the second inner edge 224 extending over the body 252. A second front grip 228 is attached to the second panel 220 just below the second upper edge 226 and extends over a substantial portion of the head 250 of the vest 200. One of ordinary skill in the art will recognize that the second front grip 228 may be provided in different lengths and cover different proportions of the head 250.

As discussed above with respect to the back panel 230, portions of the back outer edge 232 define the first arm access 262 and the second arm access 266. In addition, a middle portion 234a of the back upper edge 234 is covered by an upper lip 268 and defines a collar 270 along with portions of the first inner edge 214 and the second inner edge 224 also covered by the upper lip 268.

The back panel 230 is defined by a back-exterior surface 232a illustrated in FIGS. 2A and 2B, and a back interior surface 232b, a portion of which being visible in FIG. 2A. Further, as shown in FIG. 2B, a set of first back grips 236 and a set of second back grips 238 are attached to the back-exterior surface 232a. The first back grips 236 extend laterally and the second back grips 238 extend vertically, and each set includes two grips. One first back grip 236 is positioned proximate to the back upper edge 234 and the other first back grip 236 is disposed at or below the transition 258 between the head 250 and the body 252. Thus, the second back grips 238 and at least one of the first back grips 236 are attached to the back panel 230 within the head 250. However, one of ordinary skill in the art will recognize that the first back grips 236 and the second back grips 238 may be positioned closer together or farther apart, in other orientations (for example the second back grips 238 may be angled in a V-shape or A-shape), and provided in different numbers per set (e.g. 3 or 4 per set). The back panel 230 also includes a first back attachment segment 240 and second back attachment segment 242 positioned below the transition 258.

As defined herein, an attachment segment includes a layer of interlocking material, such as hook and loop fastener material, that is capable of being fastened (e.g. stitched, glued, molded) to a surface of the vest 200 (i.e. a surface of the first, second, or back panel 210, 220, 230) or the belt 1200. A layer of interlocking material enabling a given panel on which it is fastened, to remain attached to another panel that includes a layer of interlocking material that positionally opposes the layer on the given panel. Alternatively, an attachment segment may be defined as a surface of the vest 200 or of the belt 1200 that is formed from (i.e. is itself a layer of) an interlocking material.

It will be understood that an interlocking material may include any type of material provided on or as a given surface that, with respect to another surface provided with the same material or different material, is capable of: (A) remaining attached to the other surface without the aid of external force; and (B) being repeatedly detached from and reattached to the other surface. As discussed herein, a same material includes materials having corresponding components, as in the case of hook and loop fastener material wherein a layer component for a given surface includes hooks, and an opposing layer component for another surface includes loops.

As illustrated in FIGS. 2A and 2B, the head 250 extends from the upper edge 234 to the transition 258 between the head 250 and the body 252. The body 252 extends from the transition 258 to the lower end 208 of the vest 100. Each of the head 250 and the body 252 encompass respective portions of the front region 202 and the back region 204 of the vest 200. The transition 258 corresponds to lower ends 262a, 266a of the first and second arm access 262, 266. At least two body grips 280 are attached to the exterior surfaces 210a, 220a, 230a of the vest 100 over the body 252. Further, one body grip 280 is attached to the first panel 210 and the back panel 230 along the first side seam 254a, and the other body grip 280 is attached to the second panel 210 and the back panel 230 along the second side seam 254a. Thus, with the configuration illustrated in FIGS. 2A and 2B, the respective attachments of the body grips 280 cross over the first side seam 254a and second side seam 254b and may reinforce respective attachments between the back panel 230 and the first panel 210 and the second panel 220.

As illustrated in FIGS. 2A and 2B, the vest 200 is provided with two body grips 280 extending coaxially with respective side seams 254a, 254b. It will be understood the vest 200 may include two or more body grips 280, and the body grips 280 may be orientated at different angles. For example, one body grip 280 may be angled to have an end thereof attached to the second panel 220 of the vest 200 illustrated in FIGS. 2A and 2B, and an opposite end thereof attached to the back panel 230. Further, a given body grip 280 may be entirely attached to an area illustrated in FIGS. 2A and 2B that corresponds to a respective one of the first panel 210, the second panel 220, or the back panel 230. This configuration is especially possible in the case where first panel 210 or the second panel 220 is formed as one piece with the back panel 230 at least in the area of, and therefore does not include, the first side seam 254a or the second side seam 254b.

It will be understood that the number, placement, and orientation of one or more of the grips 218, 228, 236, 238, 280 may be customized to a given patient or caregiver in order to: optimize the comfort of the patient; allow the caregiver to achieve an optimal mechanical advantage given the caregiver's respective size and strength; and reduce the risk of injury to the patient and caregiver when, for example, the caregiver is transferring the patient from a bed, such as the bed 30 in FIGS. 1A and 1B, to another location, bed, chair, wheelchair, piece of physical therapy equipment, etc. The flexibility in placements, sizes, and/or orientations of the grips 218, 228, 236, 238, 280 is enabled by the aspects of the vest compression system 100 that cause the vest 200 to remain substantially in place relative to a body of a patient when external forces are applied to the grips.

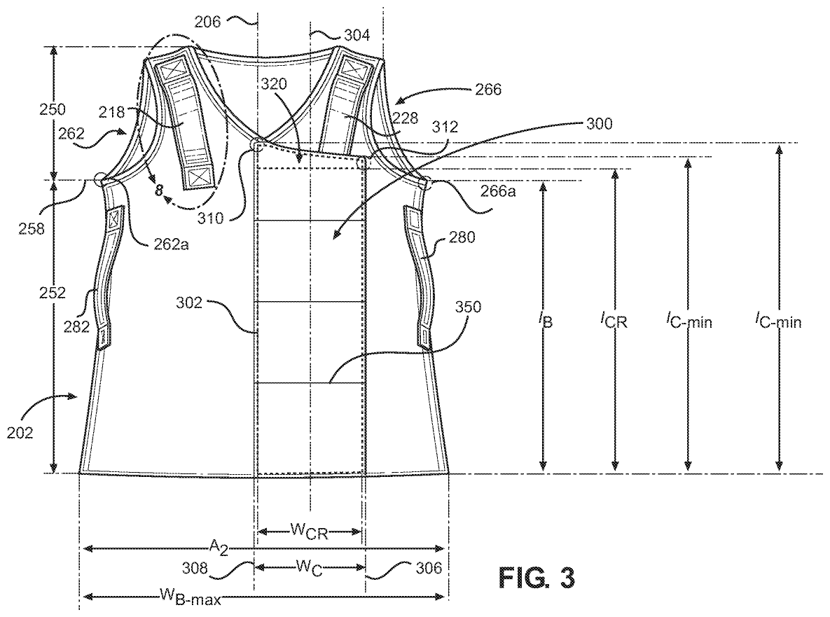

FIG. 3 illustrates a front elevation view of the vest 100 in a closed state, according to an aspect of the present disclosure. In particular, the vest 100 is shown in a flattened state to illustrate an entirety of the front region 202. As illustrated in FIG. 3, the body 252 has a maximum width w_{B-max} (“max body width w_{B-max}”) that is measured: at the lower end 208 of the vest 200; from the first side seam 254a (or area of the vest 100 corresponding thereto) to the second side seam 254b (or area of the vest 100 corresponding thereto); and with the vest 200 in the flattened state. The max body width w_{B-max} also corresponding to a maximum width of the vest 200. Further, the head 250 has a maximum width w_{H-max} (“max head width w_{H-max}”) measured at the transition 258 from the first side seam 254a (or area of the vest 100 corresponding thereto) to the second side seam 254b (or area of the vest 100 corresponding thereto). Further a length I_{B} of the body 252 (“body length I_{B}”) is measured from the transition 258 to the lower end 208 of the vest 200.

As illustrated in FIG. 3, the vest 200 includes a vest closure 300 that is disposed within the front region 202, and extends at least over a closure region 302. A second longitudinal axis 304 of the closure 300 and closure region 302 may be offset from, and extend parallel to, the first longitudinal axis 206 as shown in FIG. 3. As further illustrated in FIG. 3, the closure 300 has a width w_{C} (“closure width w_{C}”) defined as a distance between a leading edge 306 and a trailing edge 308 thereof. The leading edge 306 and the trailing edge 308 correspond to the outer-most boundaries within a plane of a page of FIG. 3, where first and second front attachment segments 402, 412, or portions thereof, may be positioned on the first panel 210 and the second panel 220 respectively, as discussed in more detail with reference to FIG. 4.

For any of the configurations of the closure 300 described herein, the closure width w_{C} is at least equal to the closure region width w_{CR}; and the closure region width w_{CR} is equal to at least one fourth (¼) of the max body width w_{B-max}. The closure width w_{C} illustrated in FIG. 3 is slightly greater than the closure region width w_{CR}, and may be even greater according to aspects of the present disclosure.

The closure 300 may be configured to have a maximum length I_{C-max} (“max closure length I_{C-max}”) and a minimum length I_{C-min} (“minimum closure length I_{C-min}”). The closure 300 illustrated in FIGS. 3 and 4 has a respective maximum length I_{C-max} measured from the lower end 208 to an initial attachment point 310 of the upper lip 268 to the first panel 210; and a respective minimum closure length I_{C-min} measured from the lower end 208 to a corner 312 of the first inner edge 214. As illustrated in FIG. 3, both the max closure length I_{C-max} and the minimum closure length I_{C-min} is greater than a length I_{CR} of the closure region (“closure region length I_{CR}”). The closure region length I_{CR} is defined as a length of the closure region 302 extending along the second longitudinal axis 304. Alternatively, the closure may have a flat upper edge and therefore only have a single closure length I_{C} (not shown) measured from the lower end 208 to the flat upper edge. For any of the configurations of the closure 300 described herein, the minimum closure length I_{C-min} or the single closure length I_{C} is at least equal to the closure region length I_{CR}, which is at least equal to the body length I_{B}.

A sub-area of the closure 300 between the initial attachment point 310, the corner 312, and the transition 258 defines a collar closure 320. The collar closure 320 corresponds to an upper-most end of the closure 300 and is part of one exemplary configuration of the closure 300 that provides for first and second front attachment segments 402, 412 along every portion of the first inner edge 214 not including the upper lip 268. The collar closure 320 ensures that when a vest 200 is worn by a patient, a portion of the first panel 210 closest to the patient's face remains firmly attached to the second panel 220 for the patient's comfort. In configurations of the vest 200 having a single closure length I_{C} equal to the body length I_{B} (and thus equal to the closure region length I_{CR}), the collar closure 320 is accordingly always incorporated. According to an aspect of the present disclosure, in configurations of the vest 200 having a minimum closure length I_{C-min} equal to or greater than the body length I_{B}, or a single closure length I_{C} that is greater than the body length I_{B}, the collar closure 320 may be provided as part of a configuration desired by a patient or a caregiver.

According to an aspect of the present disclosure, the first and second front attachment segments 402, 412 referred to above and discussed in more detail with reference to FIG. 4, may be attached to respective panels by segment stitching 350 illustrated in FIG. 3. The segment stitching 350 may be provided so as to pass through: (A) a given panel; and (B) an attachment segment in the form of a layer secured to the given panel.

FIG. 4 illustrates a front elevation view of the vest 100 in a first open state, according to an aspect of the present disclosure. In particular, the first panel 210 is shown as being flipped over so as to expose at least a portion of the first interior surface 210b that includes a first front attachment segment 402 of a first flap 400 of the first panel 210. In FIG. 4, at least that portion of the second exterior surface 220a of the second panel 220 that is overlapped in FIG. 3 by the first panel 210 is shown. Said portion including a second front attachment segment 412 of a second flap 410 of the second panel 220.

FIG. 4 further illustrates segment stitching 350 that attaches the second back attachment segment 242 to the back panel 230, and a third back attachment segment 420 that may be provided on or as part of the back interior surface 230b as shown in FIG. 4. According to an aspect of the present disclosure, the third back attachment segment 420 may be a layer type of attachment segment as discussed above, and attached to the back panel 230 by double segment stitching 450. The double segment stitching 450 may be constituted by stitching or reinforcement stitching that extends through the third back attachment segment 420, the back panel 230, and the first back attachment segment 240 to securely bind the three layers together.

With further reference to FIGS. 3 and 4, the first flap 400 and the second flap 410 define the closure 300 of the vest 200. More specifically, the first flap 400 and the second flap 410 include at least those portions of the first panel 210 and the second panel 220 disposed between the leading edge 306 and the trailing edge 308 illustrated in FIG. 3. A configuration of portions of the first front attachment segment(s) 402 configured to mirror and attach to portions of the second front attachment segment(s) 412 (or vice versa) in a closed state of the vest 200 defines an attachment configuration of the closure 300.

According to an exemplary aspect of the present disclosure, the first and second attachment segments 402, 412 may be provided such that a position(s) and configuration(s) of the first front attachment segment(s) 402 on, or as part of, the first interior surface 210b for the first flap 402, may correspond (e.g. mirror, be matched) exactly to a position(s) and configuration(s) of the second front attachment segment(s) 412 on, or as part of, the second exterior 220a for the second flap 412 in the closed state of the vest 200. For such a configuration, a value of an area of the attachment configuration is equal to an aggregate of all of the respective areas (single continuous or separate segments) of the first front attachment segment 402, which is the same as an aggregate of all of the respective areas of the second front attachment segment(s) 412.

According to another exemplary aspect of the present disclosure, the first and second attachment segments 402, 412 may be provided such that: a number of separate first front attachment segments 402 is fewer than a number of separate second front attachment segments 412 (or vice versa); or a single first front attachment segment 402 is smaller than a single second front attachment segment 412 (or vice versa). In such a configuration, the vest 200 may have multiple closed states for which: all of the separate first front attachment segments 402 are attached to some of the separate second front attachment segments 412; or an entirety of the single first front attachment segment 402 is attached to a portion of the single second front attachment segment 412. As a result, a size of a person that the vest 200 can accommodate may be different for each of the multiple closed states. For these configurations, an area of the attachment configuration is defined as: an aggregate of respective areas of the separate attachment segments on the one of the first and second attachment segments 402, 412 having the lesser number of separate attachment segments; or an area of the smaller of the single first front attachment segment 402 and the single second front attachment segments 412.

In FIG. 4, the closure region 302 and second longitudinal axis 304 are represented relative to each of the first flap 400 and the second flap 410, for the purposes of explaining a relative total size of the area of the attachment configuration of the closure 300. According to an aspect of the present disclosure, the first front attachment segment 402 may extend over an entirety of the first flap 400, and the second front attachment segment 412 may span over an entirety of the second flap 410 as shown in FIG. 4. Thus, at least an entirety of the first interior surface 210b corresponding to the closure region 302 includes the first front attachment segment 402. Likewise, at least an entirety of the second exterior surface 220a corresponding to the closure region 302 includes the second front attachment segment 412. As a result, an area of the attachment configuration of the closure 300 illustrated in FIG. 4 is greater than an area of the closure region 302, and thus greater than a value of the body length I_{B} multiplied by one fourth (¼) of the max body width w_{B-max} (i.e., the area of the attachment configuration exceeds the value of (I_{B}×(¼)w_{B-max})).

As discussed above with respect to the vest 200 illustrated in FIGS. 3 and 4, the area of the attachment configuration provided by the first and second front attachment segments 402, 412 is greater than the area of the closure region 302 which is equal to:

(I_{B}×(¼)w_{B-max}).

According to an aspect of the present disclosure, for other configurations of the vest 200, a value of the area of the attachment configuration of the closure 300 may be less than 100%, but is at least 66%, of a value of the area of the closure region 302. Accordingly, the area of the attachment configuration is at least equal to:

(⅔)×(I_{B}×(¼)w_{B-max}).

This minimum size of the area of the attachment configuration ensures that an implementation of the closure 300 results in the vest 200 remaining closed and stationary relative to a patient when any of the grips are utilized to lift, support, guide, catch, and/or move the patient.

According to an aspect of the present disclosure, to place the vest 200 on a patient, for example, the vest 200 is opened by detaching the first flap 400 from the second flap 410 (thereby opening the closure 300). More specifically, the first front attachment segment 402 is detached from the second front attachment segment 412. Once positioned on the patient, the first panel 210 is positioned over (wrapped on to) the second panel 220 such that the first front attachment segment 402 is attached to the second front attachment segment 412. In particular, the first panel 210 is preferably positioned so that the first front attachment segment 402 mirrors the second front attachment segment 412 and the attachment configuration of the closure 300 is fully implemented. Full implementation of the attachment arrangement ensures any potential movement of the vest 200 relative to a patient is substantially limited if not entirely prevented.

A continuous attachment configuration, for example as shown in FIG. 4, prevents the vest 200 from bunching up when grips 218, 228, 236, 238, 280 are being used as discussed herein. This may also be achieved with specific sizing and positioning of respective spaced apart segments of the first and second front attachment segments 402, 412, so as to meet the minimum area requirement for the attachment configuration discussed herein. For example, with reference to the vest 200 illustrated in FIGS. 3 and 4, each of first and second front attachment segments 402, 412 of another exemplary attachment configuration according to the present disclosure may include separate attachment segments that are spaced apart along the second longitudinal axis 304. More specifically, the separate attachment segments may be positioned at least in locations corresponding to top, middle, and bottom portions of the closure region 302. Further, each of the separate attachment segments may extend perpendicular to the second longitudinal axis 304 continuously over an entire width of the closure region 302. Thus, portions of the first and second front attachment segments 402, 412 may be provided at least at the lower end 208, the transition 258, and an in location there between. According to an aspect of the present disclosure, locations for portions of the first and second front attachment segments 402, 412 may be limited to being positioned within an area having a width equal to one fourth (¼) of the max body width w_{B-max}.

With reference to FIG. 3, the second longitudinal axis 304 of the closure 300 and closure region 302 is offset from the first longitudinal axis 206 of the vest 200. Thus, unlike a garment that opens and closes via a zipper or hook and loop fastening centered on the garment, the first flap 400 is wrapped or positioned past a center of the vest 200 to close the vest 200. This is a result of a size (width) of the first panel 210 and provides a large area to grab a hold of when the vest 200 is being put on a patient. This larger area allows the patient or person putting vest 200 on the patient to obtain a firm grip of the first panel 210 and wrap the vest 200 tightly, and maximize a compressive effect of the vest 200, on the patient. This in turn maximizes an ability of the vest 200 to remain stationary relative to the patient when the grips are used to lift, support, guide, catch, or move the patient.

It will be understood that a location where a garment is closed, especially a form-fitting garment, corresponds to a locus of force or pressure that is continuously applied on an individual wearing the garment. With the offset configuration of the closure 300 of the vest 200 according to the present disclosure, a primary area where the vest 200 is closed does not coincide with a center of the patient's chest and stomach, and is not disposed in a centered manner directly below the patient's neck. For many patients these areas may be more sensitive than other parts of the body, and the offset configuration of the closure 300 may be more comfortable than centered configurations because the locus of force or pressure that is continuously applied to a patient by the closure 300 is distanced from these areas. In addition, in the rare case where the vest 200 may not remain stationary relative to the patient, the positioning of the closure 300 ensures that the patient will not be at risk of an inflexible object, such as a zipper or a middle portion of a hook and loop fastening that may include a total of four material layers, being pushed against the patient's neck.

A resistance to opening due to a balance of forces is a further advantage of the exemplary offset configuration of the closure 300. A location where a garment is closed, especially a form-fitting garment, also corresponds to a locus of force or pressure that is continuously applied on the garment by an individual wearing the garment. In a situation where a caregiver is supporting a patient's weight by holding on to, for example, opposite sides of the patient's garment, a direction (vector) of a force of patient's weight is likely to be aligned with and exerted in a direction corresponding to the center of the patient. Accordingly, the force of the patient's weight would be exerted directly on a centered closure, in addition to the continuous force already being exerted on the closure by the patient. According to the present disclosure, the closure 300 of the vest 200 is more proximate to where a force(s) may be applied to the second front grip 228 or closed body grip 280, than a center of the vest 200. Said force counteracts a force of a person's weight on the vest 200 that the closure 300 must be able to withstand for the vest 200 to remain closed. This, plus the condition of a force of the patient's weight being directly exerted on a location of the vest 200 that is offset from a substantial area of the closure 300 and offset from where the patient exerts a continuous force on the closure, reduces the force the closure 300 must withstand for the vest 200 to remain closed in comparison to a centered configuration. Thus, the offset configuration enhances an ability of the closure 300 to remain closed.

According to an aspect of the present disclosure, the closure 300 and accompanying closure region may be orientated such that the second longitudinal axis 304 intersects the first longitudinal axis 206. In particular, a point of intersection may be at location corresponding to a bottom point of the collar 270 or a point along the lower end 208 of the vest. In such a configuration, the first inner edge 214 may be angled so that a portion thereof below the transition extends parallel to the second longitudinal axis 304 intersects. In such a configuration, the length of the closure region 302 for the purposes of providing an attachment configuration of the first and second front attachment segments 402, 412 according to the minimum area defined herein extends along the second longitudinal axis 304. According to another aspect of the present disclosure, the vest 200 may include a front region that may be defined a continuous panel similar to the back panel 230 of the vest 200 illustrated in FIGS. 2-4, and a back region may be defined by a first panel and a second back that define a closure similar to the closure 300.

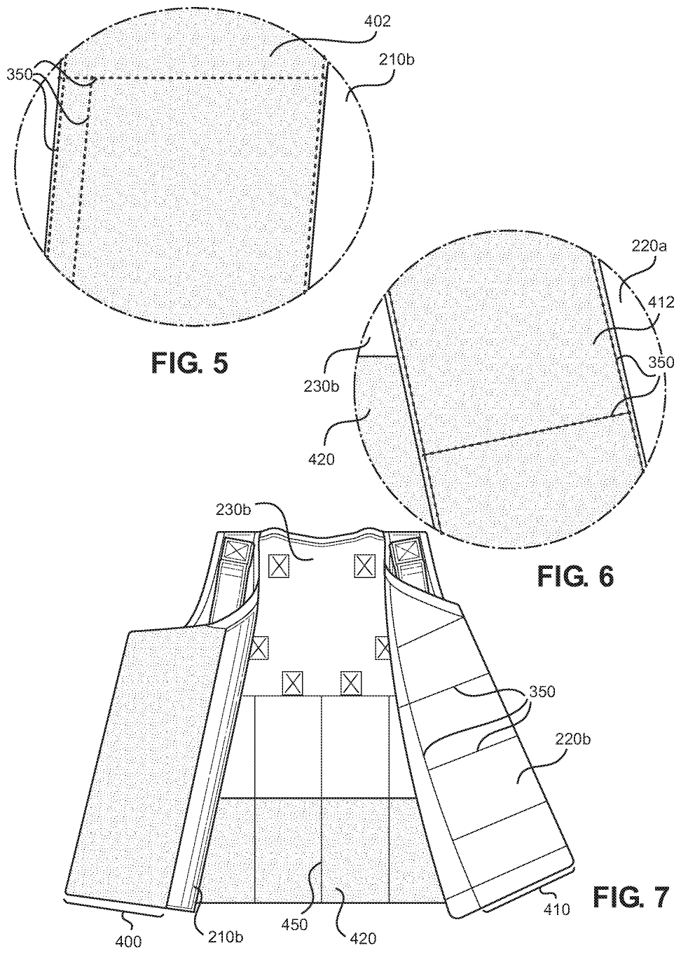

FIG. 5 illustrates an enlarged portion of FIG. 4, identified as Detail 5. According to an aspect of the present disclosure, the first and second front attachment segments 402, 412 are provided as layers fastened to the first and second panels 210, 220 as previously described. With further reference to FIGS. 3-5, the segment stitching 350 for the first panel 210 may be visible on the first exterior surface 210a as shown in FIG. 3, and substantially visible on the first front attachment segment 402 as shown in FIG. 5. A pattern of the segment stitching 350 used to secure the first front attachment segment 402 to the first interior surface 210b, may follow the outer edges of the first front attachment segment 402, as well as fully traverse a width of the first front attachment segment 402 at several locations along the second longitudinal axis 304.

FIG. 6 illustrates an enlarged portion of FIG. 4, identified as Detail 6. Similar to the first front attachment segment 402, the second front attachment segment 412 is secured to the second exterior surface 220a by respective segment stitching 350 that follows along the outer edges, and in some locations fully traverses an entire width, of the second front attachment segment 410. According to an aspect of the present disclosure, the segment stitching 350 may be substantially visible on the second front attachment segment 412 as it passes through both the second panel 220 and the second front attachment segment 412.

FIG. 7 illustrates an elevation view of a front of the vest 200 in a second open state, according to an aspect of the present disclosure. In particular, the second panel 220 is shown as being flipped over so as to expose at least a portion of the second interior surface 220b that corresponds to the second flap 410, and therefore corresponds to at least a portion of the second exterior surface 220a that includes the second front attachment segment 402. As illustrated in FIG. 7, the segment stitching 350 for the second front attachment segment 412 may pass through and be visible on the second interior surface 220b.

Patterns of the segment stitching 350 used to secure the first and second front attachment segments 402, 412 respectively to the first interior surface 210b and the second exterior surface 220a are illustrated in FIGS. 3-7. As discussed above, these patterns may follow the outer edges of a respective attachment segment as well as fully traverse a respective width thereof at several locations along the second longitudinal axis 304 as represented in FIG. 4. A pattern of the segment stitching 350 for the first front attachment segment 402 may substantially mirror the pattern for the segment stitching 350 for the second front attachment segment 412.

However, one of ordinary skill in the art will recognize that other stitching patterns (e.g. outer edges plus crossing patterns, more lines extending along a length direction, crossing patterns along the length direction, etc.) may be utilized to ensure an integrity of the attachments between the first and second panels 210, 220 and the first and second front attachment segments 402, 412 is maintained through each of a multitude of uses of the vest 200. Further, it will be understood that a pattern for the first front attachment segment 402 may be different than a pattern utilized to secure the second front attachment segment. According to an aspect of the present disclosure, other types of stitching or fastening arrangements that do not require a stitching or other fastening material to entirely pass through portions of the first or second panels 210, 220 and/or the first and second front attachment segments 410, 412, may be utilized.

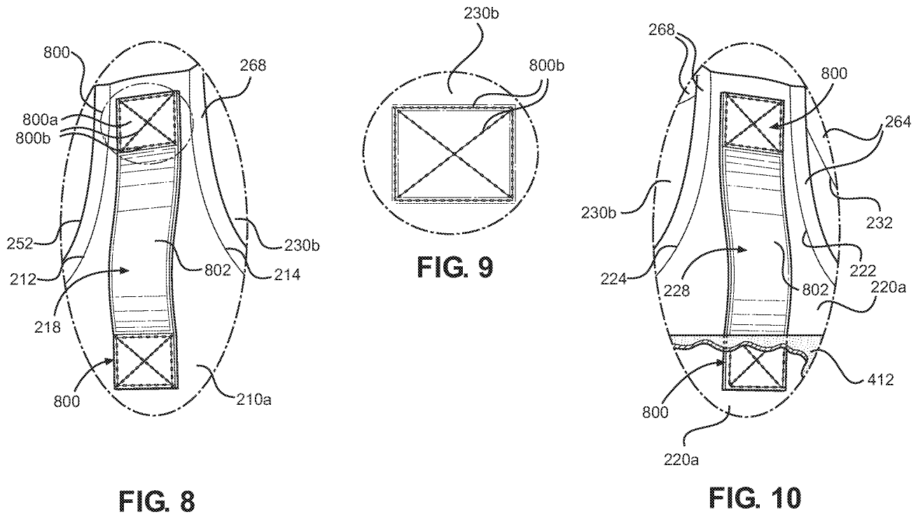

FIG. 8 illustrates an enlarged portion of FIG. 3, identified as Detail 8. In particular, the first front grip 218 and respective grip attachments 800 are shown in FIG. 8. Each of the grips discussed herein includes at least two grip attachments 800 of the type illustrated in FIGS. 8 and 9, and a grip body 802 extending between the grip attachments. Each grip attachment 800 includes an end 800a of a respective grip (“grip end 800a”), in this case first front grip 218, and grip stitching 800b. As illustrated in FIG. 8, an upper grip end 800a of the first front grip 218 is attached to the first exterior surface 210a in a position below the first upper edge 216 and between the first outer edge 212 inside of the first outer lip 260 and the first inner edge 214 inside of the upper lip 268. A lower grip end 800a may be attached to the first exterior surface 210a at, above, or below the transition 258.

The grip stitching 800b may be different from the segment stitching 350 in that the grip stitching 800b encompasses one or more types of reinforcement stitching (e.g., backstitching, bartacking, triple stitching), such that there may be more stitches per inch than that of the segment stitching 350. The increased stitches per inch provides a more secure attachment between, in this case, the first front grip 218 and the vest 200, because the greatest magnitude of force will be applied to the grips during a usage of the compression vest system 100 in, for example, the implementation illustrated in FIG. 1B. Accordingly, locations where the grips are attached to the vest 200 or the belt 1200 will be subject to more strain and could become potential points of failure, and therefore must be secured to a heightened degree.

It will be understood that the segment stitching 350 may also constitute reinforcement stitching. However, whereas a level of securement (strength of attachment) provided by this type of stitching may correspond to a maximum attachment strength for an attachment segment, it is a minimum threshold for attachment strength associated with a grip according the present disclosure. More generally, each grip of the compression vest system 100 will be attached to a respective component of the system (vest 200, belt 1200) by a stitching that provides at least the same level of securement as provided by reinforcement stitching.

In addition to the grip attachments 800, each of the grips discussed herein may have a grip body 802 that is separated from a surface of the vest over which it traverses and defines a component of the grip that may be grasped by a patient or caregiver.

FIG. 9 illustrates an enlarged portion of FIG. 4, identified as Detail 9. More specifically, FIG. 9 illustrates the grip stitching 800b for one of the first back grips 236. The grip stitching 800b as provided on the back interior surface 230b is the result of providing reinforcement stitching that passes through a respective grip end 800a and a respective one of the first, second, or back panel 210, 220, 230. As illustrated in FIG. 9, the grip stitching 800b passes through the back panel 230 and is patterned on to the back interior surface 230b.

FIG. 10 illustrates an enlarged portion of FIG. 4, identified as Detail 10. In particular, the second front grip 228 and respective grip attachments 800 are shown in FIG. 10. The second front grip 228 has substantially the same construction as the first front 218. Like the first front grip 218, the grip ends 800a of the second front grip 228 are attached to the second exterior surface 220b by grip stitching 800b. More specifically, an upper grip end 800a of the second front grip 228 is attached by grip stitching 800b to the second exterior surface 220a in a position below the second upper edge 226 and between the second outer edge 222 inside of the second outer lip 264 and the second inner edge 224 inside of the upper lip 268.

A difference between the first front grip 218 and the second front grip 228 is that for a given configuration of the closure 300, the second front attachment segment 412 may extend above the transition 258, and a lower grip end 800a of the second front grip 228 may be attached to the first exterior surface 210a at or below the transition 258. Thus, the lower grip end 800 of the second front grip 228 may be covered by the second front attachment segment 412. A portion of the second front attachment segment 228 covering the lower grip attachment 800 is cut away in FIG. 10 so that the lower grip attachment 800 is visible. As a result, a portion of an upper edge of the second front attachment segment 228 (where the second front attachment segment 412 is provided as a layer) may be attached to the second front grip 228 via segment stitching 350, or to the second panel 220 and the second front grip 228 via double segment stitching 450. Alternatively, the second front attachment segment 412 may have a cutout section to accommodate the lower grip end 800a of the second front grip 228, and the lower grip end 800a may include a respective attachment segment secured thereto or formed as a part thereof.

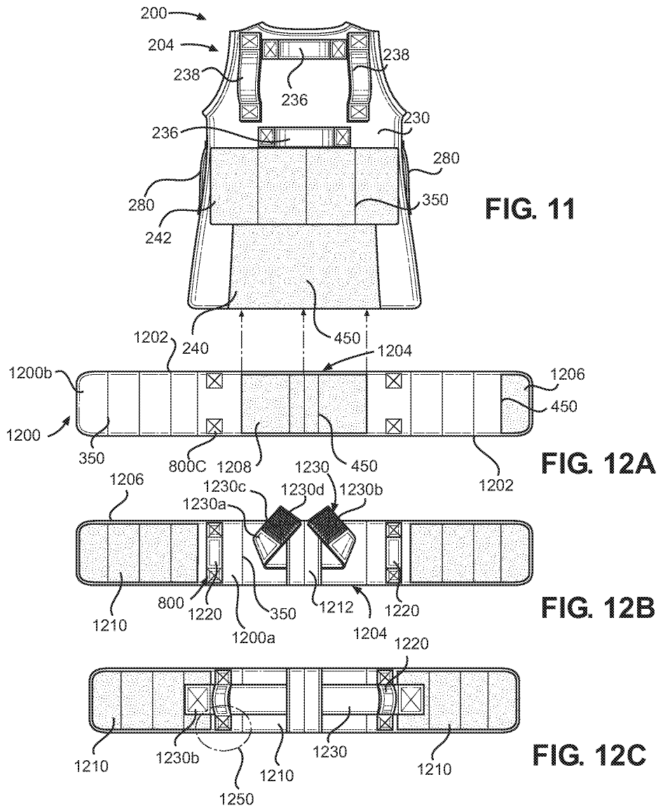

FIG. 11 illustrates an elevation view of the back region 204 of the vest 200, according to an aspect of the present disclosure. In particular, FIG. 11 illustrates the back region 204 with the vest 200 in a flattened state and thereby illustrates the relative sizes of the first and second back attachment segments 240, 242. According to an aspect of the present disclosure, the first back attachment segment 240 may extend for over half of the max body width w_{B-max}, and the second back attachment segment 242 may traverse a substantial entirety of a width the vest 200 between the body grips 280 where it is provided. Each of the first and second back attachment segments 240, 242 may be provided as rectangular in shape. However, it will be understood that the first and second back attachment segments 240, 242 may be provided in other shapes and sizes. FIG. 11 further illustrates the double segment stitching 450 that attaches the first back attachment segment 240 and the third back attachment segment 420 (FIG. 4) to the back panel 230.

FIG. 12A illustrates an elevation view of an interior surface 1200b of the belt 1200 of the compression vest system 100 in a passive state, according to an aspect of the present disclosure. The belt 1200 includes two belt flanks 1202 that extend from opposite sides of a middle portion 1204 of the belt. A first belt attachment segment 1206 may be provided on a far end of one belt flank 1202, and may be fastened to the belt 1200 via double segment stitching 450. A second belt attachment segment 1208 may be provided over the middle portion 1204 of the belt 1200, and thus between the belt flanks 1202. The second belt attachment segment 1208 may be fastened to the belt 1200 by both segment stitching 350 and double segment stitching 450. The second belt attachment segment 1208 is configured to detachably attach to at least the first back attachment segment 240. Accordingly, a shape and size of the second belt attachment segment 1208 may substantially correspond to a shape and size of the first back attachment segment 240. According to another aspect of the present disclosure the first back attachment segment 240 may be larger than the second belt attachment segment 1208 so as to promote the belt 1200 remaining on, and stationary relative to, the vest 200.

FIG. 12B illustrates an elevation view of an exterior surface 1200a of the belt 1200 of the compression system 100 in a passive state, according to an aspect of the present disclosure. Third back attachment segments 1210 may be fastened to the belt flanks 1202 of the belt 1200 by segment stitching 350, with the exception of a portion of one of the third back attachment segments 1210 that corresponds to the first belt attachment segment 1202 where double segment stitching 450 may be incorporated. The belt 1200 further includes a reinforcement segment 1212 provided in a central area of the middle portion 1204 of the belt 1200. At least one belt grip 1220 may be attached to the belt exterior surface 1200a on each side of the middle portion 1204 via respective grip attachments 800. Similar to grips provided on the vest 200, the grip attachments 800 include grip ends 800a attached to the belt exterior surface 1200a by grip stitching 800b that may be visible on the belt interior surface (FIG. 12B).

The reinforcement segment 1212 covers, and may provide attachment points for, one or more elastic tension bands 1230. Each elastic tension bands 1230 includes a band body 1230a and a band end 1230b extending from the band body 1230a. An attachment segment 1230c (hereafter referred to as “band attachment segment 1230c”) may be attached to the band end 1230b by reinforcement-type stitching 1230d (hereafter referred to as “band stitching 1230d”) similar to reinforcement stitching of the grip stitching 800b.

FIGS. 12A and 12B illustrate the belt 1200 in the passive state that results from the attachment segments 1230c being detached from the third belt attachment segments 1210. In use, the band ends 1230b may be passed through spaces between respective belt grips 1220 and the exterior surface 1200a, and the band attachment segments 1230c may be attached to respective third belt attachment segments 1210. If both elastic tension bands 1230 are not extended beyond respective normal state lengths prior to attachment to the third belt attachment segments 1210, the belt 1200 will remain in the passive state.

FIG. 12C illustrates an elevation view of the exterior belt surface 1200a of the belt 1200 of the compression system 100 in an active state, according to an aspect of the present disclosure. In particular, both elastic tension bands 1230 are extended beyond respective normal state lengths and attached to respective third belt attachment segments 1210. In FIG. 12C, the belt 1200 is not attached to the vest 200 and the elastic tension bands 1230 cause portions of the belt 1200 between the reinforcement band 1210 and the third belt attachment segments 1210 to recoil. However, if the belt 1200 is attached to the vest 200 via the second belt attachment segment 1208, and one of the first or second back attachment segments 240, 242, the portions of the belt 1200 including the second belt attachment segments 1208 will remain stationary due to the attachment with the first or second back attachment segment 240, 242. Thus, a portion of the belt 1200 capable of recoiling is limited to intermediate portion 1250 between outer edges of the second belt attachment segments 1208 and locations where the band ends 1230b are attached to respective third belt attachment segments 1210.

In either situation where the elastic tension bands 1230 are in a tensile state, and the belt 1200 is thus in an active state, ends of the elastic tension bands 1230 attached to the belt 1200 are in a state of tension. In a situation where the compression vest system 100 is used by a patient that has extended the elastic tension bands 1230 to a maximum length, substantial tensile forces will be exerted on attachment points between the elastic tension bands 1230 and the belt 1200. As such, double segment stitching 450 may be incorporated to attach a center of the reinforcement strip 1210, ends of the elastic tension bands 1230, and a center of the second belt attachment segment 1204 to the belt interior and exterior surfaces 1200a, 1200b. In addition, edges of the reinforcement band 1210 and corresponding portions of the elastic tension bands 1230 and the second belt attachment segment 1204 may be secured to the belt 1200 with double segment stitching 450 as illustrated in FIGS. 12A-C. Reinforcement type stitching may also be used in order to maintain the integrity of the construction of the belt 1200, especially in locations where the elastic tension bands 1230 are attached thereto.

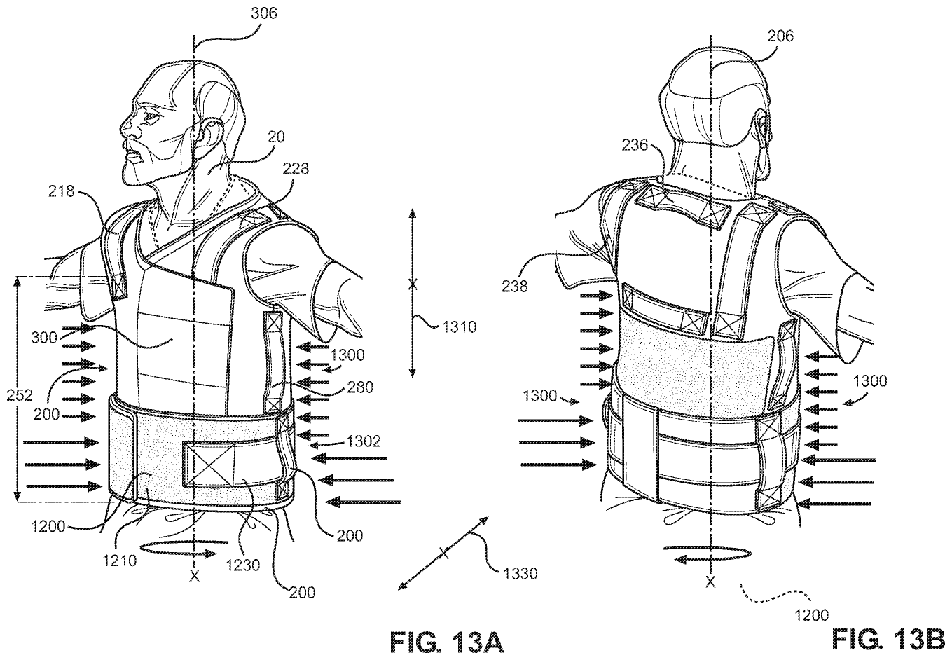

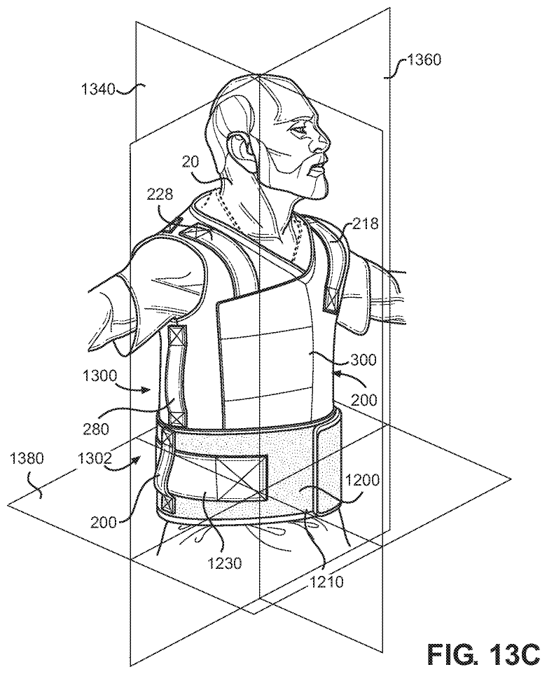

FIGS. 13A and 13B illustrate front and rear perspective views of the compression vest system 100, according to an aspect of the present disclosure. In the use and configuration illustrated in FIGS. 13A and 13B: (1) the first panel 210 is attached to the second panel 220 via the closure 300; (2) the belt 1200 is attached to the vest 200 via the second belt attachment segment 1208 and the second back attachment segments 242; and (3) the belt flanks 1202 are wrapped around a torso of the patient 20 such that the first belt attachment segment 1206 (not shown) is attached to the third belt attachment segment 1210 of an opposite the belt flank 1202. In addition, the elastic tension bands 1230 have been attached to the belt flanks 1202 so as to be in a state of tension.

One of ordinary skill in the art will recognize that the first panel 210 may be attached to the second panel 220 so that the first, second, and back panels 210, 220, 230 tightly wrapped around the torso of the patient 20. As a result, the body 252 of the vest 200 will exert primary compressive forces 1300 on the torso of the patient 20. Adding the belt 1200 so as to be tightly or form-fittingly (with respect to a form of the patient 20) wrapped around the vest 200 worn by the patient 20 will result in second compressive forces 1302 exerted on the torso of the patient 20, particularly in an area of a waist of the patient 20. Extending the elastic tension bands 1230 beyond normal state lengths and attaching them to the third belt attachment segments 1210 increases the second compressive forces 1302 exerted on the patient 20.

The first and second compressive forces 1300, 1302 counter act forces applied to the compression system 100 resulting from any of the first and second front, first and second back, body, and belt grips 218, 228, 236, 238, 280, 1220 being pulled or pushed in any direction. As a result, the first and second compressive forces 1300, 1302 act to resist and eliminate, or at least substantially minimize a vertical displacement 1310, a rotational displacement 1320, and a front to back displacement 1330 of the vest 200 and the belt 1200 of the compression vest system 100 relative to the patient 20. More generally, the compression vest system 100 is fitted and applied to the patient 20 so as not to have components or portions thereof shift on the patient 20 when, for example, a caregiver such as the caregiver 20 illustrated in FIG. 1B, grasps and pulls the first front grip 218 and one of the body grips 280 to aid, guide, and support a movement of the patient 20.

The belt 1200 shown in FIGS. 13A and 13B may be utilized as discussed above. In instances where a patient is significantly large, it may be advantageous to attach a second belt 1200 on the vest 200. According to an aspect the present disclosure, the second back attachment segment 242 may be used for an attachment and application of a second belt 1200 with the vest 200.

FIG. 13C illustrates a front perspective view of the compression vest system 100, and sagittal, coronal, and transverse body planes 1340, 1360, 1380. The body planes illustrated are well known in the art as reference planes that may be used to characterize and analyze spinal curvature (posture) and movement of an individual. In FIG. 13C, the sagittal plane 1340 is disposed at the midline (median) and divides a body of the patient 20 into left and right parts that are equal. Accordingly, the sagittal plane 1340 illustrated is the median sagittal plane. In general, a sagittal body plane divides a body into right and left parts, which may be unequal where the plane analyzed is offset from the midline of the body. Where an analyzed sagittal plane is offset to the left or right from the midline, but closer to the midline than an outer edge of the plane (i.e., side of a body in question), it is considered a medial plane. Conversely, where the analyzed plane is closer to the edge of the plane than the midline it is considered a lateral plane. The coronal plane 1360 divides the body into anterior (front or ventral) and posterior (back or dorsal) parts. Finally, the transverse plane 1380 runs horizontally and divides the body into superior (upper/top) and inferior (lower) parts.

Anatomically, a trunk (or torso) for an individual includes their chest, abdomen, and back (including the spine). Trunk control refers an individual's ability to hold their torso upright, and control movement to and from that upright position. Thus, trunk control corresponds to an ability to control the movement of one's torso within the sagittal and coronal planes 1340, 1360. More particularly, trunk control can be described as an ability to hold a center of one's trunk coincident with a line of intersection between the coronal plane 1360 and the median sagittal plane (sagittal plane 1340 in FIG. 13C).

Posture refers to the natural curves—cervical, thoracic, and lumbar—present in an individual's spine. An individual's posture is a reflection of the respective state of strength and flexibility in that individual's muscles and joints associated with their spine, hip, and legs. Alignment refers to how the head, shoulders, spine, hips, knees and ankles relate and line up with each other. As one of ordinary skill would readily understand, hip, knee, and ankle joints balance the natural curves of an individual's spine as the individual moves. Proper alignment of the body may reduce stress on the spine and help an individual maintain beneficial posture. Concurrently, an upright posture enables an individual to more easily maintain proper alignment.

When proper alignment and beneficial posture is achieved, improved muscle function may result, because muscles have to do less work, or work together more efficiently, to accomplish given movements. A holistic effect of reduced stress on muscles during normal movements may contribute to better optimizing motor control of muscles being used, and autonomic functioning of an individual's nervous system. A more specific benefit with respect to the muscles and joints associated with the spine, hip, and legs, is improved trunk control. Conversely, issues related to the spine giving rise to poor posture and alignment inhibit an individual's ability to exercise trunk control.

The first compressive forces 1300 applied by the vest 200 supplement a core strength of the patient 20 and increase the patient's ability to exercise trunk control. More specifically, by applying the first compressive forces 1300 (with or without the belt 1200), the vest 200 limits movement of a spine of the patient 20 in the sagittal plane 1340 and the coronal plane 1360. Thus, in straightening the spine of the patient 20, the vest 200 by itself, provides general spinal support and stability within two of the three body planes, and can promote improved posture. In addition, alignment between trunk and hips of the patient 20 may be improved. This may allow certain muscle groups in the legs and hips of the patient to work together at the same time, and thereby improve overall muscle function. For example, where the trunk and hips are in alignment, muscles in the hips and legs can fire together at the same time as the patient 20 moves from a sitting to a standing position. This lessens the burden on the muscles around the spine to maintain the trunk from shifting to the left or to right of the sagittal plane 1340.

As the second compressive forces 1302 are directed radially inward similar to the first compressive forces 1300, adding the belt 1200 enhances the spinal support and stability provided by the vest 200 within, and relative to the intersection of, the sagittal and coronal planes 1340, 1360. More significantly, where the belt 1200 is attached to first back attachment segment 240, the second compressive forces 1302 are applied in a lumbar region of the spine of the patient 200. This has the effect of locking a trunk and hips of the patient 20 relative to each other and the transverse plane 1380. With the trunk and hips locked in place in proper (or at least improved) spatial alignment relative to the transvers plane 1380, movement and motor control of the muscles in the hips and lower back occur in a natural (or least less inhibited) manner for an individual for which movements involving these muscles are problematic.

Another benefit of the compression vest system 100 is that continued use may result in strengthening and, in effect, retraining of muscles in the trunk, hips, and legs involved in the movements for which functions of those muscles are improved by the system 100. Further, as the compression vest system 100 causes a trunk of a sitting individual to be in an upright position, the muscles around the individual's spine responsible for holding the torso in the upright position will be active to some degree and thereby passively strengthened. Accordingly, even a person wearing compression vest system 100 may be inactive, muscles associated with the spine may be passively strengthened resulting in improved spinal stability and trunk control. To this point, the compression vest system 100 can assist with active core isometrics and passive strengthening, while at the same time, assist with increasing a spine angle and posture to be more upright. This in turn can help with motor control, posture, and body alignment, which play important roles in breathing, speech, eating, and swallowing, among other important functions of the body.

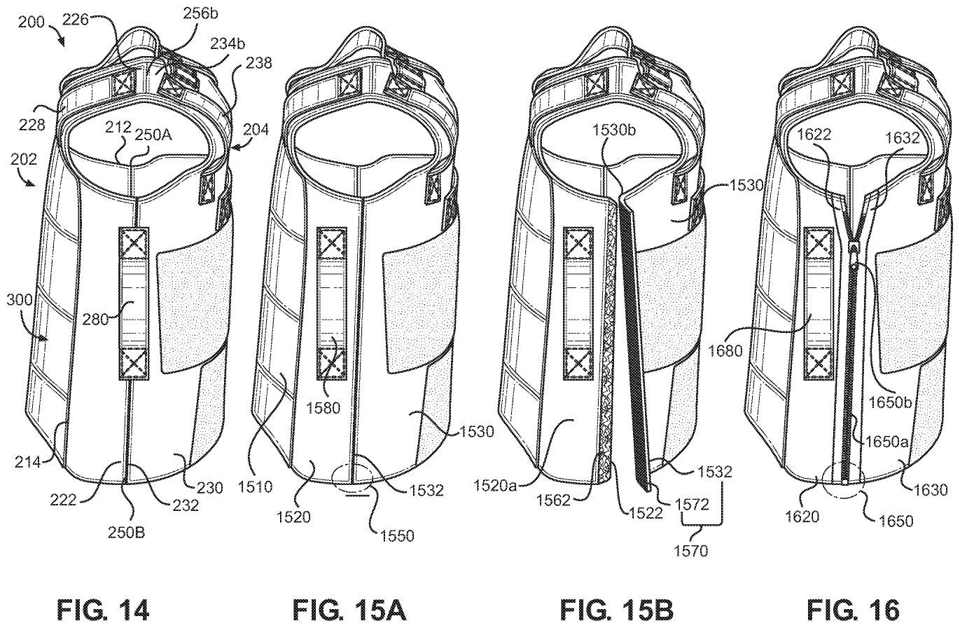

FIG. 14 illustrates a side elevation view of the vest 100, according to an aspect of the present disclosure. In particular, FIG. 14 illustrates a front view of the second side seam 254b where the second outer edge 222 and corresponding back outer edge 232 are attached, as well as the first side seam 254a from and interior perspective of the vest 200. The second upper seam 256b where the second upper edge 226 and corresponding peripheral back upper edge 234b are attached is also shown. Stitching for the first and second side seams 254a, 254b and the first and second upper seam 256a, 256b may include reinforced or other types of stitching. As illustrated in FIG. 14, the body grip 280 attached to the vest 200 over the second side seam 254a includes grip ends 800a attached by grip stitching 800b to both the second panel 220 and the back panel 230. However, other grip attachment configurations as discussed above may be implemented.

FIG. 15A illustrates a side elevation view of a vest 1500 in a closed state, according to an aspect of the present disclosure. The vest 1500 includes an alternative configuration for an attachment between a second panel 1520 and a back panel 1530. Said configuration includes a side closure 1550 defined by a side flap 1560 of the second panel 1520 and a back flap 1570 of the back panel 1530. The back flap 1560 is configured to overlap and attach to the side flap 1560. It will be understood that an attachment between a first panel 1510 and the back panel 1530 may be the same as the attachment with the second panel 1520 or any of the other configurations described herein.

FIG. 15B illustrates a side elevation view of the vest 1500 in an open state, according to an aspect of the present disclosure. More specifically, FIG. 15B illustrates the vest 1500 having a closure 1502 between the first panel 1510 and second panel 1520 being closed, and the side closure 1550 being open. As shown, the side flap 1560 includes a second outer edge 1522 and a side panel attachment segment 1562 provided on or as part of a second exterior surface 1520a of the second panel 1520. The back flap 1570 includes a back outer edge 1532 and a back side attachment segment 1572 attached to at least a portion of a back interior surface 1530b that overlaps the side panel attachment segment 1562 in a closed state. The side panel attachment segment 1562 and the back side attachment segment 1572 may each extend from a lower end 1508 of the vest 1500 to a lower end of an arm access as illustrated in FIG. 15B.

In the configuration illustrated in FIGS. 15A and 15B, a body grip 1580 is provided as being entirely attached to a location on the second panel 1520 so at to be adjacent to the back outer edge 1532 when the side closure 1550 is closed. The positioning of the body grip 1590 is advantageous since the back panel 1530 overlaps the second side panel 1520 for the side closure 1560. Thus, when the body grip 1590 is pulled, a force applied thereto will act to move an overlapped panel (i.e. the second panel 1520) toward an overlapping panel (i.e. the back panel 1530). Accordingly, an attachment between the side panel attachment segment 1562 and the back panel attachment segment 1572 is likely to be enhanced or undisturbed. It will be understood that other configurations may be implemented in which the second panel 1520 overlaps the back panel 1530, and the body grip 1580 is attached to the back panel 1530.

FIG. 16 illustrates a side elevation view of a vest 1600 in a closed state, according to an aspect of the present disclosure. The vest 1600 includes an alternative configuration for an attachment between a second panel 1620 and a back panel 1630 that includes a closure 1650 in the form of a zippered closure. The closure 1650 includes teeth 1650a provided on each of a second outer edge 1622 and a back outer edge 1632, and a zipper slider 1650b. A body grip 1680 is attached to the second side panel 1620 adjacent to the second outer edge 1622, but may be attached to the back panel 1630 adjacent to the back outer edge 1632. Alternatively, each of the second panel 1620 and the back panel 1630 may include a respective body grip 1680 in a respective position discussed above.

The side closure 1550 or the side closure 1650 are particularly advantageous for positioning the vests 1500, 1600 on patients that do not have use of their arms, have had stroke, or suffer from ALS or cerebral palsy. In these situations, a caregiver can open the side closures (1550 or 1650) and place the vest (1500 or 1600) on a patient with little or no help from the patient.

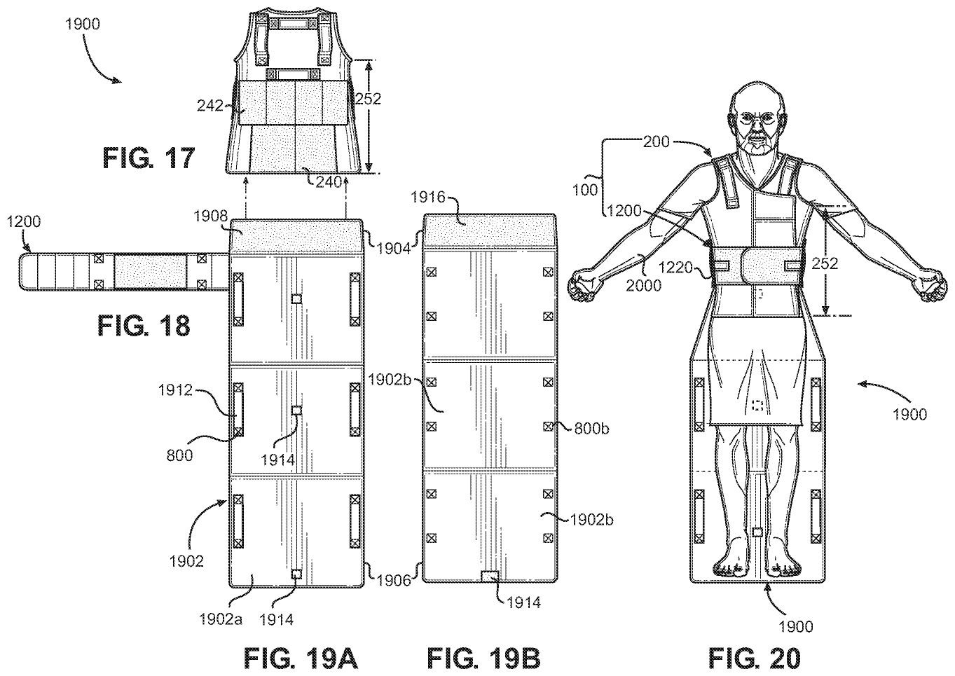

FIGS. 17 and 18 illustrate a compression vest and a compression belt 1200 according to an aspect of the present disclosure. FIGS. 19A and 19B illustrate overhead views of a cloak 1902 according to the present disclosure. Together, FIGS. 17, 18, and 19A (or 19B) illustrate a component view of an integrated compression vest system 1900, according to aspect of the present disclosure. The integrated compression vest system 1900 includes the vest 200, or any of the vests described herein, the belt 1200, and a cloak 1902. The cloak 1902 includes a first end 1904 and a second end 1906. A first surface 1902a of the cloak 1902 is illustrated in FIG. 19A, and includes a first cloak attachment segment 1908 attached or formed on the first end 1904 of the cloak 1902. In addition, cloak grips 1912 are attached to the first surface 1902a along outer edges of the cloak 1902 via respective grip attachments 800. Further, intermediate attachment segments 1914 may be provided along a center of the cloak 1902 as illustrated in FIG. 19A.

FIG. 19B illustrates an overhead view of a second surface 1902b of the cloak 1902, according to aspect of the present disclosure. A second cloak attachment segment 1916 is provided on the second cloak surface 1902b at the first end 1904, and at least one intermediate attachment segment 1914 may be provided at the second end 1906 of the cloak 1902. As shown in FIG. 18, grip stitching 800b extend through and may be visible on the second cloak surface 1902b.

The first and second cloak attachment segments 1908, 1916 may be configured to attach to: (1) the first and/or second back attachment segments 240, 242 of the vest 200; and (2) the second belt attachment segment 1208. The cloak 1902 may be formed from the same or different material (fabric) as the panels 210, 220, 230 of the vest 200. Accordingly, even though the cloak 1902 is illustrated in a flat state in FIGS. 19A and 19B, the cloak 1902 is flexible and can be rolled or folded into a compact arrangement. In addition, the intermediate attachment segments 1914 may include at least one attachment segment positioned on each of the first and second cloak surfaces 1902a, 1902b so as to overlap and attach to one another when the cloak 1902 is rolled or folded into the compact arrangement, and thereby maintain the cloak 1902 in the compact arrangement. At least one intermediate attachment segment 1914 may be positioned so as to be exposed in the compact arrangement such that it may be used to attach the cloak 1902 to, and be carried by, the vest 200 and/or belt 1200 being worn by a patient or carried by a caregiver.

FIG. 20 illustrates an implementation of the integrated compression vest system 1900, according to an aspect of the present disclosure. As in the illustrated implementation, the cloak 1902 may be used for a transport of an individual 1900 that may be immobile. In the particular use of the integrated compression vest system 1900 illustrated in FIGS. 17-19B: (1) the first cloak attachment segment 1908 is attached to the second back attachment segment 242 (see FIG. 17) of the vest 200 worn by the individual 1900; (2) the second belt attachment segment 1208 is attached to the second cloak attachment segments 1916 (See FIG. 18); (3) the belt flanks 1204 are wrapped around the individual 1900; and (4) the elastic tension bands 1230 are stretched past normal state lengths and attached to respective third belt attachment segments 1210. Further with respect to the implementation illustrated in FIG. 18: (5) first compressive forces are applied to the individual 1900 by the vest 200; (6) the cloak 1902 is securely attached to the vest 200 via the first cloak attachment segment 1908 being attached to the second back attachment segment 242, and second compressive force 1302 applied by the belt 1200 to the cloak 1902, the vest 200, and the individual 1900; and (7) the belt 1200 is securely attached to the cloak 1902 as a result of the second cloak attachment segment 1916 being attached to the second belt attachment segment 1208, and the second compressive forces 1302 applied to the cloak 1902 by the belt 1200. Accordingly, in this configuration the cloak 1902 is securely attached to the individual 1900, and with the exception of portions of the cloak 1902 not attached to the vest 200 and the belt 1200 (e.g. portions of the cloak 1902 supporting legs of the individual and having accessible cloak grips 1912), the integrated compression vest system 1900 is configured to remain stationary relative to the individual if external forces are applied to grips 1912 thereof. As such, the front and or back grips 218, 228, 236, 238 on the vest 200, the belt grips 1220, and the cloak grips 1912 may be used (e.g. grabbed, pulled) by a plurality of caregivers to safely lift and carry the individual 2000 similar to a manner in which a stretcher or litter could be used.

According to an aspect of the present disclosure, the first and second cloak attachment segments 1908, 1916 may be provided at a center of the cloak 1902 equidistant from the first cloak end 1904 and the second cloak 1906. Further, cloak grips 1912 may be provided at each of the first cloak end 1904 and the second cloak 1906 such that when the cloak 1902 is attached to the vest 200 being worn by a smaller individual, such as a child, the cloak 1902 extends past a head and legs of the individual. As a result, the cloak 1902 may be used to support the head and legs of the individual where the integrated compression vest system 1900 is used to lift and carry the smaller individual. It will be understood that the cloak may be also be provided in different lengths, and a cloak of a particular size may be selected according to a size of an individual to be supported by the integrated compression vest system 1900.

Additional advantages of the compression vest system 100 described herein are provided by the multiple grips and attachment segments that are accessible from an exterior of the vest (200, 1500, 1600) and the belt 1200. For example, neck braces, back splits, and heat or cool packs may be attached to the back attachment segments 240, 242, 420, or positioned in pockets provided on the interior or exterior surfaces of the vest 200. A multitude of the grips may have rings, such as carabiner clips or D-rings, placed thereon; the rings being capable of being engaged by hooks at the end of an extension of a crane or hoist so that a patient can be hoisted from one location, like a building experiencing an emergency (e.g. fire), to a safe location. Another example of an application of the grips and rings being the transport of an individual from a ship or building via helicopter having a cable(s) attached to the rings attached to the grips on the vest 200. The grips, and specifically the grip attachments described herein 800, being capable of fully supporting the weight of a patient.

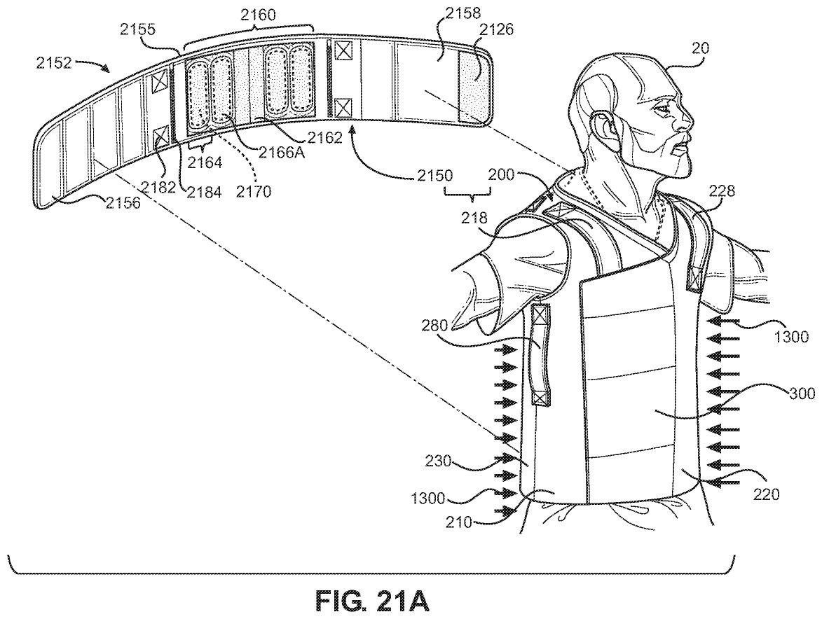

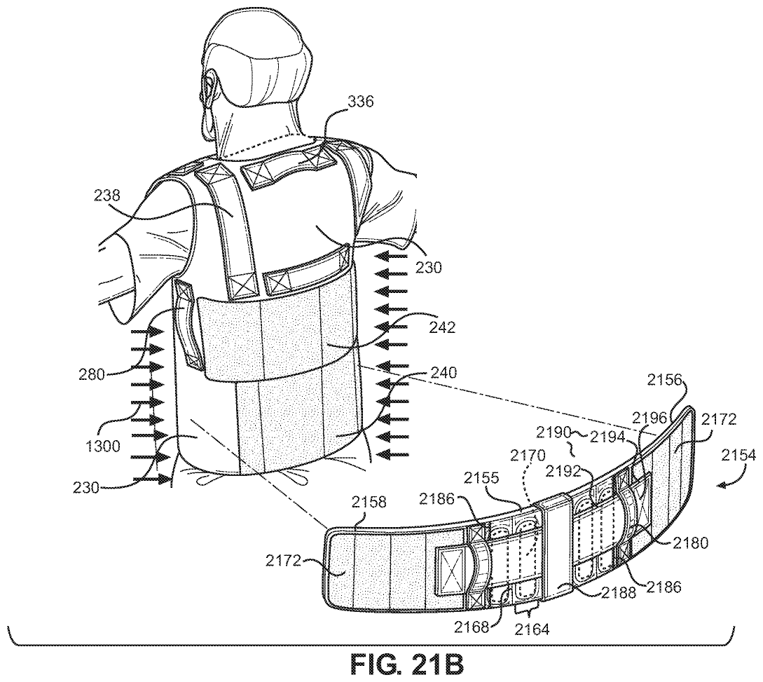

FIGS. 21A and 21B illustrate front and rear perspective views of a compression and lower spinal support vest system 2100 (“compression vest system 2100”) including the compression vest 200 (“vest 200”) and a spinal supportive compression belt 2150 (“belt 2150”).

The vest includes a first panel 210 and a second panel 220; both extending from a back panel 230 that defines a back region. Both of the first and second panels 210, 230 may be respectively attached directly to the back panel via a seam, zippered closure, or hook and loop inter-engagement. One of ordinary skill in the art will recognize that the first panel 210 and/or the second panel 220 may be formed as one piece with the back panel 230. The first, second, and back panels 210, 220, 230 may be formed from the same material or different materials.

According to an aspect of the present disclosure, the first, second, and back panels 210, 220, 230 may be formed from a fabric material such as canvas or nylon. In one example, one or more, or all of the panels 210, 220, 230 may be formed from a material having some degree of water resistance and/or fire retardance, such as a fabric having a fiber thickness of 600 denier (d), 900d, 1000d, or higher to be suitable for an environment in which the compression vest system 2100 will be used. In addition, portions of interior and exterior surfaces of the vest 200 may be lined with cotton or fleece material to provide added comfort to a patient wearing or a caregiver touching the vest 200. Further, portions of the first, second, and back panels 210, 220, 230 may be formed from elastic material.

A closure 300 between the first panel 210 and the second panel 220 is provided by an engagement between an interior surface of the first panel 210 and an exterior surface of the second 220. Accordingly, in a closed state as illustrated in FIG. 21A, the first panel 210 wraps around the patient 20 to overlap the second panel 220. One of ordinary skill in the art will recognize that the first panel 210 may be attached to the second panel 220 so that the first, second, and back panels 210, 220, 230 tightly wrapped around a torso of the patient 20. As a result, the body of the vest 200 will exert primary compressive forces 1300 (“vest compressive force 1300”) radially on the torso of the patient 20. In particular, the first compressive forces 1300 may be applied over an area of the patient's body that corresponds to lower cervical, thoracic, and upper lumbar regions of the patent's spine. Thus, independent of the below described compressive forces applied to the patient by the belt 2150, the vest 200 can apply the first compressive force 1300 to the body (torso) of the patient 20 as a function of the closure 300. The first compressive force 1300, especially in combination with the belt 2150, can prevent the vest 200 from shifting relative to the patient 20 when external forces are applied to grips such as a first grip 218, a body grip 280, front grips 228, first back grips 236, and/or second back grips 238.

The incorporated grips, 218, 228, 236, 238, 280 are substantially inelastic and rigidly attached to a garment (vest 200) that remains stationary relative to the patient 20. As a result, a caregiver, irrespective of a size relative to the patient 20, can, especially when the vest 200 is combined with the belt 2150 which also includes belt grips 2180, select a combination of grips to use that: (A) will maximize a degree of leverage the caregiver is capable of comfortably and safely implementing relative to the patient 20 to aid a movement thereof; and (B) can be relied on to provide a constant grip and minimize a risk of having to re-grip, or dropping the patient 20. As defined herein a patient may be any individual requiring assistance to move in any manner, and a caregiver may be any individual attempting to aid the patient and need not be a medical professional such as a doctor, nurse, or physical therapist.

The vest 200 can be combined with the belt 2150 through an engagement between one of a first back attachment segment 240 and a second back attachment segment 242, and a corresponding central belt attachment segment 2162 described in more detail below. In another example, the belt may be sewn on, or otherwise permanently attached, to the back panel 230 of the vest 200.

As defined herein, an attachment segment includes a layer of interlocking material, such as hook and loop fastener material, that is capable of being fastened (e.g. stitched, glued, molded) to a surface of the vest 200 (i.e. a surface of the first, second, or back panel 210, 220, 230) or the belt 2150. A layer of interlocking material enabling a given panel on which it is fastened, to remain attached to another panel that includes a layer of interlocking material that positionally opposes the layer on the given panel. Alternatively, an attachment segment may be defined as a surface of the vest 200 or of the belt 2150 that is formed from (i.e. is itself a layer of) an interlocking material.

It will be understood that an interlocking material may include any type of material provided on or as a given surface that, with respect to another surface provided with the same material or different material, is capable of: (A) remaining attached to the other surface without the aid of external force; and (B) being repeatedly detached from and reattached to the other surface. As discussed herein, a same material includes materials having corresponding components, as in the case of hook and loop fastener material wherein a layer component for a given surface includes hooks, and an opposing layer component for another surface includes loops.

FIGS. 21A and 21B further illustrate perspective views of an interior 2152 and an exterior 2154 of the belt 2150 (“belt interior 2152”), according to an aspect of the present disclosure. As illustrated in FIG. 21A, the belt 2150 includes a first belt flank 2156 (“first flank 2156) and a second belt flank 2158 (“second flank 2158”) that extend from opposite sides of a central strip 2155 of the belt 2152. An interior attachment segment 2126 may be provided on a far end of the second flank 2158, and may be fastened to the belt 2150 via double segment stitching.

Defined by and between ends of the central strip 2155, is a segmented pouch 2160 that includes a central belt attachment segment 2162. The central belt attachment segment 2162 may be fastened to the belt 2150 by both segment stitching and double segment stitching and be configured to detachably attach to at least the first back attachment segment 240. The central belt attachment segment 2162 can define a surface of the segmented pouch 2160 as well as a portion of a surface of the interior 2152 that spans substantially over a longitudinal length of the central strip 2155. In another example, the central belt attachment segment 2162 can define more or less than an entire surface of the segmented pouch 2160 so as to substantially correspond to a shape and size of the first back attachment segment 240. In another example, the central belt attachment segment 2162 may be shorter than the first back attachment segment 240 so as to promote the belt 2150 remaining on, and stationary relative to, the vest 200.

The segmented pouch 2160 may include one or more pockets 2164 constructed of a first pocket layer 2166A and second pocket layer 2166B (See FIG. 21B) (“layers 2166”) that extend between vertical stitching 2168 (See FIG. 21B). A stay 2170 is provide between the layers 2166 for each pocket 2164 of the segmented pouch 2160. In practice, the stays 2170 will be positioned between an exterior 2154 of the belt 2150 and the back panel 230 of the vest 200.

As defined herein, a stay may be a rigid or semi-rigid type of panel, layer, board, or slat. In one example, a stay may be provided by a flat piece of hard material such as a lightweight metal (e.g., titanium, aluminum, etc.), or a thermoplastic material such as a polycarbonate (e.g, Lexan). In other examples, a stay may be provided by a strip of semi-flexible plastic having a degree of elasticity suitable for the spinal support needs of a particular patient. In another example, a stay may be formed with a degree of curvature suited for a location of a patient's body the stay is configured to support. In yet another example, a stay may be substantially flat along its edges and define a concave profile along a longitudinal length on or both sides. In still another example, a stay may be provided in a shape of a rectangular box having rounded corners and/or beveled edges at least along its width.

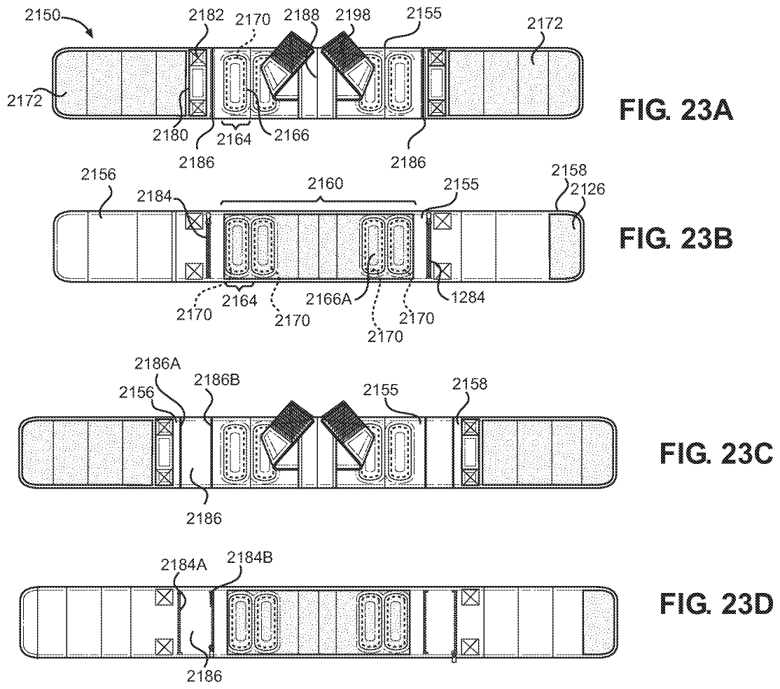

In one example, the central strip 2155 extends between zippers 2184. A tape and chain of each zipper 2184 respectively closest to the segmented pouch 2160 defines an outer edge of the central strip 2155. Concurrently, a tape and chain of each zipper 2184 not defining an outer edge of the central strip 2155, defines an inner edge of a respective one of the first flank 2156 and the second flank 2158. As explained in more detail with reference to FIGS. 23A-23D, a slider for one or both of the zippers 2184 may be used to expand an overall longitudinal length of the belt 2150.

As illustrated in FIG. 21B, the exterior of the belt 2150 includes an exterior belt attachment segment 2172 provided on an exterior surface of the first flank 2156. In use, the patient 20 or the patient's caregiver may attach the central belt attachment segment 2162 to the first or second back attachment segment 240, 242, and wrap the first and second flanks 2156, 2158 around the patient's lumbar/waist region. An interior surface of the second flank 2158 may overlap an exterior surface of the first flank 2156 so that the interior belt attachment segment 2126 engages and attaches to the exterior belt attachment segment 2172. An exterior belt attachment segment 2172 is also provided on an exterior surface of the second flank 2158.

Belt grips 2180 may be provide on the exterior 2154 of the belt 2150. At least one belt grip 2180 may be attached to the surface of the belt exterior 2154 on each side of the central strip 2160 via respective grip attachments. Similar to grips provided on the vest 200, the grip attachments include grip ends attached to the surface of the exterior 2154 by grip stitching 2182 that may be visible on the belt interior surface (See FIG. 21A).

The belt 2150 includes vents 2186 positioned inwardly of the belt grips 2186 relative the central strip 2155. The vents 2186 are in a folded position in FIG. 21B as a result of the zippers 2184 illustrated in FIG. 21A being closed. As explained in more detail with reference to FIGS. 23A-D, when either of the zippers 2184 is opened such that opposing chains are disengaged, the vents 2186 can be extended laterally to the extent that each defines a respective amount of longitudinal slack, and extend the longitudinal length of the belt 2150. The vents 2186 can be formed from the same material a primary portion of the belt 2150 to which components such as the attachment segments 2126, 2158, 2172 and the grips 2180 are attached. The belt including its components can, in turn, be formed from the same materials as the vest 200 and corresponding components thereof.

A reinforcement segment 2188 covers, and may provide attachment points for, one or more elastic tension bands 2190. Each elastic tension band 2190 includes a band body 2192 and a band end 2194 extending from the band body 2192. Reinforcement-type stitching 2196 (hereafter referred to as “band stitching 2196”) similar to reinforcement stitching of the grip stitching 2182 may attach an attachment segment 2198 (See FIG. 23A, hereafter referred to as “band attachment segment 2198”) to the band end 2194.

In use, the band ends 2194 may be passed through spaces between respective belt grips 2180 and a surface of the exterior 2154, and the band attachment segments 2198 may be attached to respective exterior belt attachment segments 2172. If both elastic tension bands 2190 are not extended beyond respective normal state lengths prior to attachment to the exterior belt attachment segments 2172, the belt 2150 will remain in the passive state. A portion of the belt 2150 capable of recoiling is limited to portions of the central strip 2155 between outer edges of the central belt attachment segment 2162 and locations where the band ends 2194 are attached to respective third belt attachment segments 2110. Where the elastic tension bands 2190 are in a tensile state, and the belt 2150 is thus in an active state, ends of the elastic tension bands 2190 attached to the belt 2150 are in a state of tension. In a situation where the compression vest system 2100 is used by a patient that has extended the elastic tension bands 2190 to a maximum length, substantial tensile forces may be exerted on attachment points between the elastic tension bands 2190 and the belt 2150.

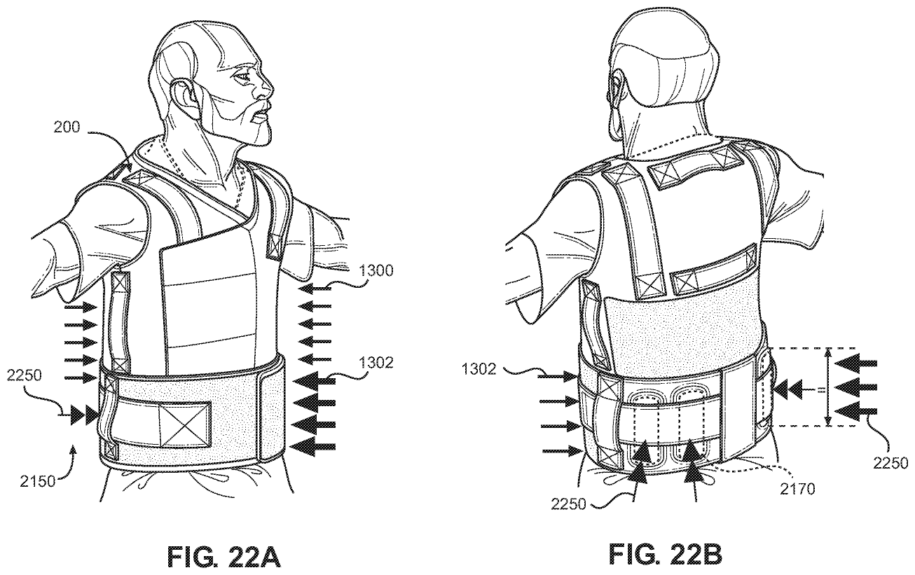

FIG. 21B further illustrates second pocket layers 2166B of the pockets 2164 of the segmented pouch 2160. As indicated by the undulations of the layers 2166 illustrated in FIGS. 21A and 21B, the stays 2170 can have thickness so as to cause the layers 2166 to bulge beyond a normal thickness of a primary portion of the belt 2150 to which grips and attachment segments are attached. In one example, a thickness, number, and configuration of the stays 2170 will be determined according to location and magnitude of loads on a sacral-lumbar area of a patient to be relieved. As explained in more detail with reference to FIGS. 22A and 22B, the combination of the vest 200 and the belt 2150, with the segmented pouching 2160, can provide a patient with targeted lower spinal support.

FIGS. 22A and 22B illustrate front and rear perspective views of the compression vest system 2100, according to an aspect of the present disclosure. In the configuration illustrated: (1) the first panel 210 is attached to the second panel 220 via the closure 300; (2) the belt 2150 is attached to the vest 200 via the central belt attachment segment 2162 and the first back attachment segment 240; and (3) the first and second flanks 2156, 2158 are wrapped around a torso of the patient 20 such that the first belt attachment segment 2156 (not shown) is attached to the exterior belt attachment segment 2172 provided on the first flank 2156. In addition, the elastic tension bands 2190 have been attached to the first and second flanks 2156, 2158 so as to be in a state of tension. The first compressive force 1300 applied by the vest 200 provides general support to a patient's spine, especially with respect to opposing movement of a patient's spine in sagittal and coronal body planes.

Adding the belt 2150 so as to be tightly or form-fittingly (with respect to a form of the patient 20) wrapped around the vest 200 worn by the patient 20 will result in second compressive forces 1302 generally/broadly exerted on the lower torso of the patient 20, particularly in a lumbar and sacral region. Further, the belt 2150, even without the segmented pouch 2160, generally assists in controlling movement of a patient's spine in the sagittal plane and supports the patient's lumbar/sacral region. Extending the elastic tension bands 2190 beyond normal state lengths and attaching them to the exterior belt attachment segments 2172 increases the second compressive forces 1302 exerted on the patient 20.

The first and second compressive forces 1300, 1302 counter act forces applied to the compression system 200 resulting from any of the first and second front, first and second back, body, and belt grips 218, 228, 236, 238, 280, 2120 being pulled or pushed in any direction. As a result, the first and second compressive forces 1300, 1302 act to: (A) support the spine in sagittal and coronal body planes; and (B) resist and eliminate, or at least substantially minimize a vertical displacement, a rotational displacement, and a front to back displacement of the vest 200 and the belt 2150 relative to the patient 20. Further, the addition of the segmented pouch 2160 enables the compression vest system 2100 to provide targeted load relief to vertebrae in a patient's spine.

Each stay 2170 in the segmented pouch 2160 can exert a compressive force over a respective portion of a lumbar and sacral spinal region of a patient. More specifically, with the elastic tension bands 2190 extending over the portion of the central strip 2155 including the segmented pouch 2160 and placed in tension, each stay 2170 in the segmented pouch 2160 applies an individual compressive force 2250 on a respective first pocket layer 2166A of the belt 2150, which is transmitted over a corresponding area of the back panel 230 of the vest 200. In the case of the belt 2150, the individual compressive forces 2250 are directed to a posterior of the patient in the lumbar and sacral portions of the patient's spine.

A cumulative effect of the individual compressive forces 2250 applied over a total surface area of a corresponding region of the vest 200, and thereby the patient, may produce intracavitary pressure that reduces load on intervertebral discs. As a result, a degree of reduction in load on the intervertebral discs in an area of the spine between the first (“L-1”) and just below the fifth vertebrae (“L-5”) of the lumbar spine, may be achieved. Such a reduction may be proportional to a cumulative magnitude of the individual compressive forces 2250 applied by the stays 2170 of the belt 2150. Accordingly, a patient utilizing the compression vest system 2100 may experience a substantial improvement in stability in their lumbar spine.

FIGS. 23A and 23B respectively illustrate elevation views of the exterior 2154 and interior 2152 of the belt 2150 in a compact state, whereas FIGS. 23C and 23D respectively illustrate elevation views of the same portions of the belt 2150 in an expanded state. As illustrated in FIG. 23A, vents 2186 are folded inwards toward the reinforcement segment 2188 with the zippers 2184 in a closed state. The vents 2186 may be sized to have a desired width in order to provided a desired degree of overall extension for the belt 2150. In one example, the vent 2186 may be sized to accommodate a piece of equipment that a patient may have to use or wear to some degree of regularity, but not all of the time. In another example, where the patient may be expected to grow a certain amount in a predictable amount of time, the vents 2186 may extend a certain amount so the patient can use their compression vest system 2100 until a next different size is required.

With the belt 2150 in the compact state, the vents 2186 may protrude from a surface of the exterior 2154 of the belt 2150. However, with the elastic tension bands 2190 extended through the spaces between the surface of the exterior 2154 and the belt grips 2180 and attached to the exterior belt attachment segments 2172, each elastic tension band 2190 can press down a respective vent 2186. As result, any bulge that may result from vents 2186 being folded will be minimized by the normal function of the elastic tension bands 2190. Accordingly, the vents 2186 will not obstruct an individual's ability to run their hand between one of the belt grips 2186 and the belt exterior 2154.

As illustrated in FIG. 23B, the zippers 2184 of the belt 2150 are closed. The sliders for each zipper 2184 are positioned at an upper edge of the belt 2150. As one of ordinary skill in the art would readily recognize, moving the slider towards the bottom edge of the belt 2150 will cause opposing chains to disengage until the slider is moved to a stopper of one chain that is beyond the last tooth of the opposing chain, thereby separating the edges once joined. FIGS. 23C and 23D illustrate a case in which both of the zippers 2184 have been opened. The vents 2186 are therefore shown in an extended state.

In particular, first and second vent edges 2186A, 2186B are shown. The first vent edge 2186A of each zipper 2184 may define or substantially correspond to an inner edge of an edge of a respective one of the first flank 2156 and the second flank 2158. Each of the second vent edges 2186B defines or substantially corresponds to a respective outer edge of the central strip 2155.

In one example, the vent 2186 includes first and second chains 2184A, 2184B of the zipper 2184 illustrated in FIG. 23D. Thus, the vent includes a section of fabric with long edges that either define or have attached thereto, tape on which a chain of teeth is included. Accordingly, either the tape or an edge of the section of fabric there attached is also attached to a respective outer edge of the central strip 2155 and an inner edge of a respective one of the first and second flanks 2156, 2158. In another example, the tape and chain are incorporated in the edges of the central strip 2155 and the first and second flanks 2156, 2158. In that case, the long edges of the section of fabric defining the vent 2186 will be sewn or otherwise permanently attached to an edge of the central strip 2155 and a respective one of the first and second flanks 2156, 2158.

In either configuration for the vent 2186 discussed above, the vent 2186 may include two layers. A first layer may be formed of elastic material and define a portion of the belt exterior 2154 in an expanded state of the belt 2150. The second layer may be made of inelastic material and define a portion of the belt interior 2152 in the expanded state. As a result, the elastic layer in the compact state will limit a degree that the in-elastic layer will bulge out, and the in-elastic layer can limit an extent that the elastic layer can be stretched in the expanded state of the belt 2150.

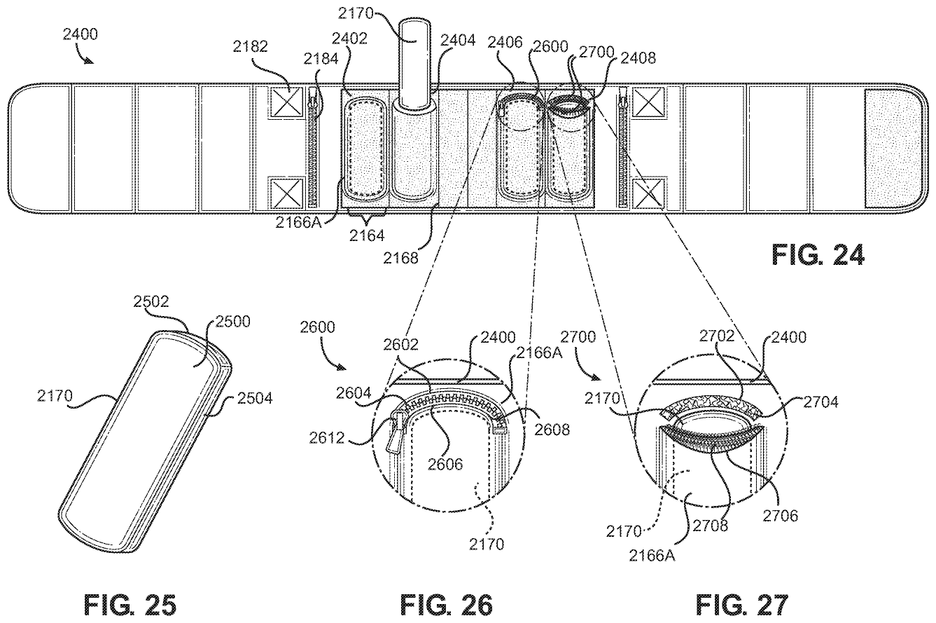

FIG. 24 illustrates an elevation view of a belt 2400 including removable stays 2170, according to an aspect of the present disclosure. In particular the segmented pouch of the belt 2400 includes first, second, third, and fourth versions 2402, 2404, 2406, 2408 of the pocket 2164. The first pocket 2402 is closed like the pockets 2164 provided in the belt 2100 illustrated in FIGS. 21-23D. The second pocket 2404 is a version of the first pocket 2402 that has be cut adjacent to a top edge of the belt 2400, or otherwise opened by making a hole in the first pocket layer 2166A, to remove a respective stay 2170. Thus, the first pocket 2402 is configured to permanently retain a stay 2170 unless altered in such a way that a repair would be necessary. A close up of a stay 2170 that may correspond to the stay 2170 removed from the second pocket 2404 is illustrated in FIG. 25.

The stay 2170 illustrated in FIG. 25 may be a rigid panel having front and back surfaces 2500 bordered by short edges 2502 and long edges 2504. As illustrated, the stay 2170 is provided in a shape of a rectangular box having rounded corners. The surfaces 2500 may be flat or contoured so as to be convex or concave relative to a plane extending along a longitudinal axis of the stay 2170. The surfaces 2500 of stay 2170 may be formed with a degree of curvature suited for a location of a patient's body that the particular stay 2170 is configured to support. Accordingly, the stay 2170 in the first pocket 2402 may have a different shape or surface contour as compared to the stays in the second, third, or fourth pockets 2404, 2406, 2408. In yet another example, the faces of the short and long edges 2502, 2504 of the stay 2170 extending parallel to a plane extending along a longitudinal axis of the stay 2170, may be substantially flat, and one or both of the surfaces may define concave or convex profile along the longitudinal length of the stay 2170. In still another example not shown, the stay 2170 may be provided in the shape of the rectangular box having rounded corners as with the stay of FIG. 25, but further include beveled edges for the short and/or long edges 2502, 2504.

As applied to the stay 2170 illustrated in FIG. 5, the above discussion generally defining a stay according to the present disclosure provides that that the stay 2170 may be a rigid or semi-rigid type of panel, layer, board, or slat. Further, the stay 2170 illustrated in FIG. 25 may be provided by a flat piece of hard material such as a lightweight metal (e.g., titanium, aluminum, etc.), or a thermoplastic material such as a polycarbonate (e.g, Lexan). In other examples, the stay 2170 may be provided by a strip of semi-flexible plastic having a degree of elasticity suitable for the spinal support needs of a particular patient. As with the shape of the stays 2170 for the different pockets of the belt 2400, materials for the stay 2170 in the first pocket 2402 may differ from materials used for the stays 2170 in the second, third, and fourth pockets 2404, 2406, 2408 in order to provide a desired level of load reduction in a desired location on a patient.

The third and fourth pockets 2406, 2408 provide alternative configurations that allow respective stays 2170 to be removed, reinserted, or replaced with different sized stays as needed by a patient. In particular, the third pocket 2400 includes a zippered access 2600, and the fourth pocket 2408 provides hook and loop access 2700.

FIG. 26 illustrates a close up of the zippered access 2600 of the third pocket 2406. As illustrated, a first tape 2602 may be attached to the first pocket layer 2166A in an area between a top edge of the belt 2400 and a top of an opening in the first pocket layer 2166A. The first tape 2602 includes a first chain 2604 of teeth. A second tape 2606 including a second chain 2608 of teeth may be attached to a bottom of the opening in the first pocket layer 2166A, which may be correspond to an area of the first pocket layer 2166A disposed just above a top short edge 2502 of a stay 2170 when the stay 2170 is positioned in the third pocket 2406. A slider 2612 may be moved from a left side to a right side to open the opening in the first pocket layer 2166A so that the stay 2170 can be removed from or inserted in to the third pocket 2406.

FIG. 27 illustrates a close up of the hook and loop access 2700 of the fourth pocket 2408. As illustrated, a first strip 2702 including a first attachment segment 2704 of hook or loop material is sewn, glued, or otherwise attached to the first pocket layer 2166A in an area between the top edge of the belt 2400 and a top of an opening in the first pocket layer 2166A. A second strip 2706 including a second attachment segment 2708 of loop or hook material may be attached to a bottom of the opening in the first pocket layer 2166A, which may be correspond to an area of the first pocket layer 2166A disposed just above a top short edge 2502 of a stay 2170 when the stay 2170 is positioned in the fourth pocket 2408. The second strip 2706 may be pulled apart from the first strip 2702A to open the opening and either remove the stay 2170 or place it in the fourth pocket 2408. As will be understood the second strip 2706 may be pressed into locked engagement with the first strip 2706 to close the opening and securely hold the stay 2170 within the fourth pocket 2408.

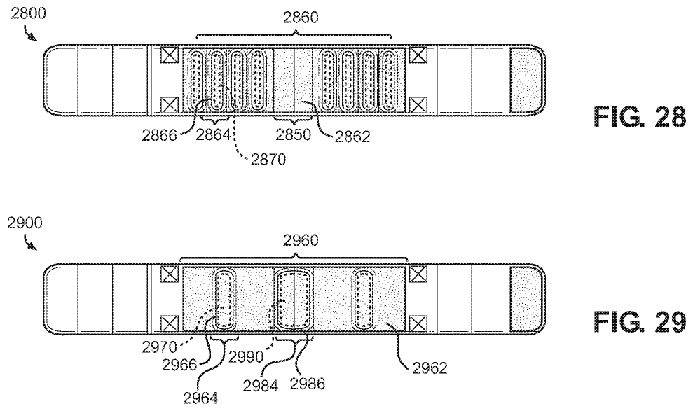

For the purposes of this disclosure, the size and shape of the pockets 2164 and stays 2170 for the belts 2150, 2400 illustrated in FIGS. 21A-28 may be considered a standard size and shape. Thus, these components may be referred to hereafter as the “standard pocket 2164” and the “standard stay 2170.” As noted above, different sizes, shapes, and configurations of stays may be provided in a segmented pouch to optimize a magnitude, orientation, and location of a desired load relief on a patient's spine. As such, FIGS. 28-41A illustrate different configurations of belts for a compression system according to the present disclosure.

FIG. 28 illustrates an elevation view of a belt 2800 including a segmented pouch 2860 with a series of stays 2870 having a narrow width (“narrow stay 2870”). Pockets 2864 defined by vertical stitching in pocket layers 2866 on an interior and exterior of the belt 2800 have a width to accommodate to a size of the narrow stays 2870 (“narrow pockets 2864”). The pocket layer 2866 illustrated in FIG. 28 may span an entire length of and define a first attachment layer 2862 of the belt 2800. The narrow pockets 2864 may be divided into groups of four narrow pockets 2864 on opposite sides of a central region 2850 of the belt 2800.

FIG. 29 illustrates an elevation view of a 2900 belt including a segmented pouch 2960 including first and second intermediately sized stays 2970, 2990, according to an aspect of the present disclosure. In particular, a first attachment segment 2962 defines a pocket layer 2966 is segmented into first and second intermediate sized pocket layers 2966, 2968 by vertical stitching to provide first and second intermediate sized pockets 2964, 2984. The first intermediate sized pockets and stays 2964, 2970, are slightly wider than the narrow pockets and stays 2864, 2870, and wider than the standard pockets and stays 2164, 2170. Further the first intermediate sized pockets and stays 2964, 2970 are positioned equidistantly to the left and right of a center of the belt 2900. The second intermediate sized pocket 2984 is wider than the standard pocket 2164, and may be provided at the center of the belt 2900.

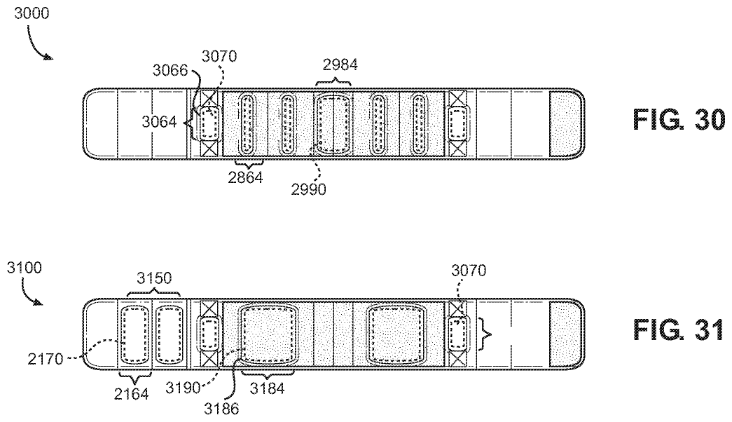

FIG. 30 illustrates an elevation view of a belt 3000 including narrow, second intermediate sized, and short stays 2870, 2990, 3070, according to an aspect of the present disclosure. The narrow stays 2870 are provided in narrow pockets 2870 position equidistantly to the left and right of a center of the belt 3000. The short stays 3070 are positioned in short pockets 3064 formed by pocket layers 3066 provided vertically between stitching for belt grips of the belt 3000. In this location the short stays 3070 can apply compressive forces from opposing lateral-most sagittal planes relative to a midline of a patient's trunk when elastic tension bands are positioned in tension.

FIG. 31 illustrates an elevation view of a belt 3100 including wide and short stays 3190, 3070, according to an aspect of the present disclosure. The wide stays 3190 are positioned within wide pockets 3184 defined by vertical stitching in pocket layers 3186 of the belt. Previous examples of segmented pouches according to the present have been located in portions of respective belts that in use, correspond to a posterior of a person wearing the belt. The belt 3100 illustrated in FIG. 31 includes an anterior segmented pouch 3150 including standard stays 2170. When the belt 3100 is positioned on a patient, localized individual compressive forces will be applied to anterior side of an individual's lumbar and sacral region.

In general, the anteriorly positioned stays 2170 may provide the additional support required to those individuals who experience mild to severe decreases in trunk control. The anteriorly positioned stays 2170 may also provide structural support that yields overall functional improvements in those individuals with mild to severe alignment and structural spine issues like scoliosis. In addition, the anteriorly positioned stays 2170 can provide protection and an important barrier to prevent injury or pain after surgeries like open heart surgeries. For example, stiches or staples employed to close an incision of a patient who has undergone open heart surgery, will be restricted from moving apart and shielded from external objects by the stays 2170. Finally, the anteriorly positioned stays 2170 can provide an individual wearing the compression vest system 2100 according to the present disclosure with much needed protection as compared to various “turtle shell” solutions. Said individual may experience increased comfort, movement, function, and more importantly quality of life post-surgery by wearing the compression vest system 2100 including a belt with anteriorly positioned stays.

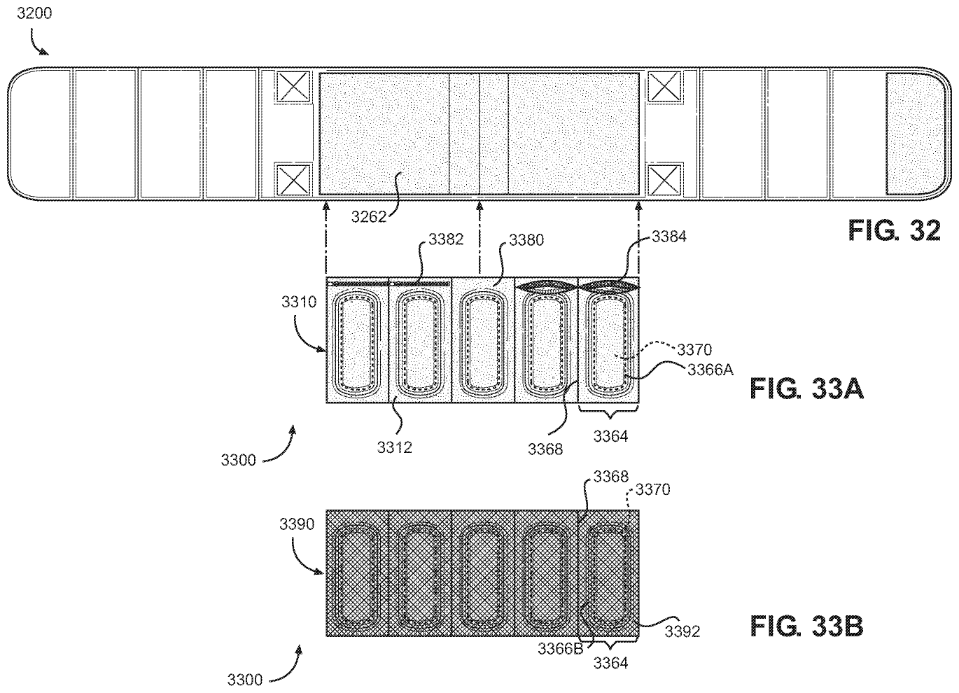

FIG. 32 illustrates an elevation view of a belt 3200 that includes a first modular segmented pouch 3300 (“first modular pouch 3300”) illustrated in FIGS. 33A and 33B. The belt 3200 includes a central attachment segment 3262 that is configured to attach to a first pouch attachment segment 3312 on an exterior 3310 of the first modular pouch 3300. The first modular segmented pouch 3300 includes a plurality of pockets 3364 defined by vertical stitching 3368 attaching first and second pocket layers 3366A, 3366B. Each pocket 3364 may include a stay 3370 that may be identical to the standard stay 2170 illustrated in FIG. 25. The first pocket layer 3366A for each pocket 3364 may have a closed configuration 3380, a zippered opening 3382, or a hook and loop access 3384. The stays 3370 for the pockets 3364 including either the zippered opening 3382 or the hook and loop access 3384 can be removably placed in respective pockets 3364.

The second pocket layer 3366B defines an interior 3390 of the modular segmented pouch 3300, and can be formed with a cushioned layer 3392 of material. In one example the cushioned layer 3392 may be formed from neoprene. Further, the second pocket layer 3366B can be provided with a surface that also provides an attachment segment. The attachment segments of the first modular pouch 3300 are configured to attach to the central belt attachment segment 3262, and the first or second attachment segments 240, 242 of the vest 200.

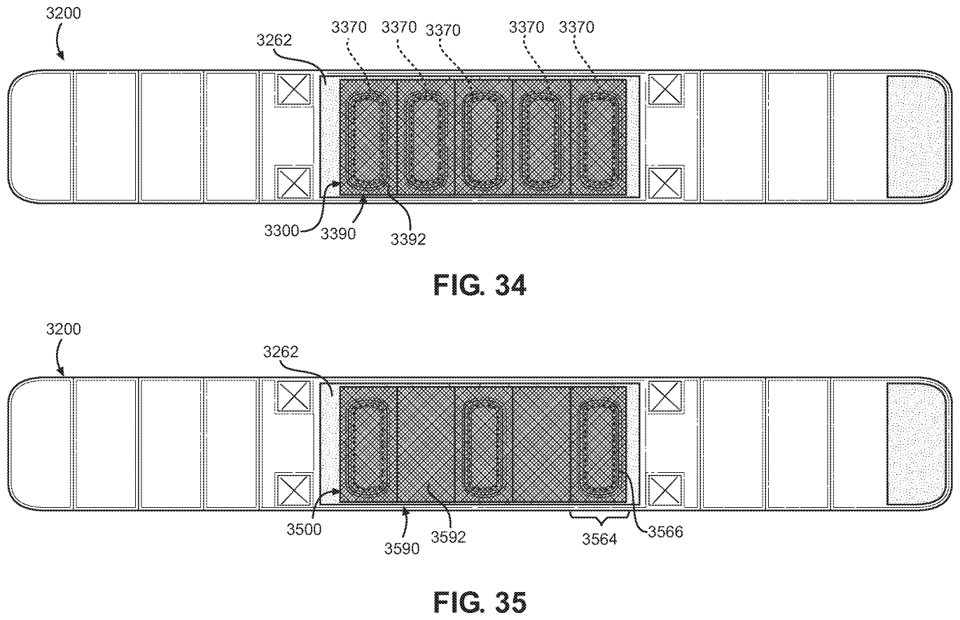

FIG. 34 illustrates an elevation view of the belt 3220 with the first modular pouch 3300 attached. More specifically, the first pouch attachment segment 3312 provided by the first pocket layer 3366A is attached to the central belt attachment segment 3262. Accordingly, the cushioned neoprene second pocket layer 3366B will be pressed against a patient's back and provided cushioned support that may increase overall comfort.

FIG. 35 illustrates an elevation view of the belt 3200 with a modified first modular segmented pouch 3500 (“modified first modular pouch 3500”) attached. The modified first modular pouch 3500 may include a different configuration of pockets 3564 defined by vertical stitching attaching pocket layers 3564 as compared to the first modular pouch 3300 illustrated in FIGS. 33A, 33B, and 34. However, like the first modular pouch 3300, the pocket layer 3566 defining an interior 3590 of the modified first modular pouch 3500 may include a cushioned layer 3592 of material.

In another example, the modified first modular pouch 3500 is the same pouch as the first modular pouch 3300. As illustrated in FIG. 33A, the first modular pouch 3300 includes removable stays 3370. Accordingly, the modified first modular pouch 3500 may be the first modular pouch 3300 with the stays 3370 removed from the pockets 3364 on opposite sides of the central pocket 3364 illustrated in FIG. 33A that includes the closed configuration 3380.

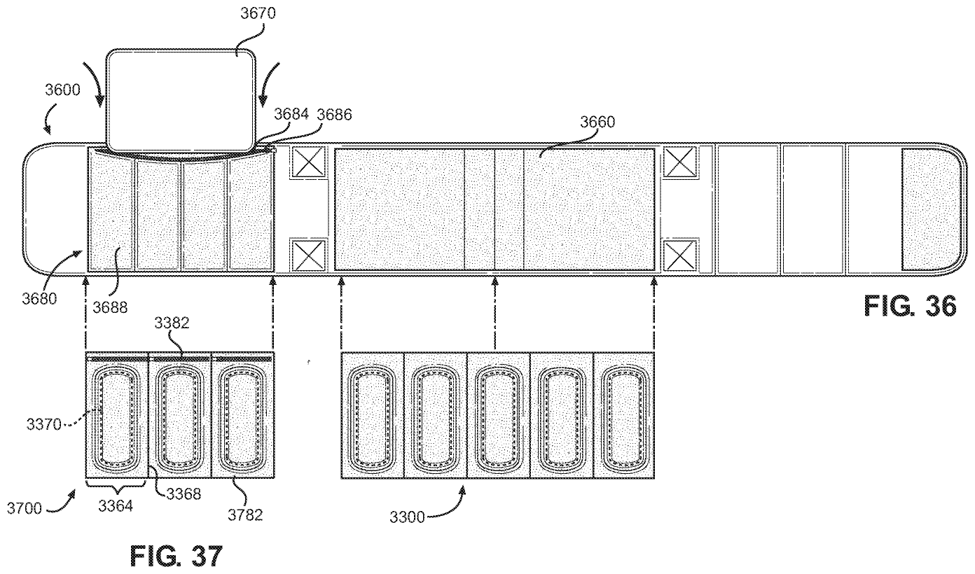

FIG. 36 illustrates an elevation view of a belt 3600 that includes a central attachment segment 3660 sized and configured to attach to the first modular pouch 3300. The first modular pouch 3300 will be positioned to apply targeted compressive forces to a posterior of a patient. In addition, the belt 3600 includes an anterior pocket 3680 configured to receive either or both of an extra-wide stay 3670 and a second modular segmented pouch 3700 (“second modular pouch 3700) illustrated in FIG. 37.

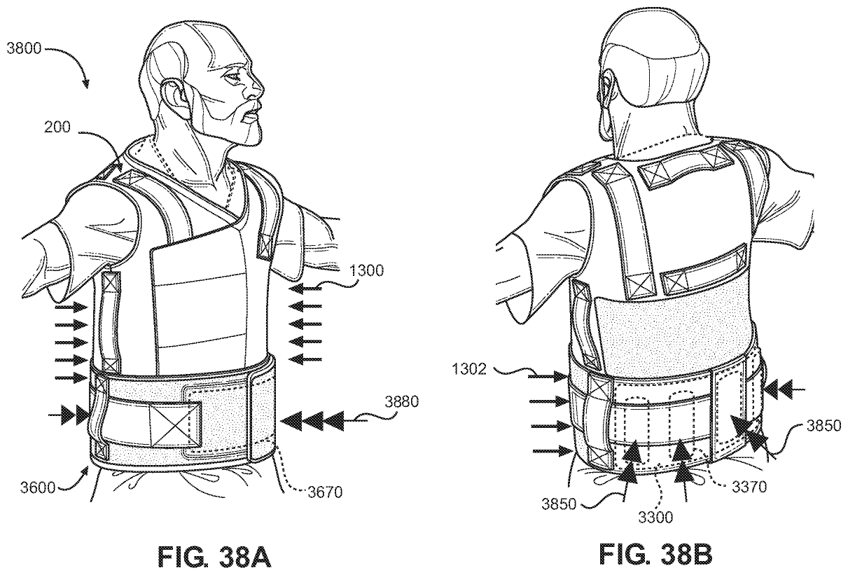

The extra-wide stay 3670 may be received or removed through an opening 3684 in the anterior pocket 3680 that may include opposing fastening strips 3686 with zipper chains or strips of hook and loop material. In practice: the opening 3684 may be opened by moving a slider of a zipper or pulling one fastening strip 3686 away from the other where a hook and loop access is utilized; the extra-wide stay 3670 can be positioned in the pocket 3680; and the opening 3684 can be closed. As explained with reference to FIGS. 38A and 38B, the single wide shape of the extra-wide stay 3670 may be particularly beneficial to patients that have recently undergone open heart surgery, who need assistance with trunk control while at the same time needing to protect staples or stitches where an incision was made. A width of the extra-wide stay 3670 (i.e., a dimension extending along a longitudinal length of the belt 3600) may be substantially equal to a width of an abdomen area of a patient wearing a compression vest system according to the present disclosure. The same options for material, edge-shape, and surface contour that can be used to provide the stays 2170, 2870, 2970, 3070, 3170 may be employed to provide the extra-wide stay 3670. Further, the extra-wide stay 3670 can be formed with edges, surface contours, and an overall curvature that is customized to the shape of patient's abdomen so as to promote comfort.

An exterior surface 3688 of the anterior pocket 3680 illustrated in FIG. 36 defines an attachment segment (“anterior attachment segment 3688″) configured to attach to a pouch attachment segment 3782 of the second modular pouch 3700. In one example, the anterior attachment segment 3688 extends over an entire width of the anterior pocket 3680, which substantially corresponds to a width of the of the extra-wide stay 3670. This in turn, may provide an attachment segment having a width the same as second modular pouch 3700. In another example, the extra-wide stay 3670 having a smaller width may be provided, and the anterior pocket 3680 may be configured with vertical stitching inward of the edges of the anterior attachment segment 3688 so as to define an opening that corresponds to the width of the extra-wide stay 3670.

The second modular pouch 3700 may include a plurality of pockets 3364 which are defined by stitching 3368 in the pouch attachment segment 3782 to have a same configuration (e.g., size) as the pockets 3364 of the first modular pouch 3300. However, as the second modular pouch 3700 is configured to attach to the attachment segment 3688 which may be smaller than the central attachment segment 3660 of the belt 3600, and the second modular pouch 3700 may have fewer pockets 3364 than the first modular pouch 3300 as illustrated. Each of the pockets 3364 may be provided with a zipper 3382 (or hook and loop access), so that respective stays 3370 may be removed or replaced.

It will be noted that both the first and second modular pouches 3300, 3700 may be configured with some or all of their respective pockets 3364 provided with zippered or hook and loop access openings so that each pouch may be variably configured to apply compressive forces to a patient's trunk where most effective. For example, the second modular pouch 3700 may have three pockets 3364 but only a middle pocket 3364 may include a stay 3370. Other configurations may provide stays 3370 only in the first and third pockets 3364. A configuration of stays in the second modular pouch 3700 can be coordinated with a configuration of stays 3370 in the first modular pouch 3300 so that the localized/individual/targeted compressive forces are applied equidistantly around a circumference defined by a cross-section of a patient's (radially compressed—given the action of the vest compression system 2100 including the belt 3600) trunk. One of ordinary skill in the art will recognize that different configurations of stays 3370 can be implemented via the first and second modular pouches 3300, 3700.

FIGS. 38A and 38B illustrate front and rear perspective views of a compression vest system 3800 including the vest 200 and the belt 3600. The belt 3600 is provided with the first modular pouch 3300 attached to the central belt attachment segment 3660 (not shown), and the extra-wide stay 3670 positioned in the anterior pocket 3680. As illustrated, the first modular pouch 3300 is provided with a stay 3370 in each of its respective pockets 3364.

As discussed above with reference to FIGS. 22A and 22B, the first compressive forces 1300 applied by the vest 200 provide general support to a patient's spine, especially with respect to opposing movement of the spine in sagittal and coronal body planes. Were the belt 3600, absent the extra-wide stay 3670 and the first modular pouch 3300, added to the vest 200 so as to be tightly or form-fittingly wrapped around the vest 200 worn by the patient 20, it still would cause the second compressive forces 1302 to be generally/broadly exerted on the lower torso of the patient 20. These second compressive forces 1302 being applied in the lumbar/sacral region of a patient have the effect of locking a trunk and hips of the patient relative to each other and a transverse body plane. With the trunk and hips locked in place in proper (or at least improved) spatial alignment relative to the transvers plane, movement and motor control of the muscles in the hips and lower back occur in a natural (or least less inhibited) manner for an individual for which movements involving these muscles are problematic.

However, the illustrated belt 3600 includes the extra-wide stay 3670 and the first modular pouch 3300 as noted above. Each stay 3370 in the first modular pouch 3300 can exert a respective individual compressive force over a respective portion of a lumbar and sacral spinal region of a patient. More specifically, each stay 3370 may apply an individual compressive force 3850 that is transmitted over a corresponding area of the back panel 230 of the vest 200. The first individual compressive forces 3850 are directed to a posterior of the patient in the lumbar and sacral portions of the patient's spine and may produce intracavitary pressure that reduces load on intervertebral discs. Further, the individual compressive forces 3850 may combine with the second compressive forces 1302 to increase a vector of force that locks the trunk and hips of the patient relative to each other and the transverse body plane. Accordingly, the stays 3370 can enhance an ability of the compression vest system to maintain the patient's trunk and hips in spatial alignment.

Turning to the extra-wide stay 3670, the belt 3600 in FIGS. 38A and 38B is orientated on the patient with the extra-wide stay 3670 positioned to apply a second individual compressive force 3880 to an anterior of the patient. With the width of the extra-wide stay 3670 being substantially greater than that of other stays discussed herein, when the belt 3600 is placed on the vest 200, and respective tension bands are put in tension, the extra-wide stay 3670 exerts the second individual compressive force 3880 over an area substantially greater than the stays provided in the first modular pouch 3300, for example. More specifically, the second individual compressive force 3880 may be applied uniformly over an area of the vest 200, and thus over an area of an anterior of the patient, corresponding to at least the surface area of the extra-wide stay 3670.

As with the first individual compressive forces 3850, the second individual compressive force 3880 can combine with the other compressive forces 1300, 1302, 3850 to better limit spinal displacement in the sagittal and coronal body planes, provide support that facilitates improved trunk control for a patient, and lock the hips and trunk relative to each other and the transverse body plane. As discussed above, directing the compressive force to an anterior of an individual may provide the additional support required to those individuals who experience mild to severely decreased trunk control, and provide structural support that yields overall functional improvements in those individuals with mild to severe alignment and structural spine issues like scoliosis.

An added advantage of the size and shape of the extra-wide stay 3670 is how it can protect scares, wounds, incisions, or conceal and/or stabilize of devices, such as a catheter securement device. Each of the modular pouches described herein may offer some protection to a patient's incision, but the wide surface of the extra-wide stay 3670 ensures that only a substantially flat or ergonomically curved uniform plane of pressure is applied to the patient's skin where an incision or device may be disposed. Accordingly, pulling of the skin in different directions that may test an ability of sutures or staples to keep the incision closed may be minimized. Further, staples or devices like catheters are less likely to be caught on or moved by undulations in an individual's clothes, in particular when the individual moves, with the extra-wide stay 3670 applying the second individual compressive force over at least a respective surface area.

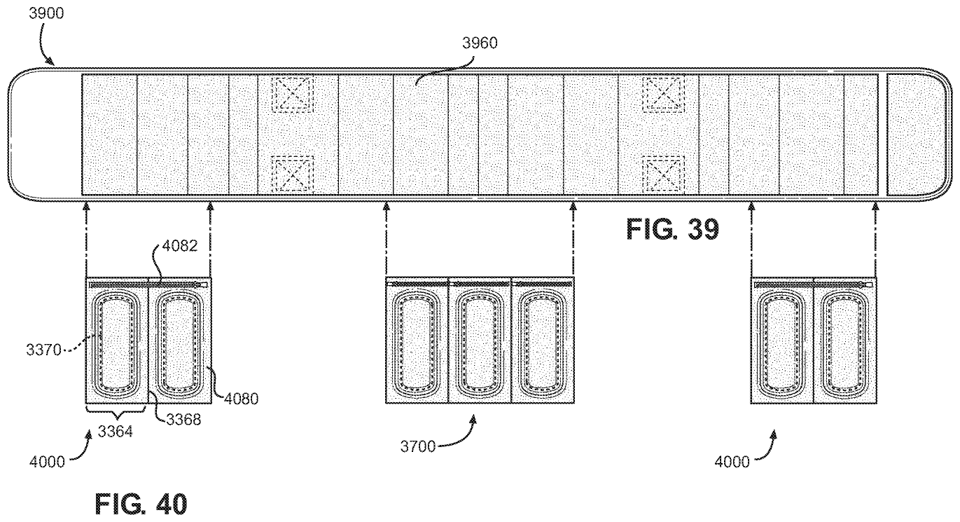

FIG. 39 illustrates an elevation view of a belt 3900 that includes an extended central attachment segment 3960 sized and configured to attach to multiple modular segmented pouches such as the second modular pouch 3700, and one or more third modular segmented pouches 4000 (“third modular pouch 4000) illustrated in FIG. 40. As shown, the second modular pouch 3700 can be positioned on the extended central attachment segment 3960 to apply targeted compressive forces to a posterior of a patient. The third modular pouches 4000 can be positioned to apply targeted compressive forces to respective anterior areas of the patient.

The third modular pouch 4000 may include two pockets 3364 which are defined by stitching 3368 incorporated in a pouch attachment segment 4080 so as to have a same configuration (e.g., size) as the pockets 3364 of the first modular pouch 3300. As only two pockets 3364 may be provided in the third modular pouch 4000, one single zipper 4082 (or hook and loop access) that can open and close both pockets 3364 may be employed. One of ordinary skill in the art will recognize this zipper or access configuration may be applied to any of the modular or fixed/integrated segmented pouches of the present disclosure. Conversely, the third modular pouch 4000 may include individual zippered or hook and loop accessible openings in each pocket 3364, that which can be opened and closed to access, remove, or a stay 3370 in a respective pocket 3364.

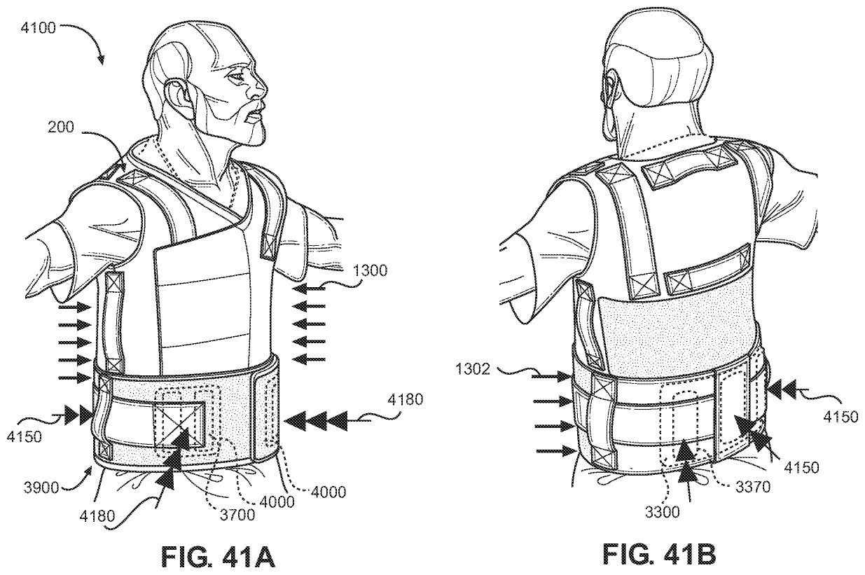

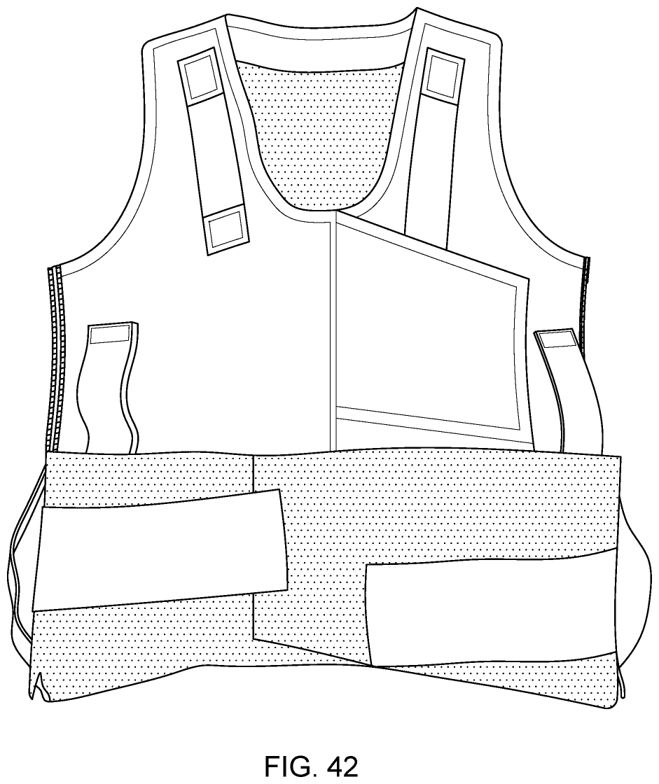

FIGS. 41A and 41B illustrates front and rear perspective views of a full implementation of a compression vest system 4100 including the belt 3900 with the second and third modular pouches 3700, 4000.

As discussed above with reference to FIGS. 22A, 22B, 38A, and 38B first compressive forces 1300 applied by the vest 200 provide general support to a patient's spine, especially with respect to opposing movement of the spine in sagittal and coronal body planes. Were the belt 3900 added absent the second and third modular pouches 3700, 4000, it still would cause the second compressive forces 1302 to be generally/broadly exerted on the lower torso of the patient, which can help lock a trunk and hips relative to each other and a transverse body plane.

Each stay 3370 in each of the second and third modular pouches 3700, 4000 can exert a respective compressive force over a respective portion of a lumbar and sacral spinal region of a patient. More specifically, each stay 3370 in the second modular pouch 3700 may apply a first individual compressive force 4050, and each stay 3370 in the third modular pouch may apply a second individual compressive force 4080 that is transmitted over a corresponding area of the back, first, and second panels 230, 210, 220 of the vest 200. The first individual compressive forces 4050 are directed to a posterior, whereas the second individual compressive forces 4080 are directed to an anterior of the patient of FIGS. 41A and 41B.

The third modular pouches 4000 are positioned substantially equidistant to a midline of the patient. Such positioning may be incorporated in an effort to directionally (vector direction) balance the forces being applied to the patient's trunk. However, the belt 4000 is advantageously configured to allow the multiple arrangements, each capable of applying a unique combination of directional forces on the patient. More specifically, the extended central attachment segment 3960 of the belt 3900 illustrated in FIG. 39, enables an individual, caregiver, or medical professional, to place modular segmented pouches in a variety of locations on the belt. When the belt 3900 is worn, each potential location for a modular pouch corresponds to a position along a circumference defined by an outer radial extent of a region of a patient including portions of their abdomen and lower back adjacent to a respective waist line. Thus, a caregiver, medical professional, or an individual prior to wearing a compression vest system, can customize where compressive forces are applied along the circumference. Thus, a cumulative effect of the all the compressive forces applied to a patient who is in moderate or significant need of trunk control support and assistance, can be tailored and optimized to obtain the most beneficial load reduction on the patient's spine, and yield the most significant functional improvement in the patient's muscles and joints during movement.

FIG. 42 illustrates a front view of a compression vest system according to an aspect of the present disclosure, wherein the compression vest system is in a closed position.

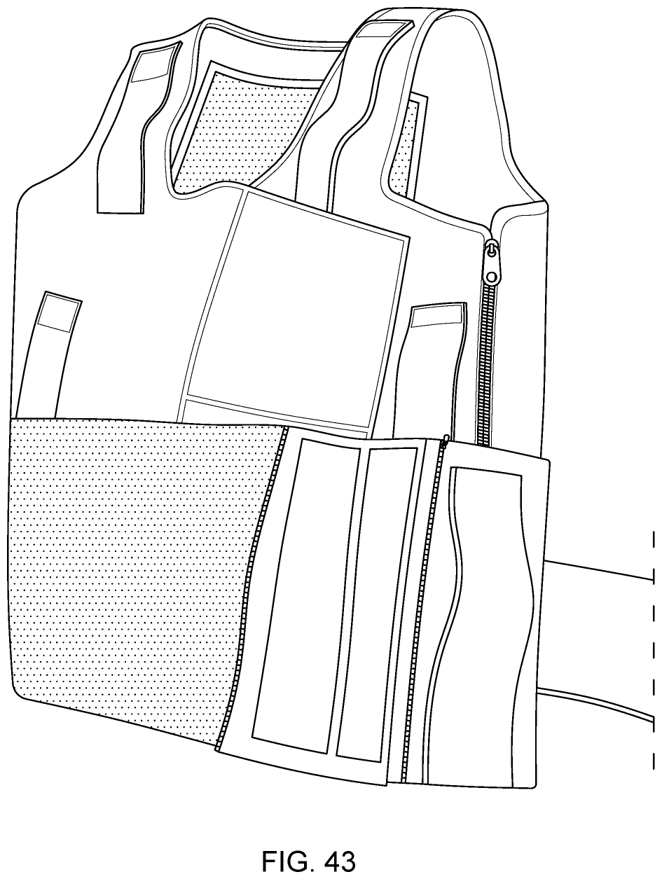

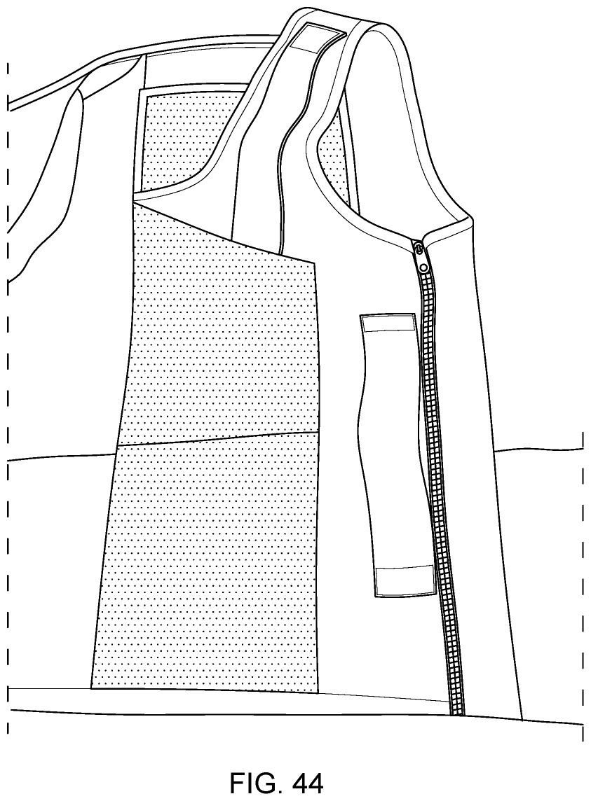

FIG. 43 illustrates a front, left side, perspective view of the compression vest system of FIG. 42, wherein the zipper expansion and retraction mechanism of the vest is in a closed position while the zipper expansion and retraction mechanism of the belt is in an open position.

FIG. 44 illustrates a front, left side, perspective view of the compression vest system of FIG. 42, wherein the vest is in a partially open position while the zipper expansion and retraction mechanism of the vest is in a closed position.

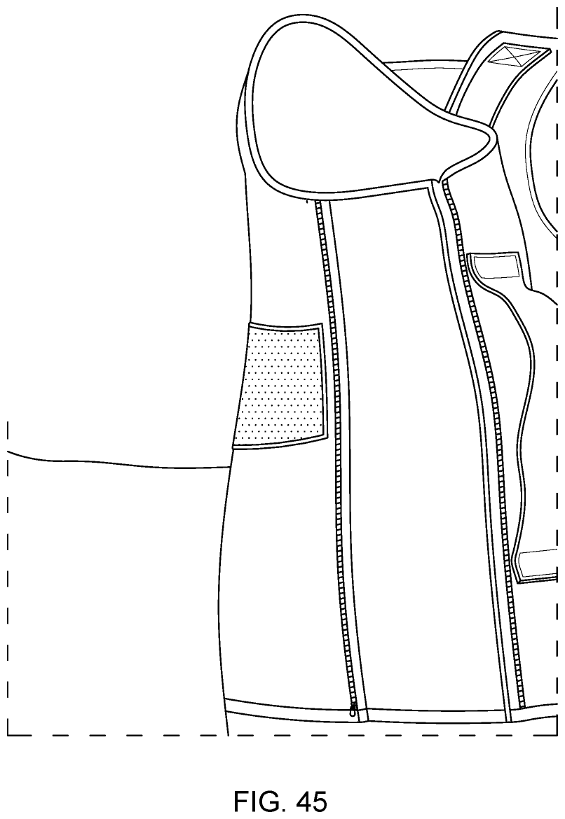

FIG. 45 illustrates a right side view of the compression vest system of FIG. 42, wherein the belt is in an open position while the zipper expansion and retraction mechanism of the vest is in an open position.

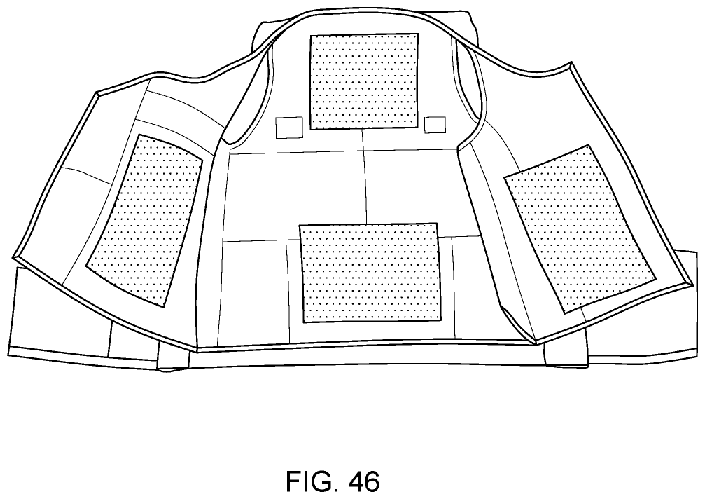

FIG. 46 illustrates a front view of the compression vest system of FIG. 42, wherein the compression vest system is in an open position.

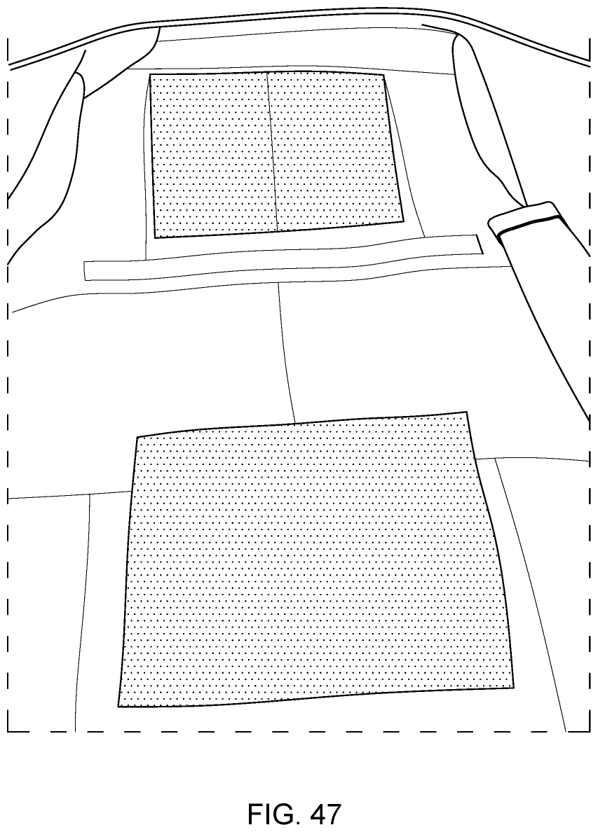

FIG. 47 illustrates a front view of the vest of the compression vest system of FIG. 42, wherein the vest is in the open position.

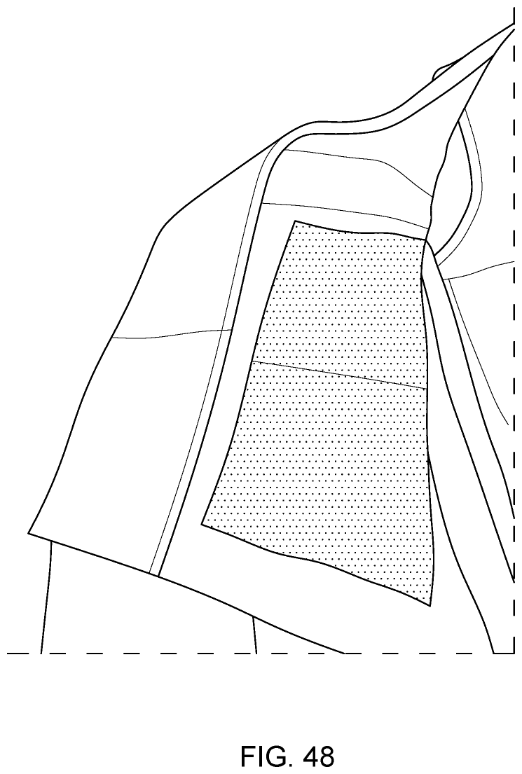

FIG. 48 illustrates a front, right side view of the vest of the compression vest system of FIG. 42, wherein the right side of the vest is in the open position.

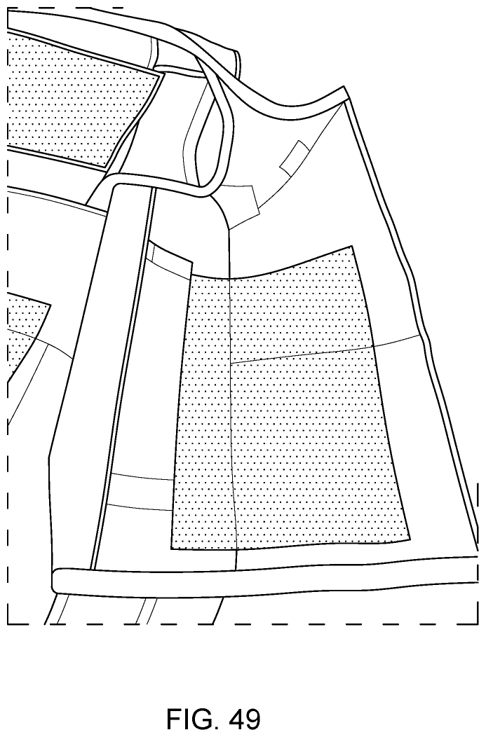

FIG. 49 illustrates a front, left side view of the vest of the compression vest system of FIG. 42, wherein the left side of the vest is in the open position.

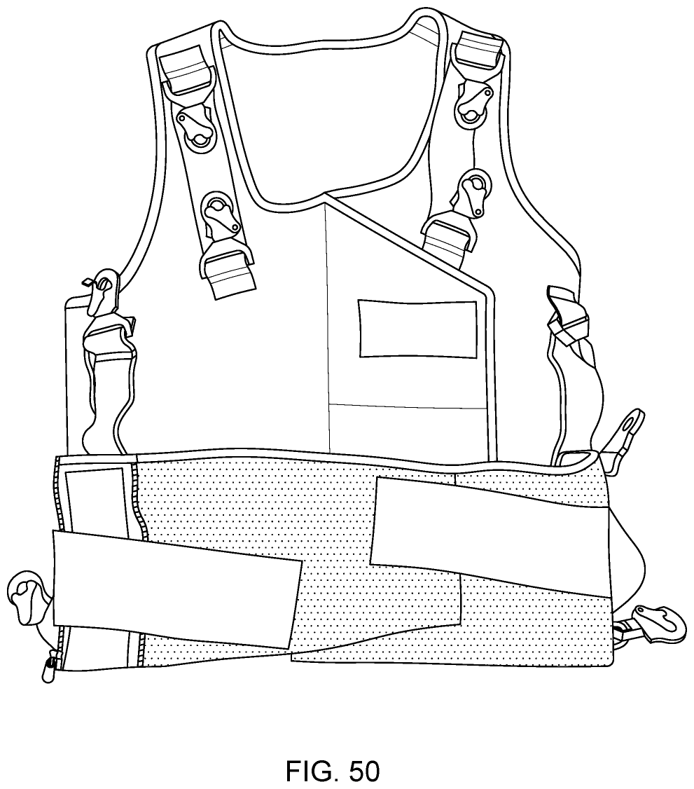

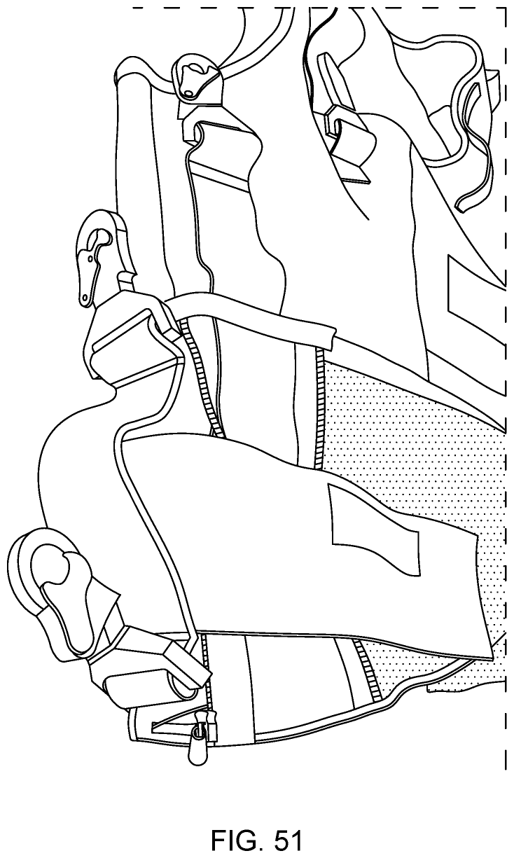

FIG. 50 illustrates a front view of a compression vest system according to an aspect of the present disclosure, wherein the compression vest system is in a closed position.

FIG. 51 illustrates a front, right side, perspective view of the compression vest system of FIG. 50, wherein the zipper expansion and retraction mechanism of the vest is in an open position and the zipper expansion and retraction mechanism of the belt is in an open position.

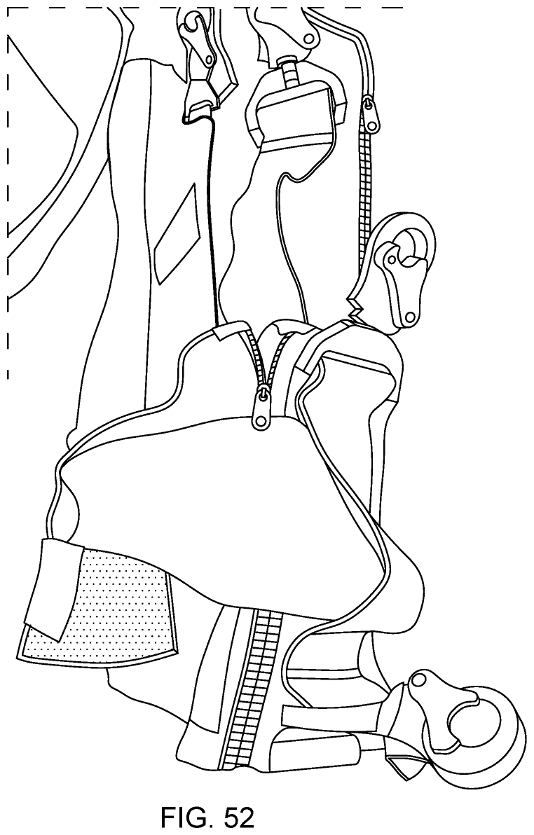

FIG. 52 illustrates a front, left side, perspective view of the compression vest system of FIG. 50, wherein the zipper expansion and retraction mechanism of the vest is in a closed position and the zipper expansion and retraction mechanism of the belt is in a closed position.

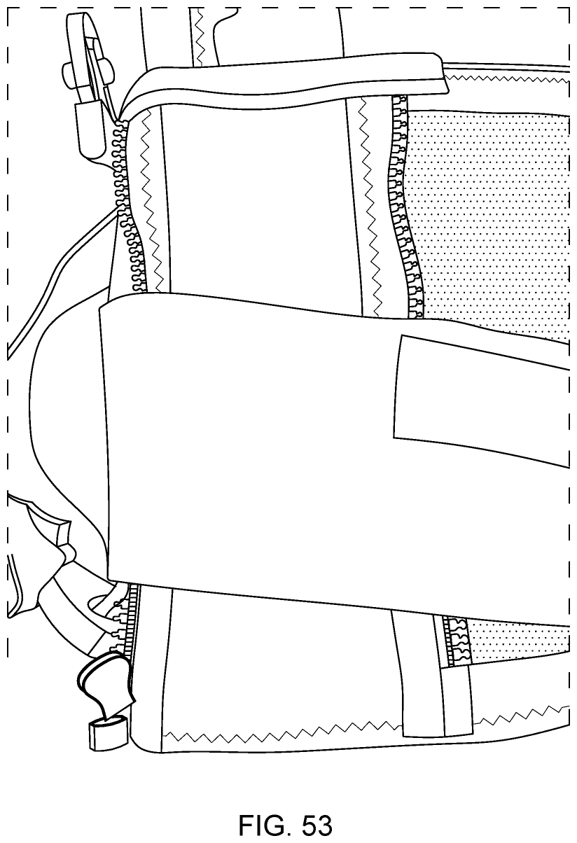

FIG. 53 illustrates a front, right side, perspective view of the belt of the compression vest system of FIG. 50, wherein the zipper expansion and retraction mechanism of the belt is in an open position.

FIG. 53 illustrates a front, right side, perspective view of the belt of the compression vest system of FIG. 50, wherein the zipper expansion and retraction mechanism of the belt is in an open position.

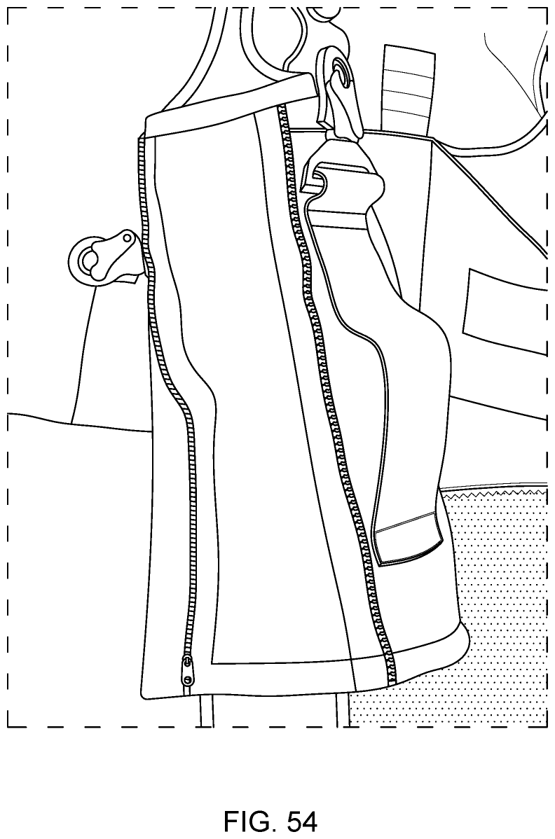

FIG. 54 illustrates a front, right side, perspective view of the vest of the compression vest system of FIG. 50, wherein the zipper expansion and retraction mechanism of the vest is in an open position.

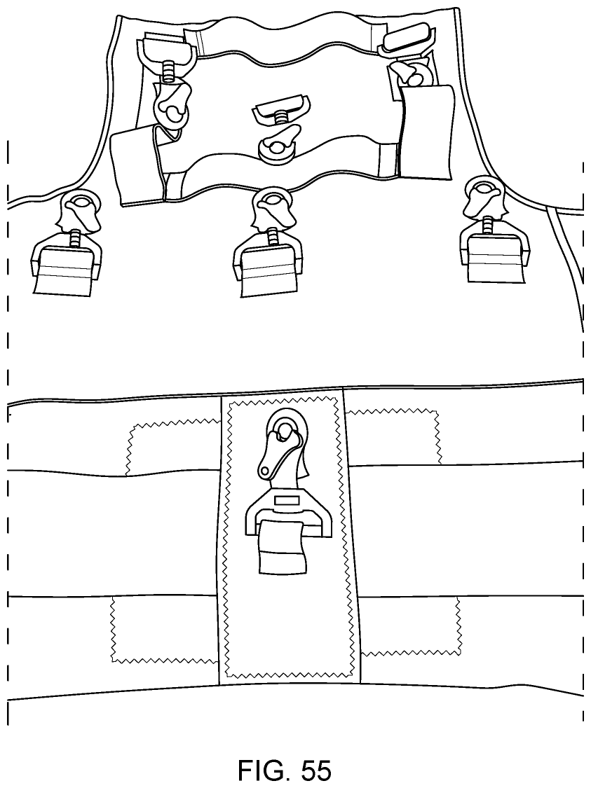

FIG. 55 illustrates a rear view of the compression vest system of FIG. 50.

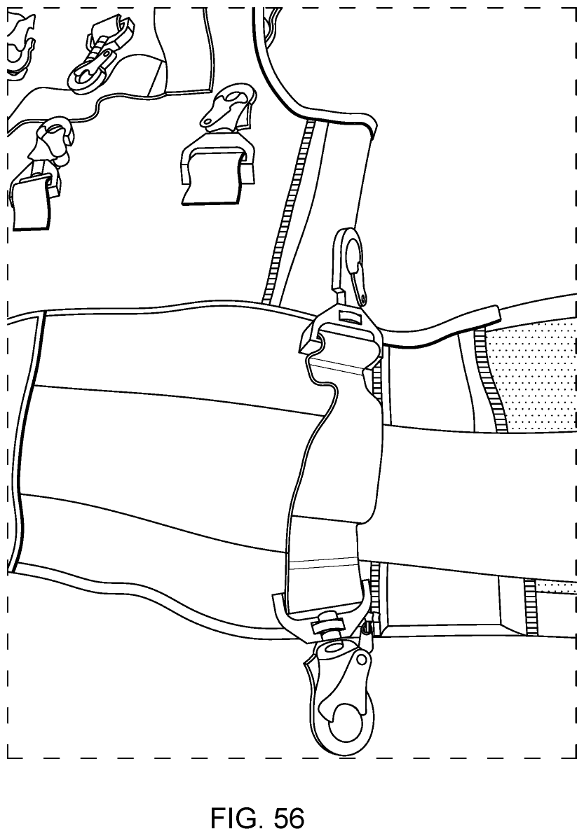

FIG. 56 illustrates a rear, right side view of the compression vest system of FIG. 50, wherein the zipper expansion and retraction mechanism of the vest is in an open position and the zipper expansion and retraction mechanism of the belt is in an open position.

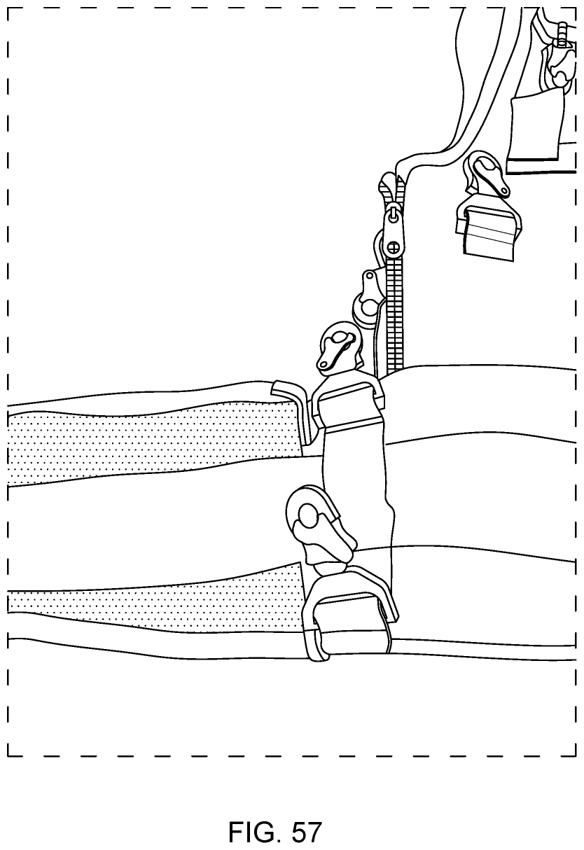

FIG. 57 illustrates a rear, left side view of the compression vest system of FIG. 50, wherein the zipper expansion and retraction mechanism of the vest is in a closed position and the zipper expansion and retraction mechanism of the belt is in a closed position.

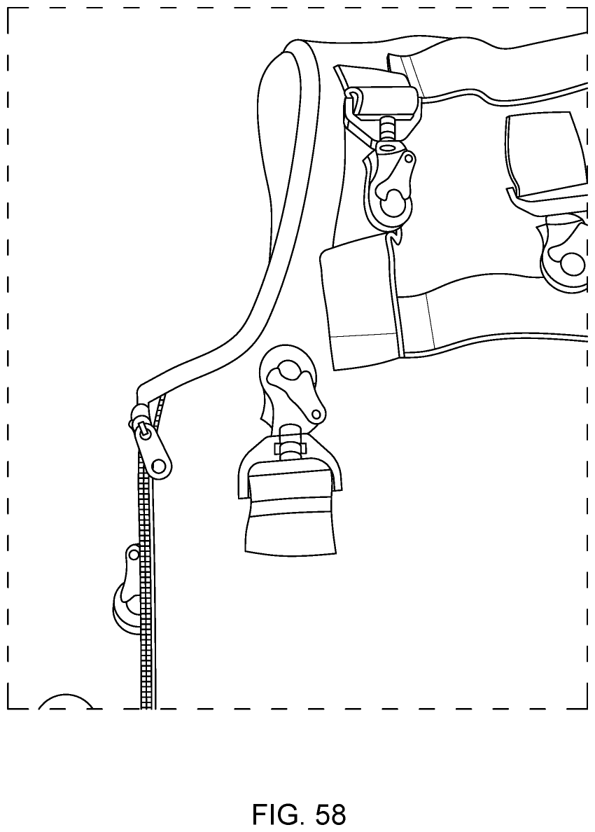

FIG. 58 illustrates a rear, left side view of the vest of the compression vest system of FIG. 50, wherein the zipper expansion and retraction mechanism of the vest is in a closed position.

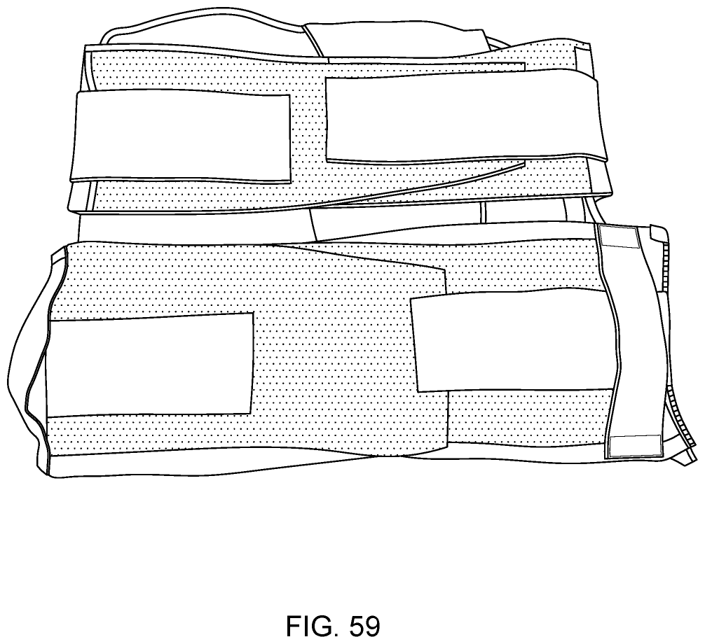

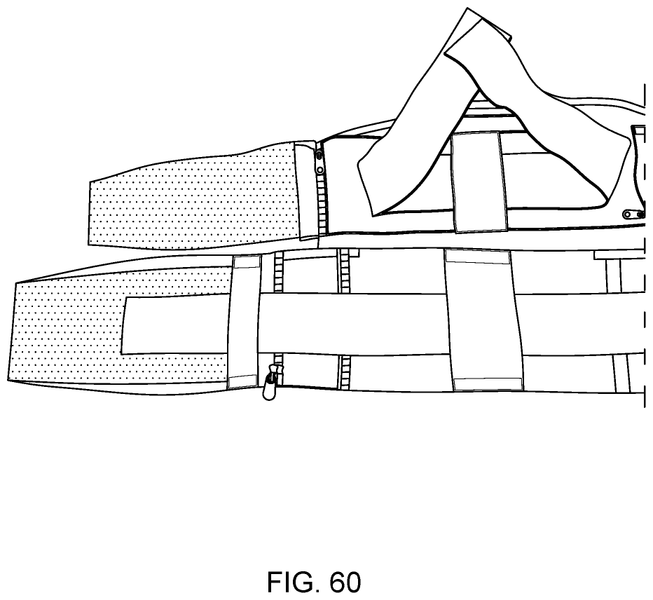

FIG. 59 illustrates a front view of a compression binding vest system according to an aspect of the present disclosure, wherein the compression binding vest system is in a closed position.

FIG. 60 illustrates a rear view of the compression binding vest system of FIG. 59, wherein the zipper expansion and retraction mechanism of the left side of the upper belt is in a closed position and the zipper expansion and retraction mechanism of the left side of the lower belt is in an open position.

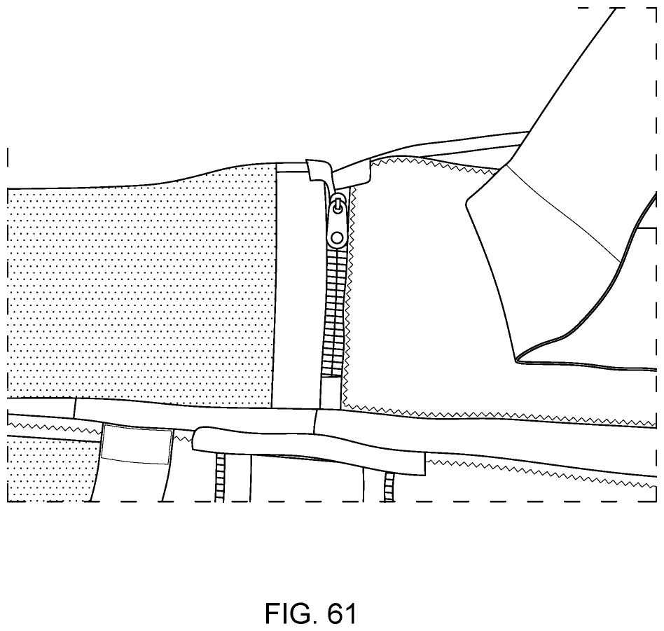

FIG. 61 illustrates a rear view of the compression binding vest system of FIG. 59, wherein the zipper expansion and retraction mechanism of the left side of the upper belt is in the closed position and the zipper expansion and retraction mechanism of the left side of the lower belt is in the open position.

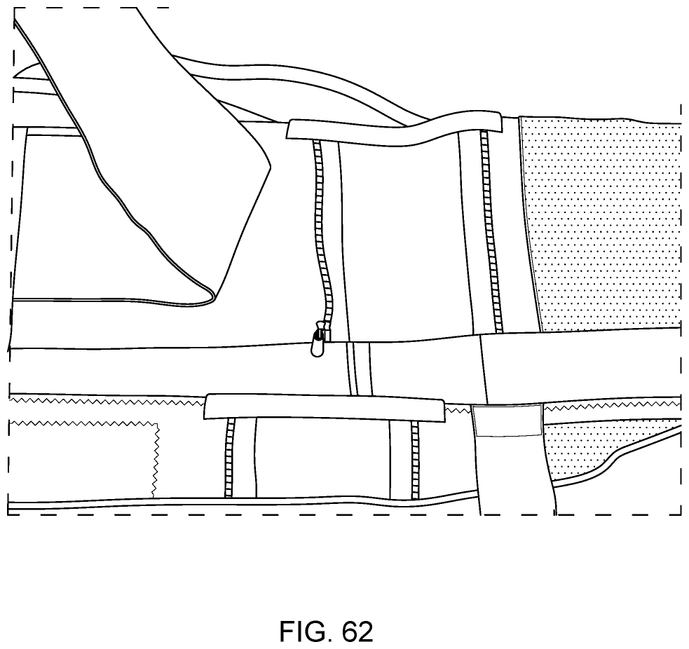

FIG. 62 illustrates a rear view of the compression binding vest system of FIG. 59, wherein the zipper expansion and retraction mechanism of the right side of the upper belt is in the open position and the zipper expansion and retraction mechanism of the right side of the lower belt is in the open position.

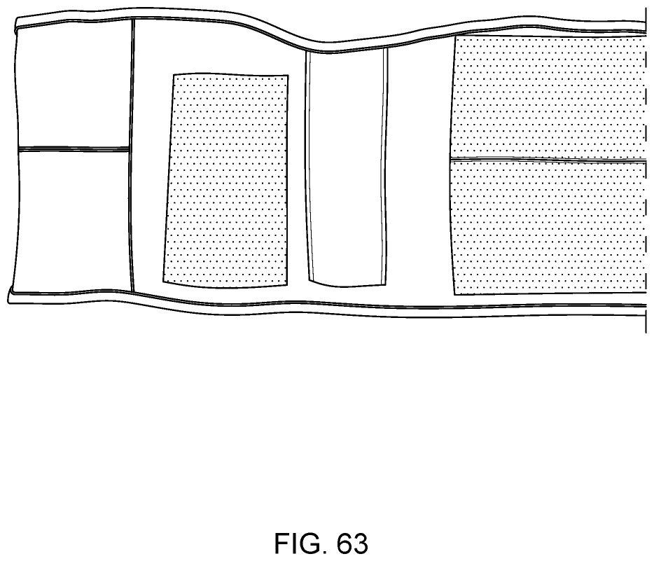

FIG. 63 illustrates a front, right side view of an upper belt of the compression binding vest system of FIG. 59.

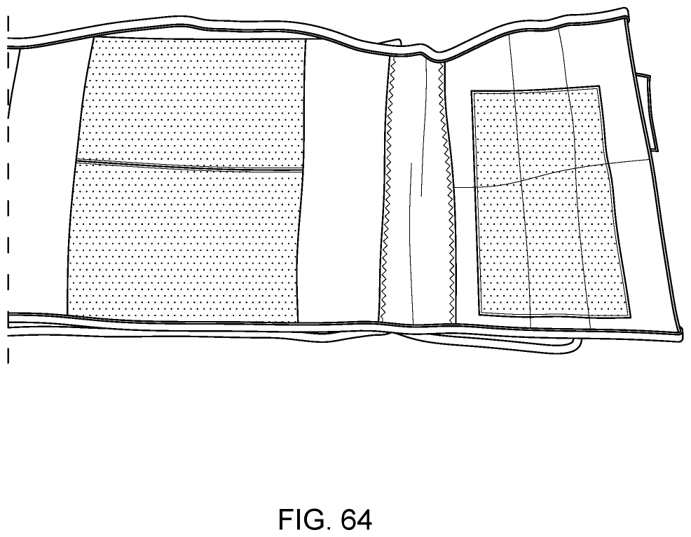

FIG. 64 illustrates a front, left side view of an upper belt of the compression binding vest system of FIG. 59.

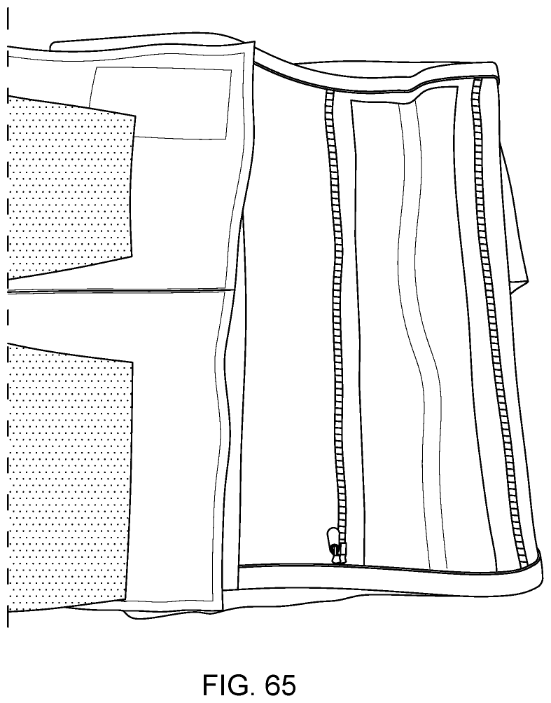

FIG. 65 illustrates a rear, right side view of the compression binding vest system of FIG. 59, wherein the zipper expansion and retraction mechanism of the right side of the upper belt is in the open position.

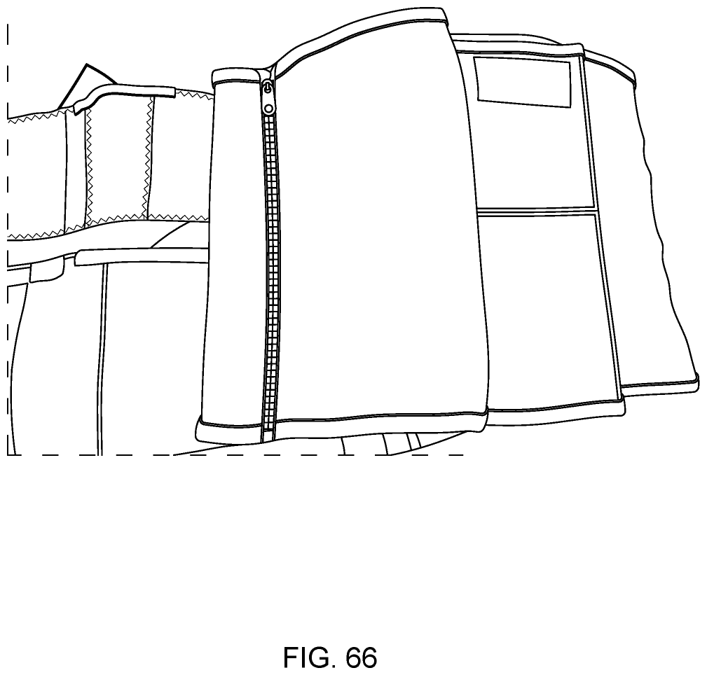

FIG. 66 illustrates a rear, left side view of the compression binding vest system of FIG. 59, wherein the zipper expansion and retraction mechanism of the left side of the upper belt is in the closed position.

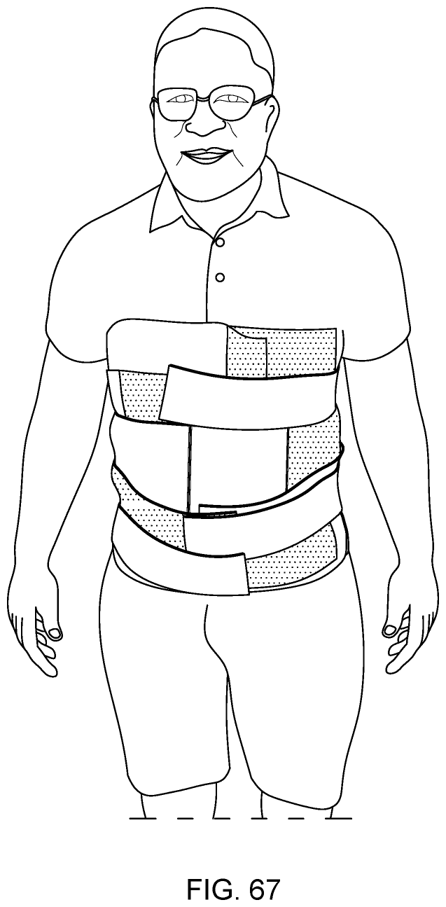

FIG. 67 illustrates a front, environmental view of a compression binding vest system according to an aspect of the present disclosure, wherein the compression binding vest system is in a closed position.

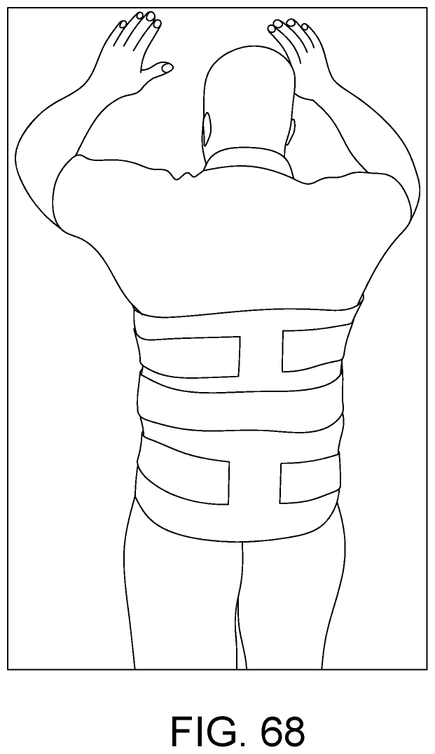

FIG. 68 illustrates a rear, environmental view of the compression binding vest system of FIG. 67, wherein the compression binding vest system is in the closed position.

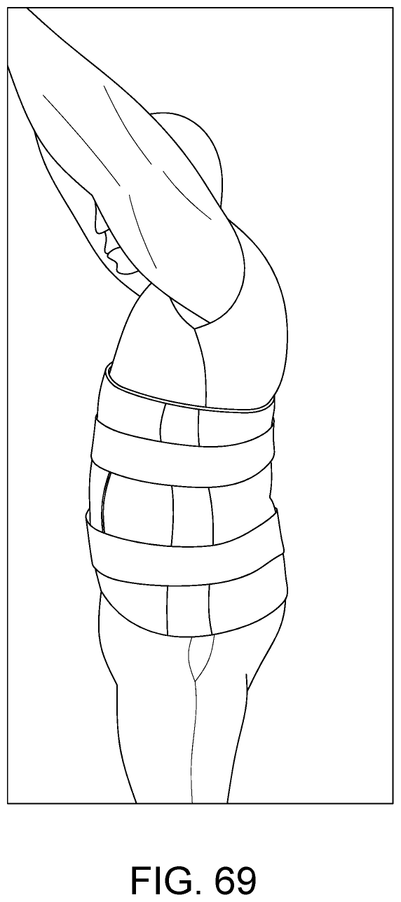

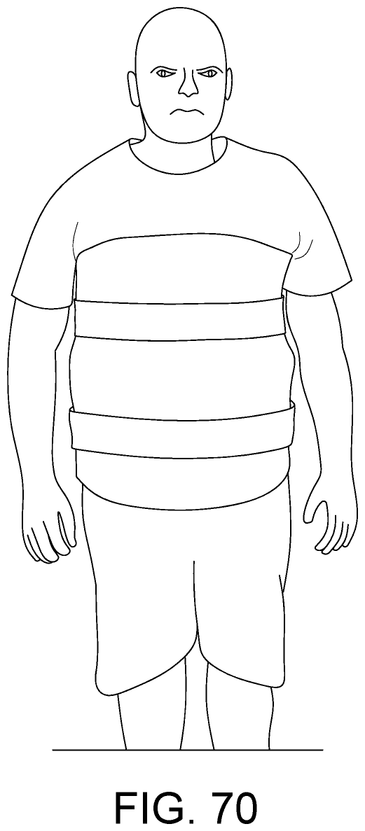

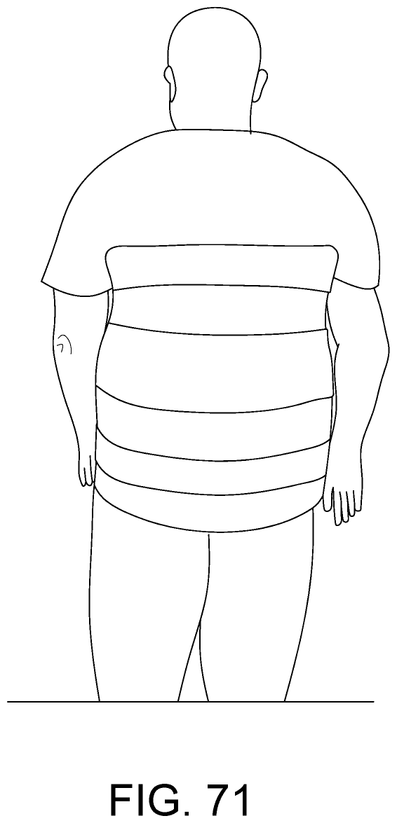

FIG. 69 illustrates a left side view of the compression binding vest system of FIG. 67, wherein the compression binding vest system is in the closed position.

FIG. 70 illustrates a front, environmental view of a compression binding vest system according to an aspect of the present disclosure, wherein the compression binding vest system is in a closed position.

FIG. 71 illustrates a rear, environmental view of the compression binding vest system of FIG. 70, wherein the compression binding vest system is in the closed position.

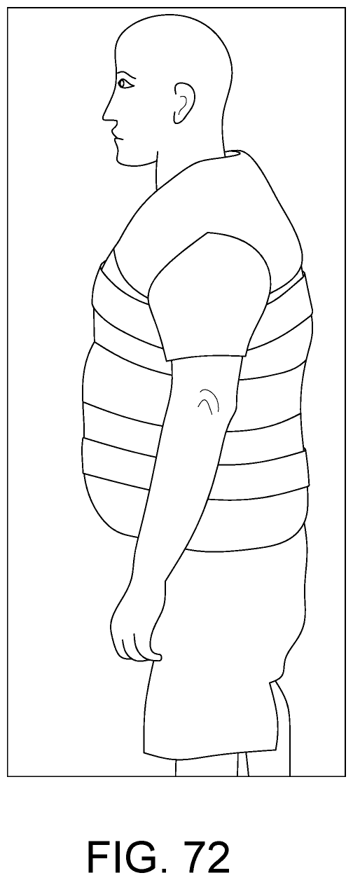

FIG. 72 illustrates a left side view of the compression binding vest system of FIG. 70, wherein the compression binding vest system is in the closed position.

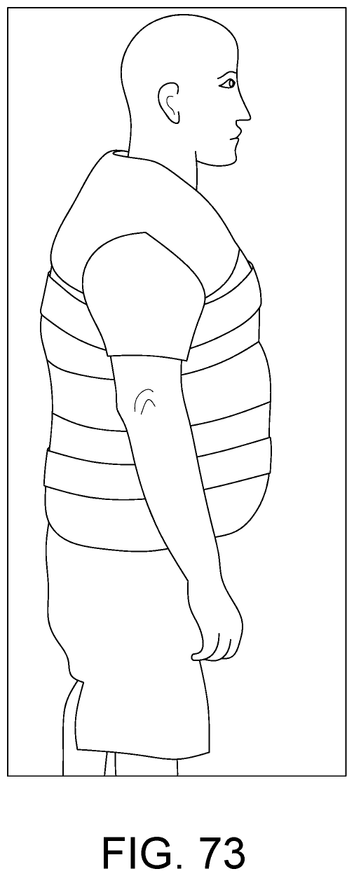

FIG. 73 illustrates a right side view of the compression binding vest system of FIG. 70, wherein the compression binding vest system is in the closed position.

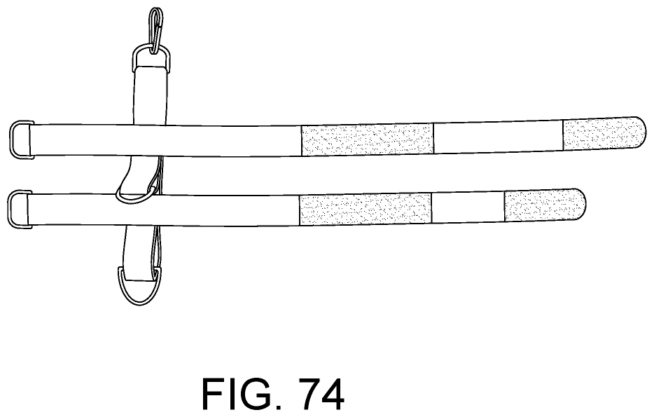

FIG. 74 illustrates a front view of a leg attachment device according to an aspect of the present disclosure.

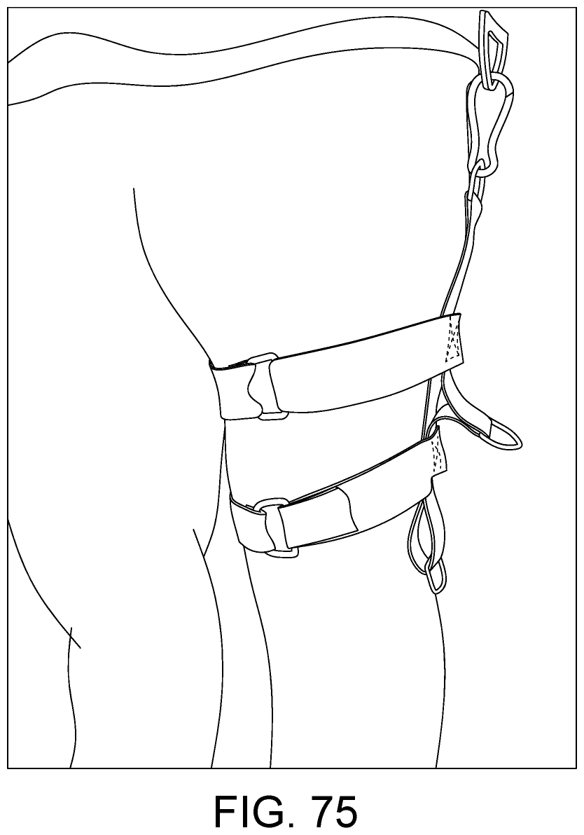

FIG. 75 illustrates a front, left side, perspective view of the leg attachment device of FIG. 74, wherein the leg attachment device is secured to a compression vest system.

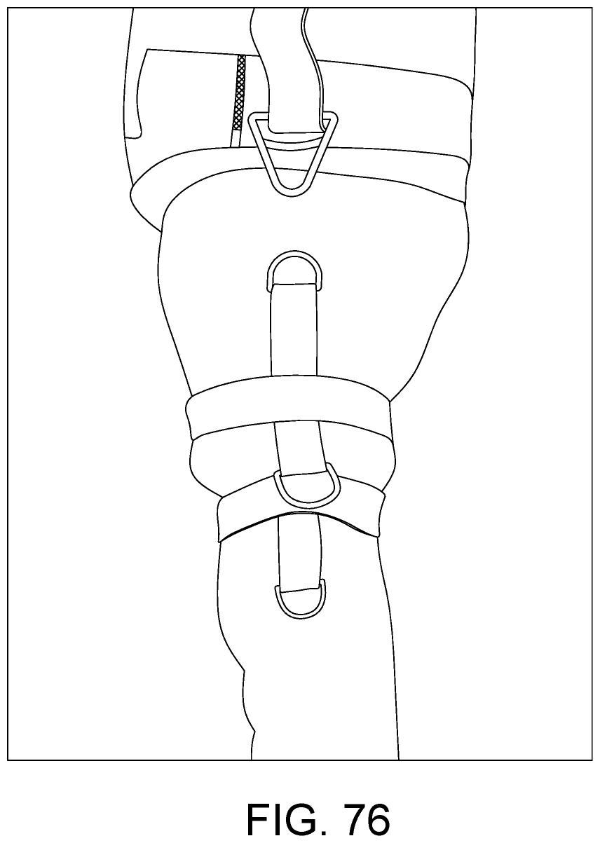

FIG. 76 illustrates a left side view of the leg attachment device of FIG. 74, wherein the leg attachment device is secured to a compression vest system.

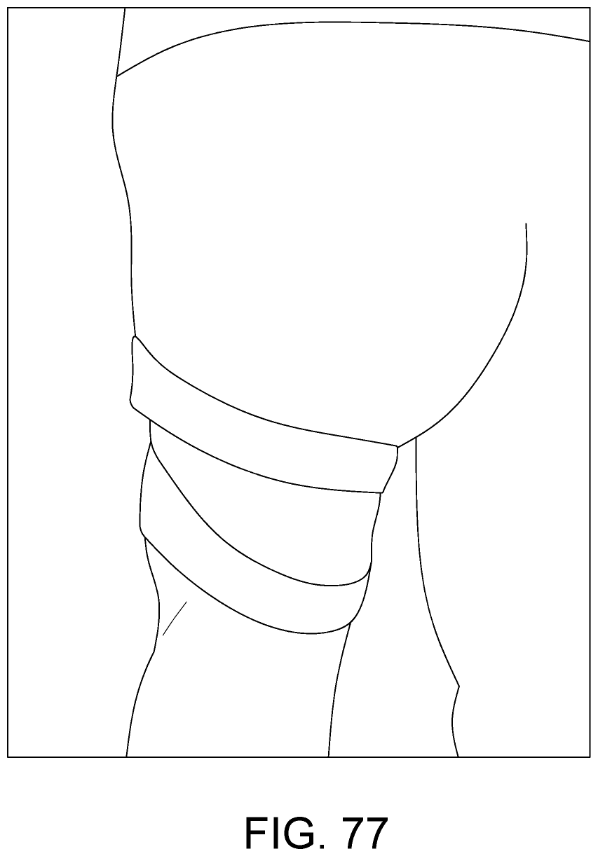

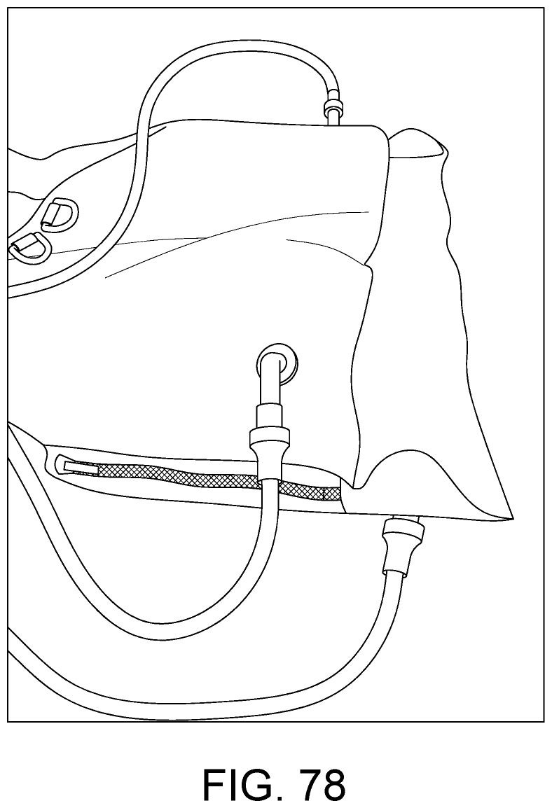

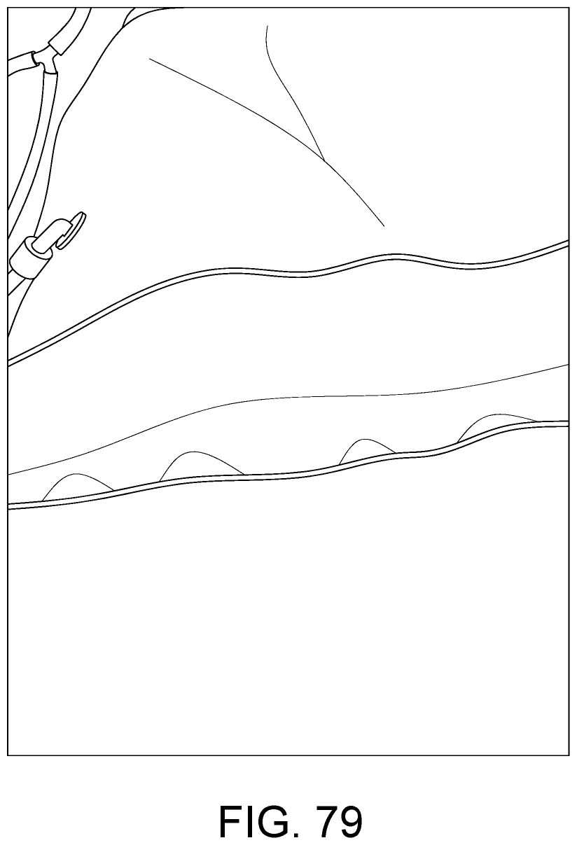

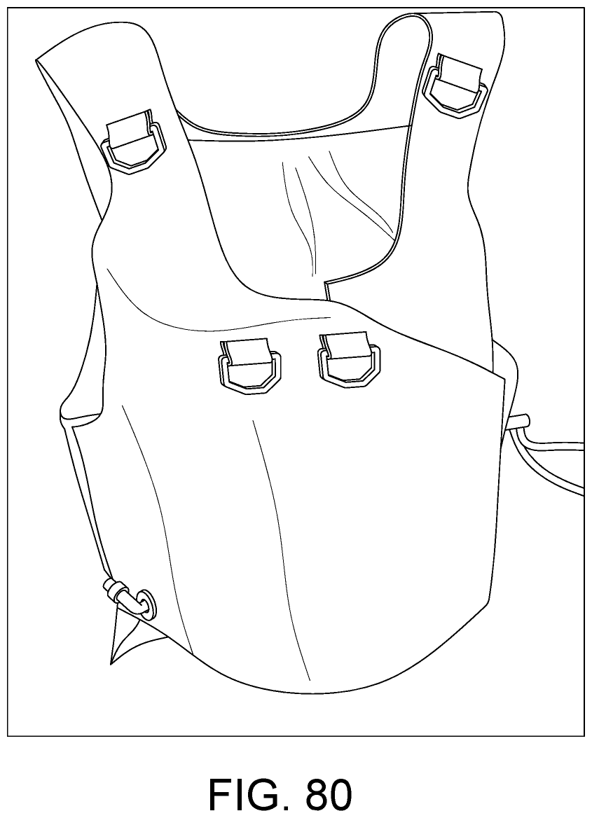

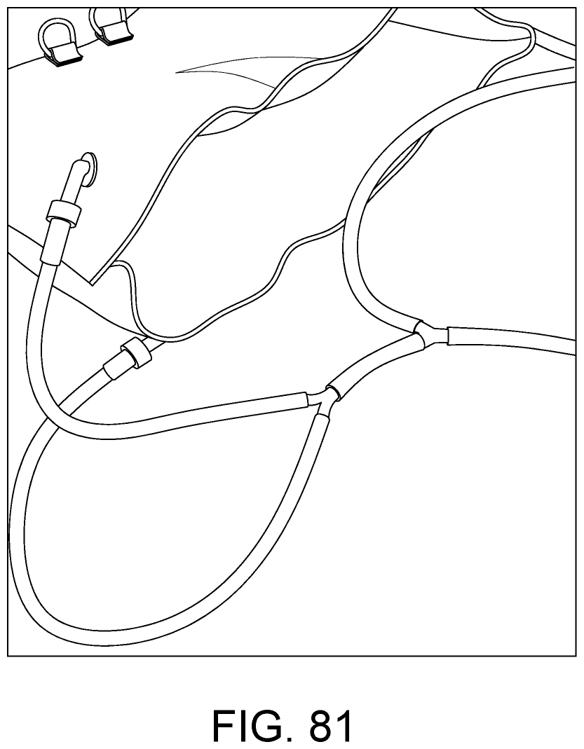

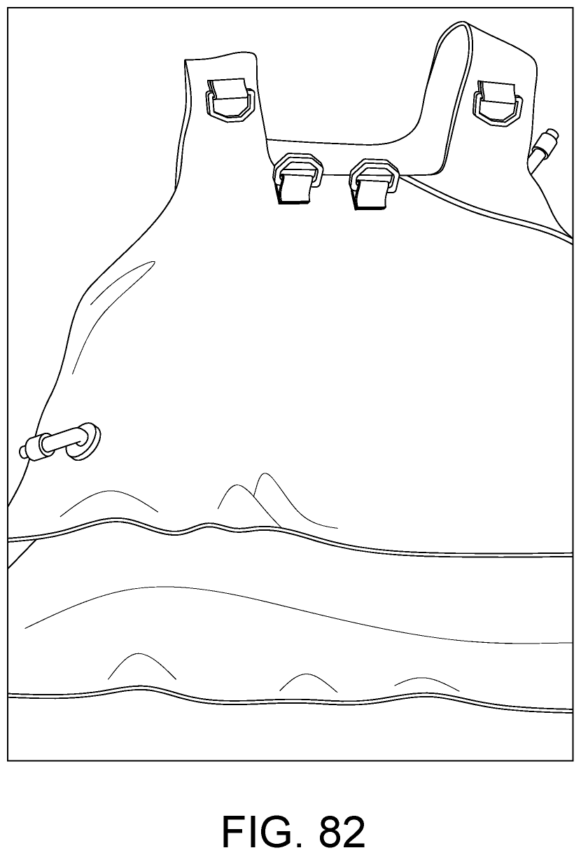

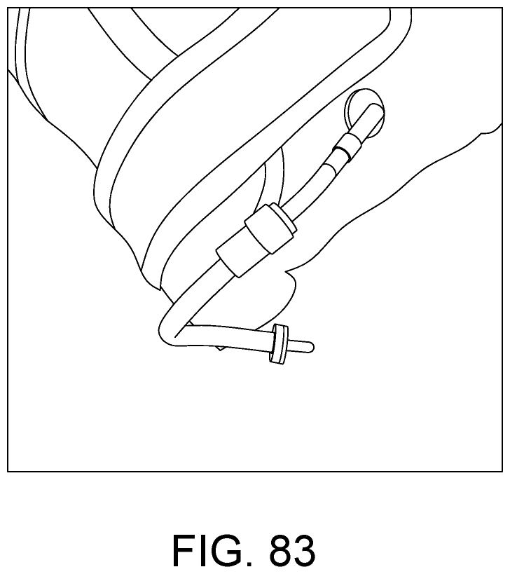

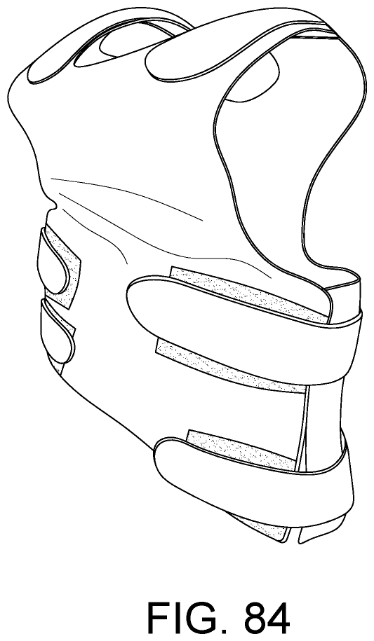

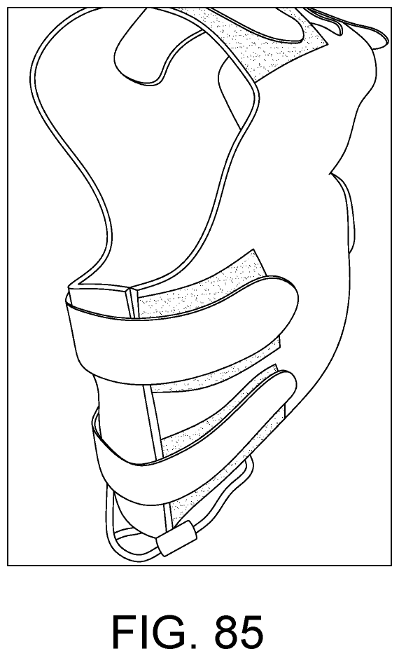

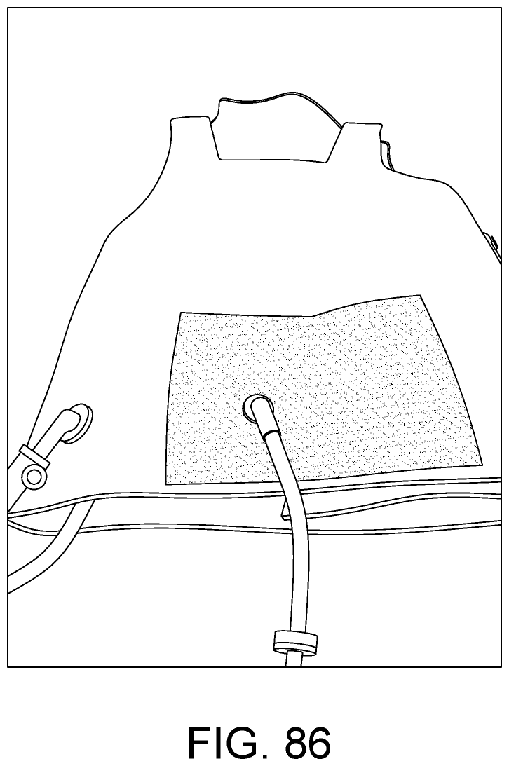

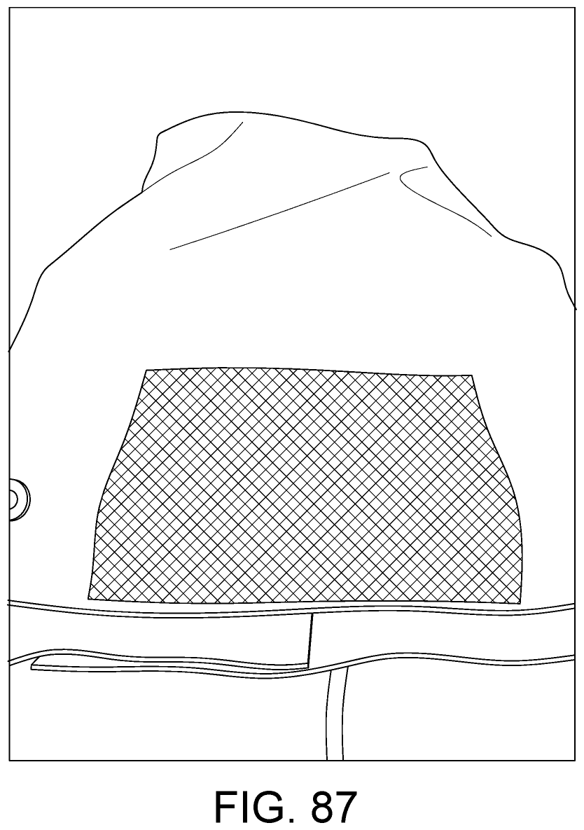

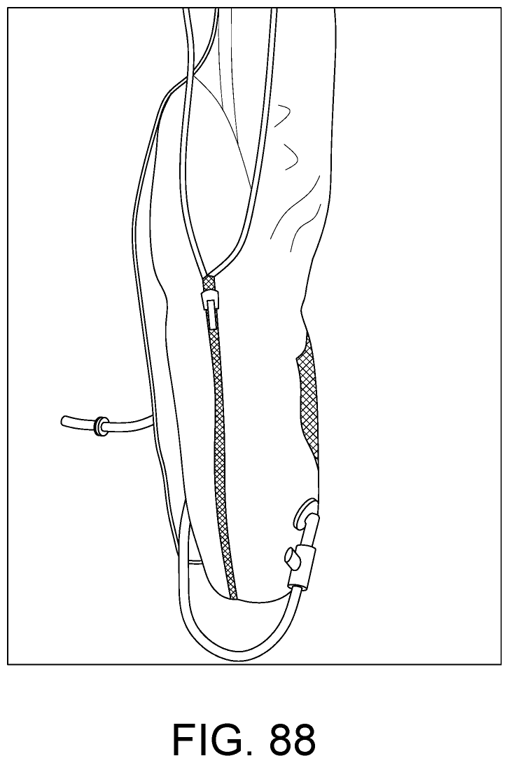

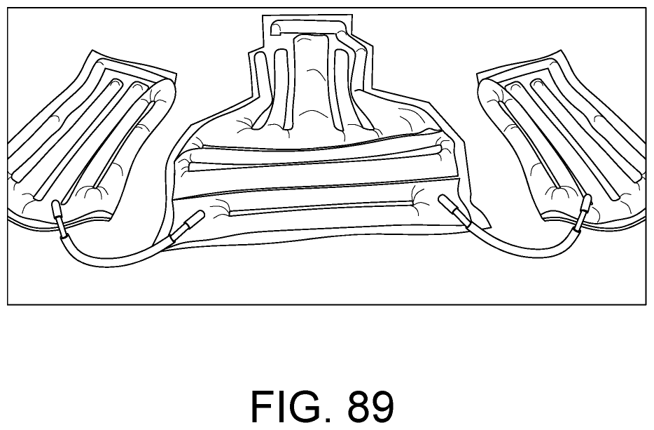

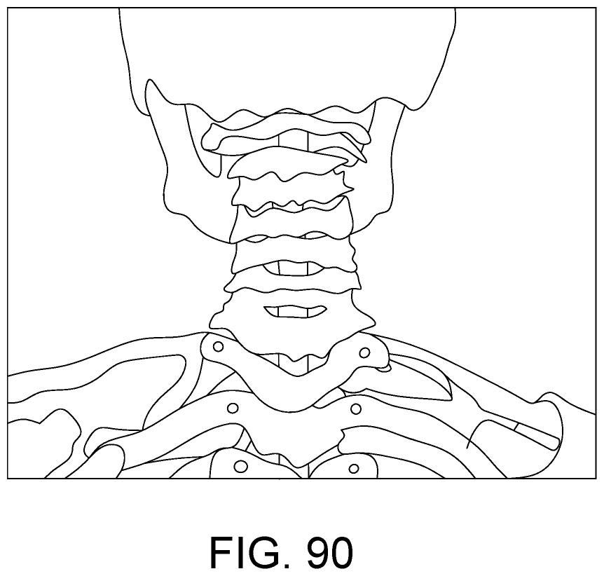

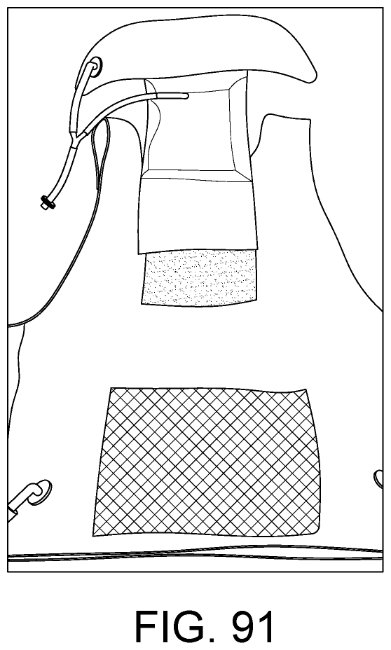

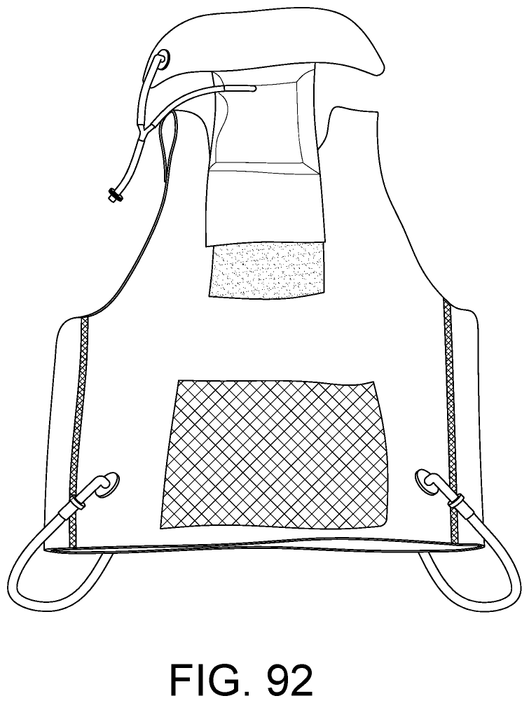

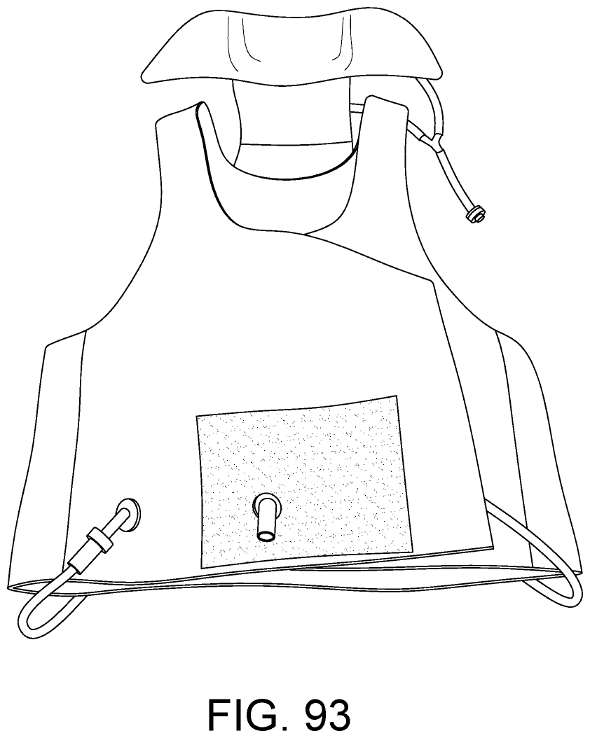

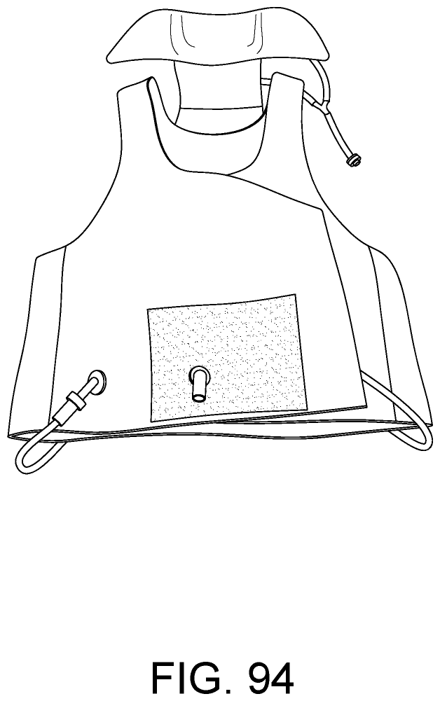

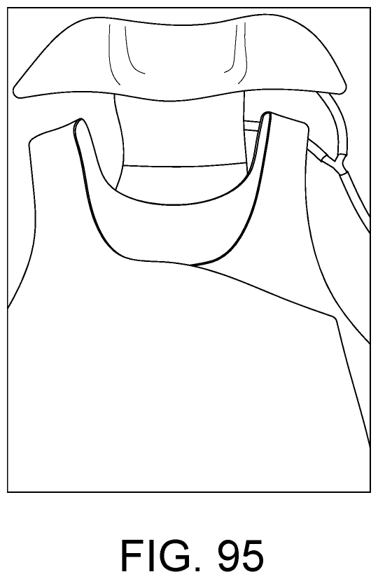

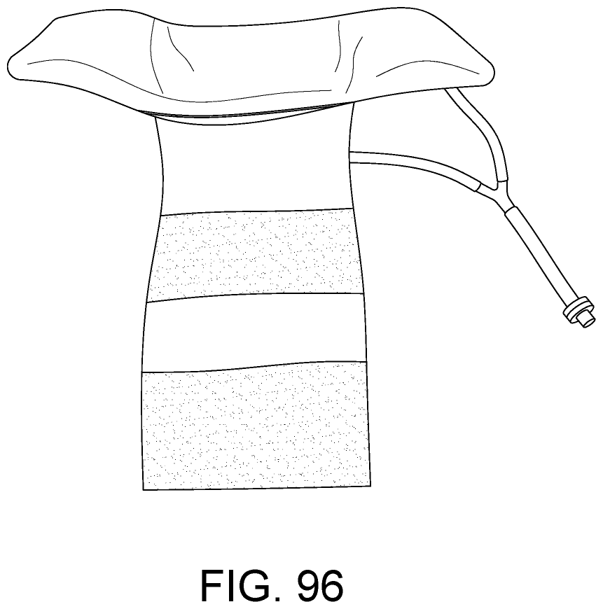

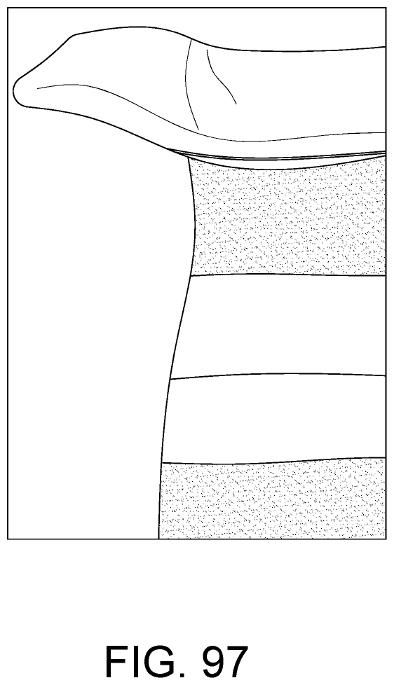

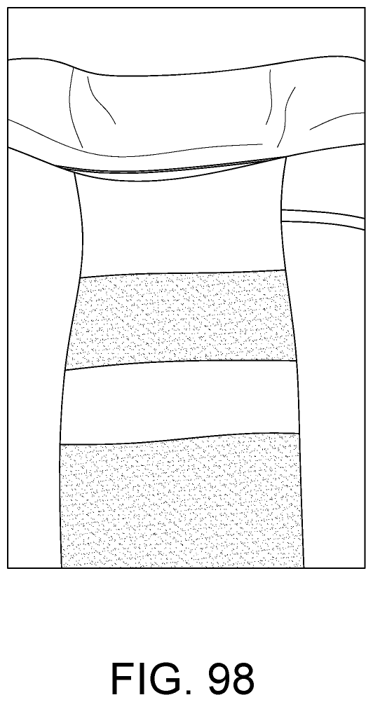

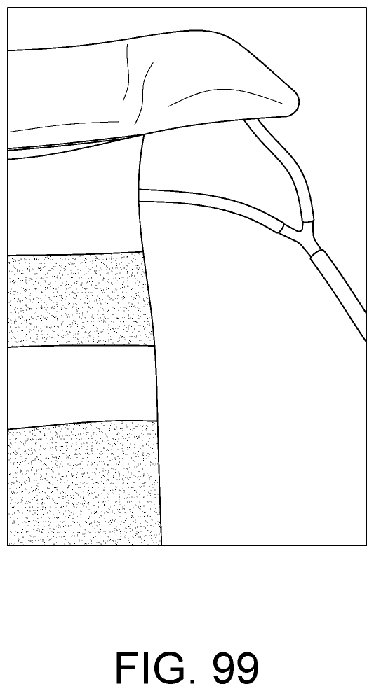

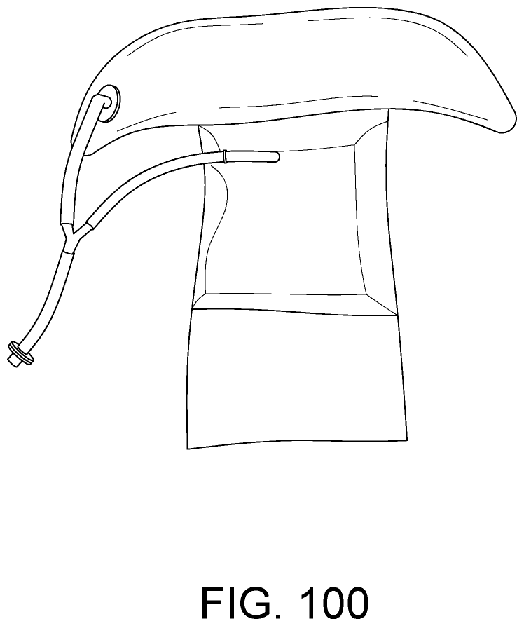

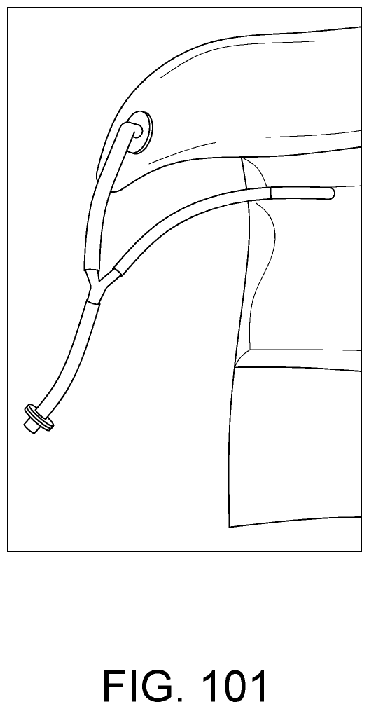

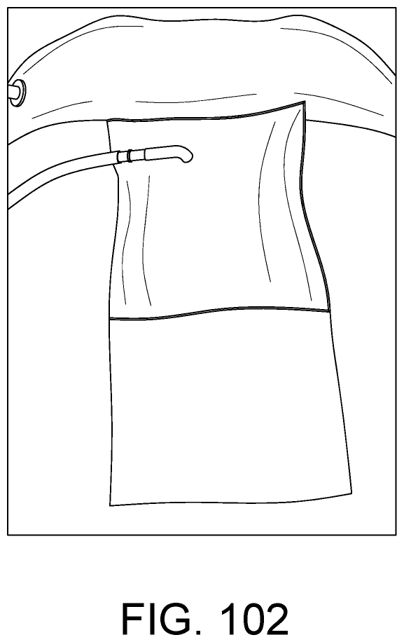

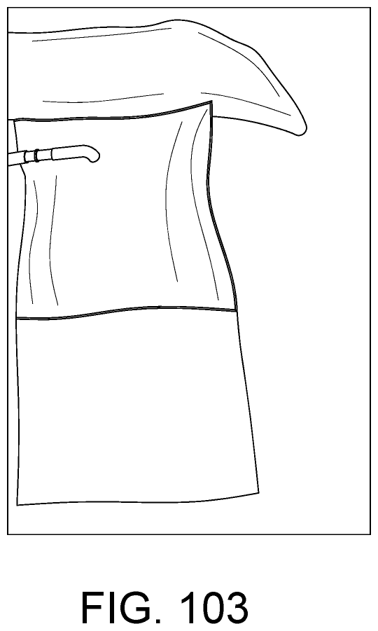

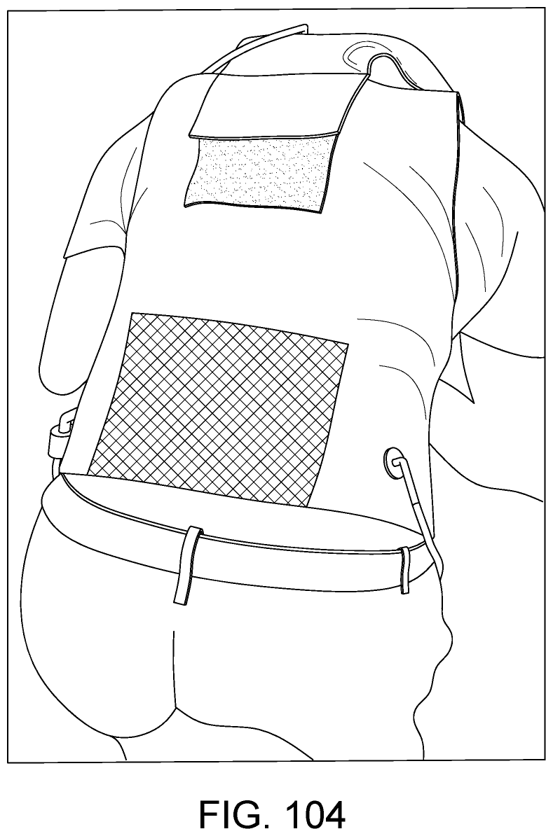

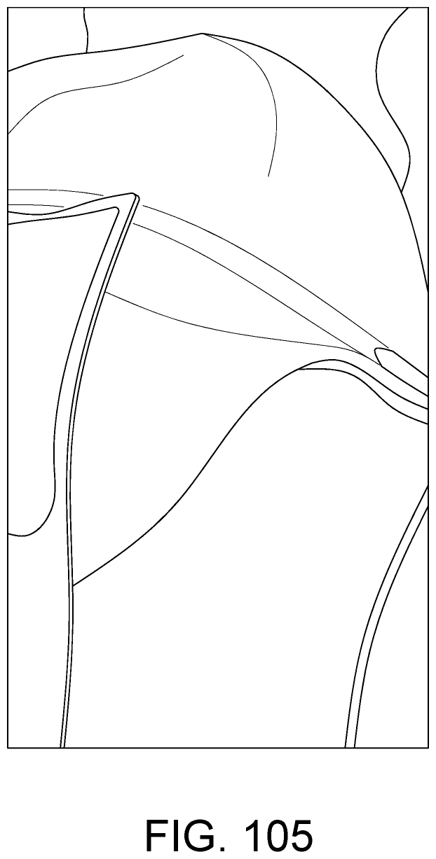

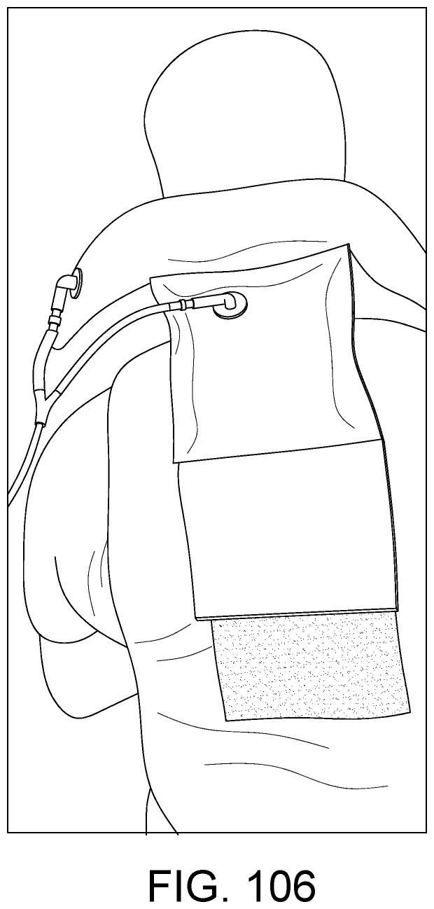

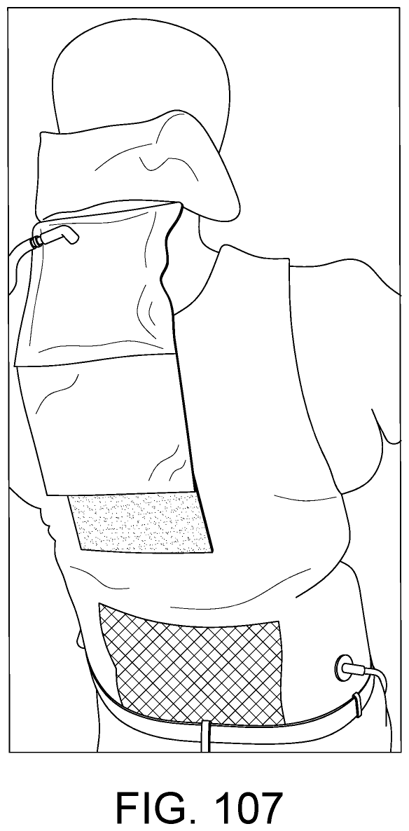

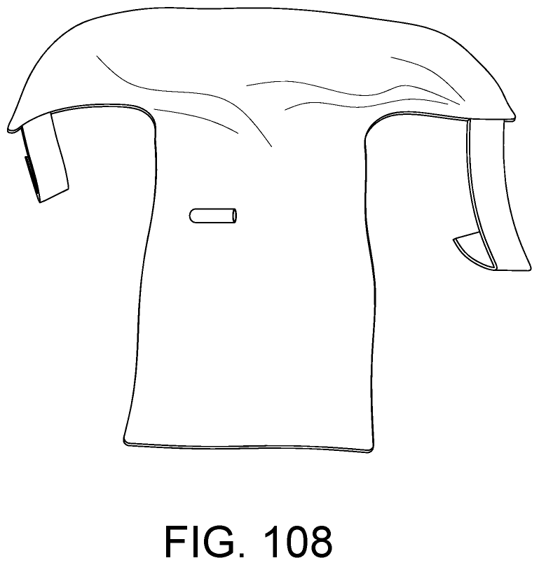

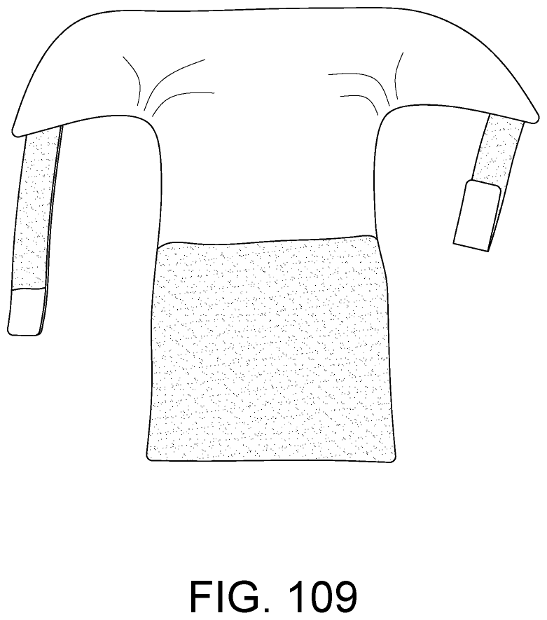

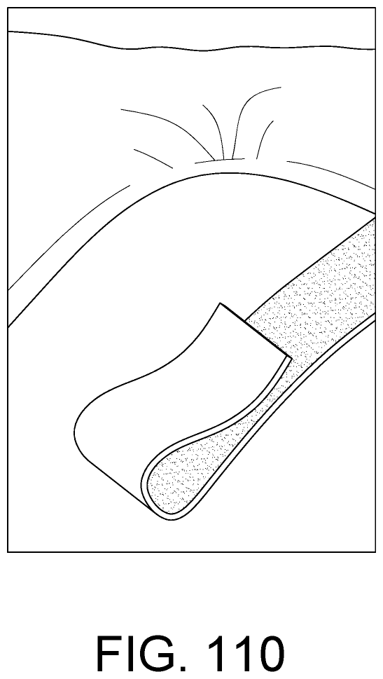

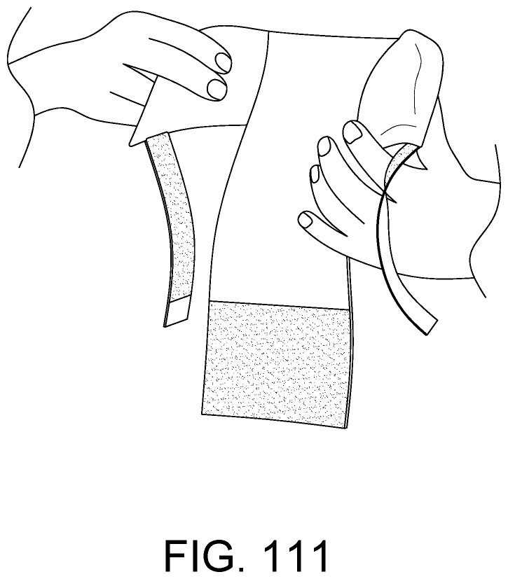

FIG. 77 illustrates a rear, left side, perspective view of the leg attachment device of FIG. 74, wherein the leg attachment device is secured to a compression vest system.

FIG. 42 illustrates a front view of a compression chest protection system according to an aspect of the present disclosure, wherein the compression chest protection system is in a closed position.

FIG. 43 illustrates a rear view of the compression chest protection system of FIG. 42, wherein the compression chest protection system is in a closed position.

FIG. 44 illustrates a rear, right side view of the compression chest protection system of FIG. 42, wherein the compression chest protection system is in an open position.

FIG. 45 illustrates a rear, lower right side view of the compression chest protection system of FIG. 42, wherein the compression chest protection system is in an open position and the zipper expansion and retraction mechanism of the chest protection device is in a closed position.

FIG. 46 illustrates a rear, lower left side view of the compression chest protection system of FIG. 42, wherein the compression chest protection system is in an open position and the zipper expansion and retraction mechanism of the chest protection device is in an open position.

Another aspect of the present disclosure described herein are directed toward a compression chest protection system including a chest protection device and a belt. The compression chest protection system is related to a compression vest system disclosed herein above by including some or several of the features of the compression vest system disclosed herein. According to certain aspects of the present disclosure, the compression vest system includes a vest and a belt that may be worn by a patient and exert forces on the patient that stabilize the vest in a position relative to the patient as grips attached to the vest and belt are used by a caregiver to aid or ensure the safety of a movement of the patient. Further, the belt may include stays that support and stabilize the patient's spine as the belt compresses a torso of the patient. In addition, at least one embodiment or aspect of a compression vest system and/or a compression chest protection system may comprise at least one of the following four additional features, along with some, most or all of the features of the embodiment(s) or aspect(s) of the present invention that do not include these four additional features: (1) at least one zipper expansion and retraction mechanism, such as, but not limited to, on the right side and/or on and left side of the vest and/or on the right side and/or on and left side of the vest, for allowing users of different body shapes and/or sizes to better fit inside the compression vest system during use; (2) Velcro, hook and loop system, and/or any known securing device, mechanism, or system known to one of ordinary skill in the art on the front and rear of the belt and on the belly button area of the vest to increase stabilization, decrease slippage of the belt from the vest, and increase security of the belt to the vest (especially during transfer or use); (3) at least one swivel hook, ring, attachment ring, and/or any known securing device, mechanism, or system known to one of ordinary skill in the art that are dependent on the type(s) of an individual's needs, sports, therapy, rehabilitation, and settings (as a non-limiting example, this third additional feature is positioned on the vest and belt (five: two on hip of each side of the belt and one on the back side of the belt) to the attachment point); and (4) a leg attachment device for detachably securing a compression vest system to a leg of a user for better stabilization of the compression vest system to the user during transfer or use. Preferably, an embodiment or aspect of a compression vest system comprises most or all of these four additional features.

With regard to the leg attachment device, the leg attachment device may be secured above the knee or, alternatively, below the knee of the user or patient. The above knee attachment may be called Above Knee Strap (AKS) while the below knee attachment may be called Below Knee Strap (BKS). As a non-limiting example, an AKS or, alternatively, BKS for both legs may have multiple attachment points (such as, but not limited to, at least three) for each strap. Alternatively, various pieces of attachment devices for the straps may be used with regard to size, shape, strength, weight, and material.

The compression chest protection system of the present disclosure is an improvement over baseball chest protectors by including one or more of the following features:

(1) geometrical shape deflecting members, such as, but not limited to, diamonds, rectangles, pentagons, and squares, are positioned on the front of the chest protection device;
(2) back of the chest protection device have straps, quick release snaps, tum mechanisms (loosening or tightening strap(s)), and/or other securing and/or releasing members described above;
(3) the compression belt limits the chest protection device from sliding up and down, turning, rotating, and/or puffing outwardly;
(4) more body conforming design; and
(5) the compression belt may include hooks, straps, quick release snaps, tum mechanisms (loosening or tightening strap(s)), and/or other securing and/or releasing members described above.

Bladders increase the compression forces to help stabilize the spin al region, the thoracic region, and nearby regions. Also, helps to protect any areas that are loose or have extra spacing within the vest by the compartmentalized bladders (multiple chambers can be vertical, horizontal, and/or any other configurations). At least one chamber but preferably multiple chambers in the device.

Also use bladders with other materials, such as, but not limited to, foam; lexane plates; metal, any rigid plastics, any material known in the art, and any combination thereof.

It will be appreciated that the foregoing description provides examples of the disclosed compression vest system and techniques for applying the compression vest system. These examples given above are merely illustrative and are not meant to be an exhaustive list of all possible designs, aspects, applications or modifications of the disclosure. Further, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated.

I claim:

1. A vest comprising:
a first panel including a first flap;
a second panel including a second flap;
a back panel extending perpendicular to a longitudinal axis of the vest from the first panel to the second panel; and
a plurality of grips attached to the first panel, the second panel, and the back panel,
wherein the back panel defines a first arm access with the first panel and a second arm access with the second panel,
wherein portions of the first panel, second panel, and back panel disposed along a longitudinal axis of the vest below the first arm access and the second arm access define a body of the vest,
wherein the first flap is configured to overlap the second flap such that the first flap and the second flap define a closure of the vest and the first panel and the second panel define a collar of the vest,
wherein the first flap includes a first front attachment segment and the second flap includes a second front attachment segment configured to attach to the first front attachment segment in a closed state of the vest, and
wherein an area of an attachment configuration between the first front attachment segment and the second front attachment segment in the closed state of the vest is equal to at least ⅔ of an area having a length equal to a length of the body and a width equal to ¼ of a maximum width of the body.

2. The vest of claim 1,
wherein a longitudinal axis of the closure is offset from the longitudinal axis of the vest.

3. The vest of claim 1,
wherein the plurality of grips includes a first front grip and a second front grip,
wherein the first front grip is positioned between the first arm access and a portion of an inner edge of the first panel that defines a collar in the closed stated of the vest, and
wherein the second front grip is positioned between the second arm access and a portion of an inner edge of the second panel that defines the collar in the closed stated of the vest.

4. The vest of claim 1,
wherein the first front attachment segment extends:
continuously along the longitudinal axis,
from a lower end of the body to at least a location corresponding to a lower end of the first arm access, and
adjacent to an inner edge of the first panel, and
wherein the second front attachment segment extends:
continuously along the longitudinal axis,
from a lower end of the body to at least a location corresponding to a lower end of the second arm access, and
adjacent to an inner edge of the second panel.

5. The vest of claim 1,
wherein each of the first front attachment segment and the second attachment segment is defined by a layer of interlocking material that is fastened to a respective one of the first flap and the second flap by stitching, and
wherein each of the plurality of grips includes a pair of grip attachments, each of the grip attachments including an end of a respective grip attached to the vest by reinforcement stitching.

6. The vest of claim 5,
wherein the plurality of grips includes a front grip positioned between the second arm access and a portion of an inner edge of the second panel that defines the collar in the closed stated of the vest, and
wherein one of a respective pair of grip attachments of the front grip is covered by a portion of the second front attachment segment that is attached to the vest by stitching.

7. The vest of claim 1,
wherein the first front attachment segment includes a plurality of first front attachment segments that are spaced apart along the longitudinal axis, and
wherein the second front attachment segment includes a plurality of second front attachment segments that are spaced apart along the longitudinal axis.

8. The vest of claim 1, further comprising:
a back attachment segment positioned on an exterior surface of the back panel adjacent to a lower end of the body,
wherein the back attachment segment extends perpendicular to the longitudinal axis over a distance equal to at least half of the maximum width of the body.

9. The vest of claim 8, further comprising:
a back attachment segment positioned on an interior surface of the back panel adjacent to a lower end of the body,
wherein the back attachment segment extends perpendicular to the longitudinal axis over a distance equal to the maximum width of the body.

10. A compression vest system, the compression vest system comprising:
a vest including:
a back panel including a back attachment segment provided on an exterior surface of the back panel,
a first panel extending from the back panel,
a second panel extending from the back panel, and
a plurality of grips attached to at least the first panel and the second panel; and
a belt including:
a pair of flanks,
a first belt attachment segment positioned on an interior surface of an end of one of the pair of flanks,
a second belt attachment segment positioned on interior surface of the belt between the pair of flanks and configured to attach to the back attachment segment, and
a pair of elastic tension bands attached to an exterior surface of the belt between the pair of flanks,
wherein the first panel and the second panel define a closure of the vest that extends along a longitudinal axis of the vest and has a width equal to at least 4 of a maximum width of the vest,
wherein the closure is configured to exert first compressive forces on an individual wearing the vest,
wherein the pair of flanks are configured to wrap around the vest such that one flank overlaps an other flank and the pair of flanks exert second compressive forces on the individual, and
wherein each of the pair of elastic tension bands is configured to be attached to a third belt attachment segment positioned on an exterior surface of a respective one of the pair of flank in a state of tension and increase the second compressive forces.

11. The compression vest system of claim 10,
wherein the back panel defines a first arm access with the first panel and a second arm access with the second panel,
wherein portions of the first panel, second panel, and back panel disposed along the longitudinal axis of the vest below the first arm access and the second arm access define a body of the vest,
wherein the closure includes a first front attachment segment of the first panel and a second front attachment segment of the second panel, and
wherein an area of an attachment configuration between the first front attachment segment and the second front attachment segment in a closed state of the vest is equal to at least ⅔ of an area having a length equal to a length of the body and a width equal to ¼ of a maximum width of the body.

12. The compression vest system of claim 11,
wherein the first front attachment segment extends:
continuously along the longitudinal axis,
from a lower end of the body to at least a location corresponding to a lower end of the first arm access, and
adjacent to an inner edge of the first panel, and
wherein the second front attachment segment extends:
continuously along the longitudinal axis,
from a lower end of the body to at least a location corresponding to a lower end of the second arm access, and
adjacent to an inner edge of the second panel.

13. The compression vest system of claim 11,
wherein the first front attachment segment includes a plurality of first front attachment segments that are spaced apart along the longitudinal axis, and
wherein the second front attachment segment includes a plurality of second front attachment segments that are spaced apart along the longitudinal axis.

14. The compression vest system of claim 10,
wherein the belt includes a pair of belt grips attached to the exterior surface of the belt between the pair of flanks.

15. The compression vest system of claim 10,
wherein the plurality of grips includes a body grip attached to each side of the body.

16. The compression vest system of claim 15,
wherein each body grip is positioned on the body along the longitudinal axis above the back attachment segment.

17. The compression vest system of claim 11, further comprising:
a cloak having a first surface and a second surface and including:
a first cloak attachment segment positioned on the first surface,
a second cloak attachment segment position on the second surface, and
a plurality of grips positioned on the first surface,
wherein the back attachment segment of the vest is a first back attachment segment of a plurality of the back attachment segments that includes a second back attachment segment positioned on the exterior surface of the back panel and a third back attachment segment positioned on an interior surface of the back panel,
wherein the first cloak attachment segment is configured to attach to at least one of the plurality of the back attachment segments and the second cloak attachment segment is configured to attach to the second belt attachment segment, and
wherein the cloak is configured to attach to the vest and the belt and extend past legs of an individual wearing the vest.

18. A method of transferring a patient, the method comprising:
providing a vest including a first panel, a second panel, and a back panel extending from the first panel and the second panel;
positioning the vest on the patient such that the vest exerts first compressive forces on the patient;
wrapping a pair of flanks of the belt around the vest such that the belt exerts second compressive forces on the patient;
attaching an end of each of a pair of elastic tension bands to an attachment segments positioned on respective one of the pair of the flanks such that each elastic tension band is attached in a state of tension and increases a magnitude of the second compressive forces; and
grasping one or more of a plurality of grips attached to the vest and performing at least one of guiding and supporting a movement of the patient.

19. The method of claim 18,
wherein the back panel defines a first arm access with the first panel and a second arm access with the second panel,
wherein portions of the first panel, second panel, and back panel disposed along a longitudinal axis of the vest below the first arm access and the second arm access define a body of the vest, and
wherein the positioning includes attaching a first front attachment segment of the first panel to a second front attachment segment of the second panel such that an area of a configuration of portions of the first front attachment segment configured to mirror and attach to portions of the second front attachment segment in a closed state of the vest is equal to at least ⅔ of an area having a length equal to a length of the body and a width equal to ¼ of a maximum width of the body.

20. The method of claim 18, further comprising:
attaching a cloak to the vest and attaching the belt to the cloak before the wrapping of the pair of flanks around the patient;
positioning the cloak beneath a body of the patient;
grasping a plurality of grips attached to the cloak during the grasping of the one or more of the plurality of grips attached to the vest;
applying a force to each grip being grasped to lift the patient; and
carrying the patient while continuing to grasp each grip.
